(19) United States
(12) Patent Application Publication
(10) Pub. No.: US 2022/0400986 A1
(43) Pub. Date: Dec. 22, 2022

(54) SYSTEMS, DEVICES, AND METHODS FOR PHYSIOLOGICAL PARAMETER ANALYSIS AND RELATED GRAPHICAL USER INTERFACES

(71) Applicant: ABBOTT DIABETES CARE INC., Alameda, CA (US)

(72) Inventors: Deepak Khanal, Lake Forest, IL (US); Timothy C. Dunn, San Francisco, CA (US); Luca Birolini, Southlake, TX (US); John M. Schullian, Cary, IL (US); Gary Alan Hayter, Oakland, CA (US)

(73) Assignee: ABBOTT DIABETES CARE INC., Alameda, CA (US)

(21) Appl. No.: 17/832,537

(22) Filed: Jun. 3, 2022

Related U.S. Application Data
(60) Provisional application No. 63/328,078, filed on Apr. 6, 2022.
(60) Provisional application No. 63/279,509, filed on Nov. 15, 2021.
(60) Provisional application No. 63/279,015, filed on Nov. 12, 2021.
(60) Provisional application No. 63/196,677, filed on Jun. 3, 2021.

(51) Int. Cl. A61B 5/145; G16H 10/60; A61B 5/00

(52) U.S. Cl. CPC A61B 5/14532; G16H 10/60; A61B 5/7405; A61B 5/743; A61B 5/746; A61B 5/0004; A61B 5/0022; A61B 2562/08

(57) ABSTRACT

A method can include receiving, using one or more processors, a first record including a first data associated with a personal identification from a first database, receiving, using the one or more processors, a second record including a second data associated with a user identification from a second database, pairing, using the one or more processors, the first data and the second data based upon a shared data item contained in the first record and the second record, and displaying, using one or more processors, a report based upon the first data and the second data.

### CROSS REFERENCE TO RELATED APPLICATIONS

This application claims the benefit of U.S. Provisional Patent Application No. 63/196,677 filed Jun. 3, 2021, U.S. Provisional Patent Application No. 63/279,015 filed Nov. 12, 2021, U.S. Provisional Patent Application No. 63/279,509 filed Nov. 15, 2021, U.S. Provisional Patent Application No. 63/328,078 filed Apr. 6, 2022, which are hereby incorporated by reference in their entireties.

### FIELD

The subject matter described herein relates generally to improved analyte monitoring systems, as well as methods and devices relating thereto.

### BACKGROUND

The detection and/or monitoring of analyte levels, such as glucose, ketones, lactate, oxygen, hemoglobin A1C, albumin, alcohol, alkaline phosphatase, alanine transaminase, aspartate aminotransferase, bilirubin, blood urea nitrogen, calcium, carbon dioxide, chloride, creatinine, hematocrit, lactate, magnesium, oxygen, pH, phosphorus, potassium, sodium, total protein, uric acid, etc., or the like, can be important to the health of an individual having diabetes. Patients suffering from diabetes mellitus can experience complications including loss of consciousness, cardiovascular disease, retinopathy, neuropathy, and nephropathy. Diabetics are generally required to monitor their glucose levels to ensure that they are being maintained within a clinically safe range, and may also use this information to determine if and/or when insulin is needed to reduce glucose levels in their bodies, or when additional glucose is needed to raise the level of glucose in their bodies.

Growing clinical data demonstrates a strong correlation between the frequency of glucose monitoring and glycemic control. Despite such correlation, however, many individuals diagnosed with a diabetic condition do not monitor their glucose levels as frequently as they should due to a combination of factors including convenience, testing discretion, pain associated with glucose testing, and cost.

To increase patient adherence to a plan of frequent glucose monitoring, in vivo analyte monitoring systems can be utilized, in which a sensor control device may be worn on the body of an individual who requires analyte monitoring. To increase comfort and convenience for the individual, the sensor control device may have a small form-factor and can be applied by the individual with a sensor applicator. The application process includes inserting at least a portion of a sensor that senses a user's analyte level in a bodily fluid located in a layer of the human body, using an applicator or insertion mechanism, such that the sensor comes into contact with a bodily fluid. The sensor control device may also be configured to transmit analyte data to another device, from which the individual, her health care provider (“HCP”), or a caregiver can review the data and make therapy decisions.

Despite their advantages, however, some people are reluctant to use analyte monitoring systems for various reasons, including the complexity and volume of data presented, a learning curve associated with the software and user interfaces for analyte monitoring systems, and an overall paucity of actionable information presented.

Thus, needs exist for improved digital and graphical user interfaces for analyte monitoring systems, as well as methods and devices relating thereto, that are robust, user-friendly, and provide for timely and actionable responses.

Additionally, certain patient information, particularly as it relates to laboratory test results, currently resides at various healthcare care organization's (HCO) local computer networks (e.g., electronic medical/health records). Such information is recorded and stored on EMR systems using patient identification that is unique to each HCO. Similarly, analyte monitoring systems often store analyte measurements on a centralized database using user identification (e.g., username, email address, etc.).

Thus, needs exist for systems and methods for bi-directional communication so that patient data from HCOs can be paired with data from analyte measurement systems.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

The achieve these and other advantages and in accordance with the purpose of the disclosed subject matter, as embodied and broadly described, the disclosed subject matter is directed to systems monitoring glucose. According to an embodiment, a system for monitoring glucose can include a sensor control device and a reader device. The sensor control device can include an analyte sensor coupled with sensor electronics and can be configured to transmit data indicative of an analyte level of a subject. The reader device can include a wireless communication circuitry configured to receive the data indicative of the analyte level and a glycated hemoglobin level for the subject, a non-transitory memory, at least one processor communicatively coupled to the non-transitory memory and the analyte sensor and configured to calculate a plurality of personalized glucose metrics for the subject using at least one physiological parameter and at least one of the received data indicative of the analyte level or the received glycated hemoglobin level, and display, on a display of the reader device, a report comprising a plurality of interfaces including at least two or more of the received data indicative of the analyte level, the received glycated hemoglobin level, or the calculated plurality of personalized glucose metrics, wherein the plurality of interfaces comprising the report are based on a user type.

As embodied herein, the plurality of personalized glucose metrics can include one or more of an adjusted A1c or personalized A1c, a calculated A1c, an adjusted calculated A1c, a personalized glucose, a personalized average glucose, or a personalized time in range. Further, the at least one processor can be configured to calculate a plurality of personalized glucose targets corresponding to the calculated plurality of personalized glucose metrics. The plurality of interfaces can further include the plurality of personalized glucose targets. Additionally, the plurality of personalized glucose targets can include one or more of a target glucose range or a target average glucose. As embodied herein, the personalized target glucose range can include a personalized lower glucose limit. Alternatively, the personalized target glucose range can include a personalized upper glucose limit.

As embodied herein, the at least one physiological parameter can be selected from the group consisting of: a red blood cell glucose uptake, a red blood cell lifespan, a red blood cell glycation rate constant, a red blood cell generation rate constant, a red blood cell elimination constant, and an apparent glycation constant. Further, the plurality of interfaces can include the at least one physiological parameter for the user.

As embodied herein, the user type can include a health care professional. Further, the plurality of interfaces can include a glucose monitoring data interface, a glycated hemoglobin interface, a personalized A1c interface, a personalized glucose interface, a personalized average glucose, and a personalized time in range interface.

As embodied herein, the user type can include the user. Further, the plurality of interfaces can include a glucose monitoring data interface, a glycated hemoglobin interface, a mean glucose interface, and a time in range interface.

As embodied herein, the plurality of interfaces comprising the report can be predetermined based on the user type.

As embodied herein, the plurality of interfaces comprising the report can be selected by the user.

As embodied herein, the at least one processor can be further configured to output a notification if at least one of the plurality of personalized glucose metrics is at or above the corresponding plurality of personalized glucose targets. As embodied herein, the notification can be a visual notification. Alternatively, the notification can be an audio notification. The notification can also be an alarm. As embodied herein, the notification can be a prompt.

As embodied herein, the reader device can wirelessly receive the glycated hemoglobin level for the subject from an electronic medical records system.

As embodied herein, the reader device can wirelessly receive the glycated hemoglobin level for the subject from a cloud-based database.

As embodied herein, the reader device can wirelessly receive the glycated hemoglobin level for the subject from a QR code.

As embodied herein, the reader device can wirelessly receive the glycated hemoglobin level for the subject from a home test kit.

The achieve these and other advantages and in accordance with the purpose of the disclosed subject matter, as embodied and broadly described, the disclosed subject matter is also directed to a method for monitoring glucose. According to an embodiment, a method for monitoring glucose can include receiving, using one or more processors, a first record including a first data associated with a personal identification from a first database, and a second record including a second data associated with a user identification from a second database. The method can further include using the one or more processors to pair the first data and the second data based upon a shared data item contained in the first record and the second record and using the one or more processors to display a report based upon the first data and the second data.

As embodied herein, the first database can be an electronic medical record system and the first data can be laboratory measured HbA1c. The second database can include an analyte monitoring system data service and the second data can include glucose levels measured by an analyte monitoring system.

As embodied herein, the shared data item can include an email address.

As embodied herein, the method can include using the one or more processors to generate a notification based upon the first data paired with the second data. In some embodiments, the method can include calculating, using the one or more processors, a plurality of personalized glucose metrics using at least one physiological parameter and at least one of the first record or the second record, wherein the first record can be laboratory measured HbA1c and the second record can be glucose level data indicative of an analyte level of a user, the report can include a plurality of interfaces including two or more of: the first record, the second record, or the calculated plurality of personalized glucose metrics, and the plurality of interfaces comprising the report can be based on a user type.

In some embodiments, the plurality of personalized glucose metrics can include one or more of an adjusted A1c, a calculated A1c, an adjusted calculated A1c, a personalized glucose, a personalized average glucose, or a personalized time in range.

In some embodiments, the method can include calculating a plurality of personalized glucose targets corresponding to the calculated plurality of personalized glucose metrics.

In some embodiments, the plurality of interfaces can include the plurality of personalized glucose targets. Further, the plurality of personalized glucose targets can include one or more of a target glucose range or a target average glucose. In other embodiments, the personalized target glucose can range include a personalized lower glucose limit. In yet another embodiment, the personalized target glucose range can include a personalized upper glucose limit.

In some embodiments, the at least one physiological parameter can be selected from the group consisting of: a red blood cell glucose uptake, a red blood cell lifespan, a red blood cell glycation rate constant, a red blood cell generation rate constant, a red blood cell elimination constant, and an apparent glycation constant.

In some embodiments, the plurality of interfaces can include the at least one physiological parameter for the user.

In some embodiments, the user type can include a heath care professional. In other embodiments, the user type can be the user. In some embodiments, the plurality of interfaces comprising the report can be predetermined based on the user type. Further, the plurality of interfaces comprising the report can be selected by the user.

In some embodiments, the plurality of interfaces can include a glucose monitoring data interface, a glycated hemoglobin interface, a personalized A1c interface, a personalized glucose interface, a personalized average glucose, and a personalized time in range interface. In other embodiments, the plurality of interfaces can include a glucose monitoring data interface, a glycated hemoglobin interface, a mean glucose interface, and a time in range interface.

In some embodiments, the method can further include outputting a notification if at least one of the plurality of personalized glucose metrics is at or above the corresponding plurality of personalized glucose target. In some embodiments, the notification can include a visual notification. In other embodiments, the notification can include an audio notification. Further, the notification can be an alarm. In some embodiments, the notification can be a prompt.

In some embodiments, the method can include receiving, at the reader device, the glycated hemoglobin level for the user from an electronic medical records system. In other embodiments, the method can include receiving, at the reader device, the glycated hemoglobin level for the user from a cloud-based database. Further, the method can include receiving, at the reader device, the glycated hemoglobin level for the user from a QR code.

In some embodiments, the method can include receiving, at the reader device, the glycated hemoglobin level for the user from an at home test kit.

### BRIEF DESCRIPTION OF THE FIGURES

The details of the subject matter set forth herein, both as to its structure and operation, may be apparent by study of the accompanying figures, in which like reference numerals refer to like parts. The components in the figures are not necessarily to scale, emphasis instead being placed upon illustrating the principles of the subject matter. Moreover, all illustrations are intended to convey concepts, where relative sizes, shapes and other detailed attributes may be illustrated schematically rather than literally or precisely.

FIG. 1 is a system overview of an analyte monitoring system comprising a sensor applicator, a sensor control device, a reader device, a network, a trusted computer system, and a local computer system.

FIG. 2A is a block diagram depicting an example embodiment of a reader device.

FIGS. 2B and 2C are block diagrams depicting example embodiments of sensor control devices.

FIG. 3 illustrates an example timeline 100 illustrating collection of at least one HbA1c value and a plurality of glucose levels for a time period.

FIG. 4 illustrates an example of a physiological parameter analysis system for providing physiological parameter analysis in accordance with some of the embodiments of the present disclosure.

FIG. 5 illustrates an example of a physiological parameter analysis system for providing physiological parameter analysis in accordance with some of the embodiments of the present disclosure.

FIG. 6 illustrates an example of a calculated HbA1c (eHbA1c) report that may be generated as an output by a physiological parameter analysis system in accordance with some of the embodiments of the present disclosure.

FIG. 7A illustrates an example of a method of determining a personalized-target glucose range in accordance with some of the embodiments of the present disclosure.

FIG. 7B illustrates an example of a personalized-target glucose range report that may be generated as an output by a physiological parameter analysis system in accordance with some of the embodiments of the present disclosure.

FIG. 8 illustrates an example of a personalized-target average glucose report that may be generated as an output by a physiological parameter analysis system in accordance with some of the embodiments of the present disclosure.

FIGS. 9A-C illustrate a comparison between the laboratory HbA1c levels at day 200 (±5 days) relative to the estimated HbA1c (eHbA1c) values for two different models (18A and 18B) and calculated HbA1c (cHbA1c) values for the kinetic model of the present disclosure (18C).

FIG. 10 illustrates an example study subject's data with the measured glucose levels (solid line), laboratory HbA1c readings (open circles), cHbA1c model values (long dashed line), and 14-day eHbA1c model values (dotted line).

FIG. 11 illustrates the relationship between steady glucose and equilibrium HbA1c (1) as determined using the standard conversion of HbA1c to estimated average glucose (dashed line with error bars) and (2) as measured for the 90 participants (solid lines).

FIG. 12 illustrates the relationship between K (dL/mg) and mean glucose level target (mg/dl) for varying HbA1c target values using the kinetic model of the present disclosure.

FIG. 13 is a graphical representation of mean glucose and laboratory A1c.

FIG. 14 provides case examples embodiments of reports of the present disclosure.

FIGS. 15A-15C are example embodiments of systems for bi-directional communication of patient data.

FIG. 16 illustrates an example environment and dataflow for an analyte monitoring system.

FIG. 17 illustrates an example dataflow for an analyte monitoring system.

FIG. 18 illustrates example user interfaces of an application executing according to certain embodiments.

FIG. 19 illustrates example user interfaces of another device according to certain embodiments.

FIG. 20 illustrates a method for requesting and generating a report based on analyte data provided via a matrix barcode according to certain embodiments.

FIG. 21 illustrates a method for requesting and generating a report based on analyte data provided via a matrix barcode according to certain embodiments.

FIG. 22 illustrates example interfaces of an application executing according to certain embodiments.

FIG. 23 illustrates example interface of an application executing according to certain embodiments.

FIG. 24 illustrates an example flow of data between the components of an analyte monitoring system according to certain embodiments.

FIG. 25 illustrates an example interface of an application executing to certain embodiments.

FIG. 26 illustrates example interfaces of an application executing according to certain embodiments.

FIG. 27 illustrates an example interface of an application executing to certain embodiments.

FIG. 28 illustrates an example interface of an application executing to certain embodiments.

FIGS. 29A-F are example embodiments of GUIs comprising sensor results interfaces.

FIGS. 29G-I are example embodiments of GUIs comprising glucose monitoring data interface and calculated A1c interfaces.

FIGS. 30A-F are example embodiments of GUIs comprising time-in-ranges interfaces.

FIGS. 31A-O are example embodiments of GUIs comprising analyte level and trend alert interfaces.

FIGS. 32A and 32B are example embodiments of GUIs comprising sensor usage interfaces.

FIGS. 32C-F are example embodiments of report GUIs including sensor usage information.

FIGS. 33-38 provide case examples embodiments of reports of the present disclosure.

### DETAILED DESCRIPTION

Before the present subject matter is described in detail, it is to be understood that this disclosure is not limited to the particular embodiments described, as such may, of course, vary. It is also to be understood that the terminology used herein is for the purpose of describing particular embodiments only, and is not intended to be limiting, since the scope of this disclosure will be limited only by the appended claims.

As used herein and in the appended claims, the singular forms “a,” “an,” and “the” include plural referents unless the context clearly dictates otherwise.

The publications discussed herein are provided solely for their disclosure prior to the filing date of this application. Nothing herein is to be construed as an admission that this disclosure is not entitled to antedate such publication by virtue of prior disclosure. Further, the dates of publication provided may be different from the actual publication dates which may need to be independently confirmed.

Generally, embodiments of this disclosure include GUIs and digital interfaces for analyte monitoring systems, and methods and devices relating thereto. Accordingly, many embodiments include in vivo analyte sensors structurally configured so that at least a portion of the sensor is, or can be, positioned in the body of a user to obtain information about at least one analyte of the body. It should be noted, however, that the embodiments disclosed herein can be used with in vivo analyte monitoring systems that incorporate in vitro capability, as well as purely in vitro or ex vivo analyte monitoring systems, including systems that are entirely non-invasive.

Furthermore, for each and every embodiment of a method disclosed herein, systems and devices capable of performing each of those embodiments are covered within the scope of this disclosure. For example, embodiments of sensor control devices, reader devices, local computer systems, and trusted computer systems are disclosed, and these devices and systems can have one or more sensors, analyte monitoring circuits (e.g., an analog circuit), memories (e.g., for storing instructions), power sources, communication circuits, transmitters, receivers, processors and/or controllers (e.g., for executing instructions) that can perform any and all method steps or facilitate the execution of any and all method steps.

As previously described, a number of embodiments described herein provide for improved GUIs for analyte monitoring systems, wherein the GUIs are highly intuitive, user-friendly, and provide for rapid access to physiological information of a user. According to some embodiments, a Time-in-Ranges GUI of an analyte monitoring system is provided, wherein the Time-in-Ranges GUI comprises a plurality of bars or bar portions, wherein each bar or bar portion indicates an amount of time that a user's analyte level is within a predefined analyte range correlating with the bar or bar portion. According to another embodiment, an Analyte Level/Trend Alert GUI of an analyte monitoring system is provided, wherein the Analyte Level/Trend Alert GUI comprises a visual notification (e.g., prompts, alert, alarm, pop-up window, banner notification, etc.), and wherein the visual notification includes an alarm condition, an analyte level measurement associated with the alarm condition, and a trend indicator associated with the alarm condition. In sum, these embodiments provide for a robust, user-friendly interfaces that can increase user engagement with the analyte monitoring system and provide for timely and actionable responses by the user, to name a few advantages.

In addition, a number of embodiments described herein provide for improved digital interfaces for analyte monitoring systems. According to some embodiments, improved methods, as well as systems and device relating thereto, are provided for data backfilling, aggregation of disconnection and reconnection events for wireless communication links, expired or failed sensor transmissions, merging data from multiple devices, transitioning of previously activated sensors to new reader devices, generating sensor insertion failure system alarms, and generating sensor termination system alarms. Collectively and individually, these digital interfaces improve upon the accuracy and integrity of analyte data being collected by the analyte monitoring system, the flexibility of the analyte monitoring system by allowing users to transition between different reader devices, and the alarming capabilities of the analyte monitoring system by providing for more robust inter-device communications during certain adverse conditions, to name only a few. Other improvements and advantages are provided as well. The various configurations of these devices are described in detail by way of the embodiments which are only examples.

Before describing these aspects of the embodiments in detail, however, it is first desirable to describe examples of devices that can be present within, for example, an in vivo analyte monitoring system, as well as examples of their operation, all of which can be used with the embodiments described herein.

There are various types of in vivo analyte monitoring systems. “Continuous Analyte Monitoring” systems (or “Continuous Glucose Monitoring” systems), for example, can transmit data from a sensor control device to a reader device continuously without prompting, e.g., automatically according to a schedule. “Flash Analyte Monitoring” systems (or “Flash Glucose Monitoring” systems or simply “Flash” systems), as another example, can transfer data from a sensor control device in response to a scan or request for data by a reader device, such as with a Near Field Communication (NFC) or Radio Frequency Identification (RFID) protocol. In vivo analyte monitoring systems can also operate without the need for finger stick calibration.

In vivo analyte monitoring systems can be differentiated from “in vitro” systems that contact a biological sample outside of the body (or “ex vivo”) and that typically include a meter device that has a port for receiving an analyte test strip carrying bodily fluid of the user, which can be analyzed to determine the user's blood sugar level.

In vivo monitoring systems can include a sensor that, while positioned in vivo, makes contact with the bodily fluid of the user and senses the analyte levels contained therein. The sensor can be part of the sensor control device that resides on the body of the user and contains the electronics and power supply that enable and control the analyte sensing. The sensor control device, and variations thereof, can also be referred to as a “sensor control unit,” an “on-body electronics” device or unit, an “on-body” device or unit, or a “sensor data communication” device or unit, to name a few.

In vivo monitoring systems can also include a device that receives sensed analyte data from the sensor control device and processes and/or displays that sensed analyte data, in any number of forms, to the user. This device, and variations thereof, can be referred to as a “handheld reader device,” “reader device” (or simply a “reader”), “handheld electronics” (or simply a “handheld”), a “portable data processing” device or unit, a “data receiver,” a “receiver” device or unit (or simply a “receiver”), or a “remote” device or unit, to name a few. Other devices such as personal computers have also been utilized with or incorporated into in vivo and in vitro monitoring systems.

### Example Embodiment of In Vivo Analyte Monitoring System

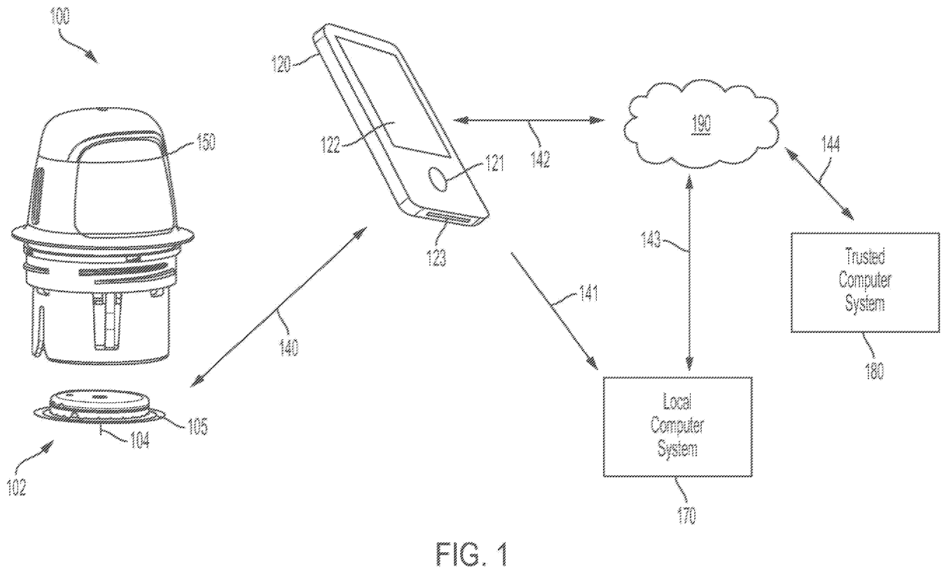

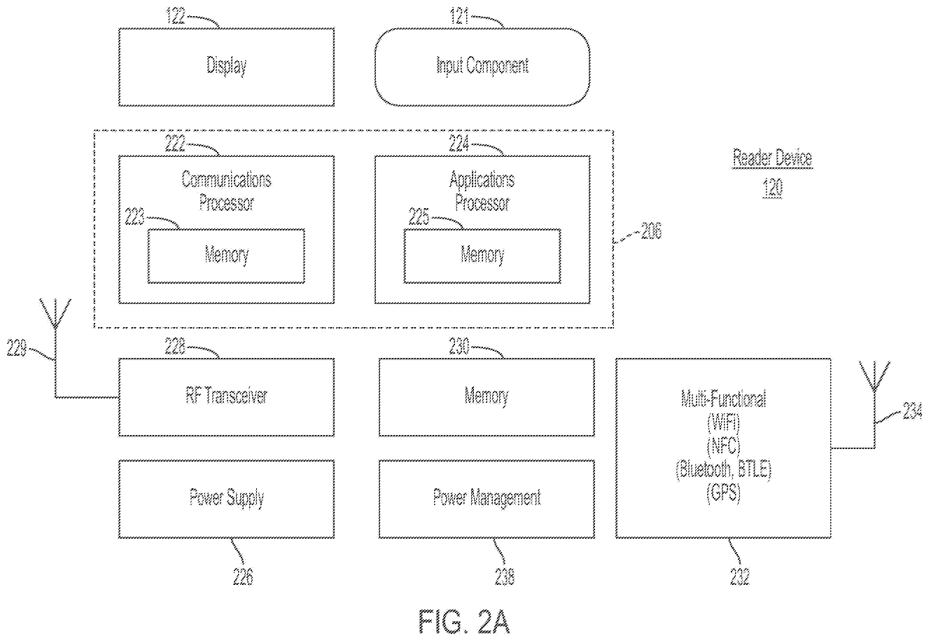

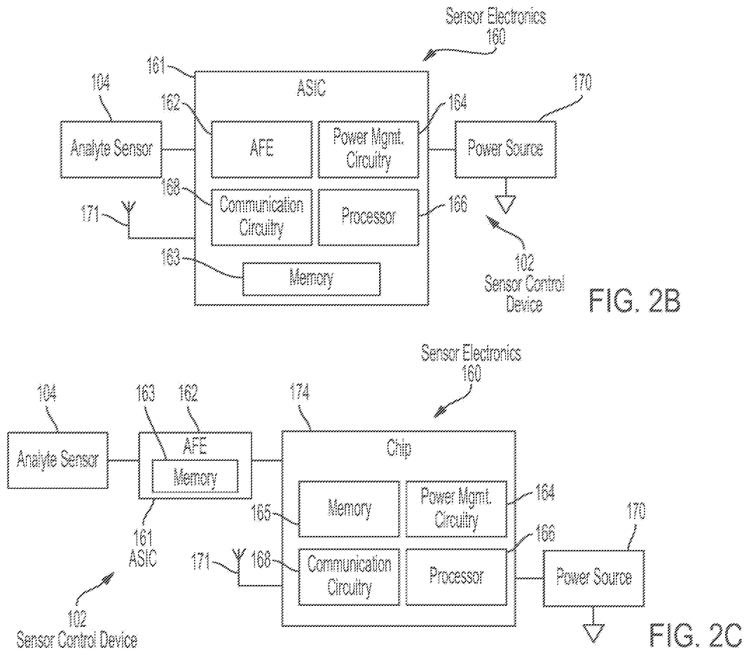

FIG. 1 is a conceptual diagram depicting an example embodiment of an analyte monitoring system 100 that includes a sensor applicator 150, a sensor control device 102, and a reader device 120. Here, sensor applicator 150 can be used to deliver sensor control device 102 to a monitoring location on a user's skin where a sensor 104 is maintained in position for a period of time by an adhesive patch 105. Sensor control device 102 is further described in FIGS. 2B and 2C, and can communicate with reader device 120 via a communication path 140 using a wired or wireless technique. Example wireless protocols include Bluetooth, Bluetooth Low Energy (BLE, BTLE, Bluetooth SMART, etc.), Near Field Communication (NFC) and others. Users can view and use applications installed in memory on reader device 120 using screen 122 (which, in many embodiments, can comprise a touchscreen), and input 121. A device battery of reader device 120 can be recharged using power port 123. While only one reader device 120 is shown, sensor control device 102 can communicate with multiple reader devices 120. Each of the reader devices 120 can communicate and share data with one another. More details about reader device 120 is set forth with respect to FIG. 2A below. Reader device 120 can communicate with local computer system 170 via a communication path 141 using a wired or wireless communication protocol. Local computer system 170 can include one or more of a laptop, desktop, tablet, phablet, smartphone, set-top box, video game console, or other computing device and wireless communication can include any of a number of applicable wireless networking protocols including Bluetooth, Bluetooth Low Energy (BTLE), Wi-Fi or others. Local computer system 170 can communicate via communications path 143 with a network 190 similar to how reader device 120 can communicate via a communications path 142 with network 190, by a wired or wireless communication protocol as described previously. Network 190 can be any of a number of networks, such as private networks and public networks, local area or wide area networks, and so forth. A trusted computer system 180 can include a cloud-based platform or server, and can provide for authentication services, secured data storage (e.g., storage of analyte measurement data received from reader device), report generation, and can communicate via communications path 144 with network 190 by wired or wireless technique. In addition, although FIG. 1 depicts trusted computer system 180 and local computer system 170 communicating with a single sensor control device 102 and a single reader device 120, it will be appreciated by those of skill in the art that local computer system 170 and/or trusted computer system 180 are each capable of being in wired or wireless communication with a plurality of reader devices and sensor control devices. Although reference is made to reader devices 120, analyte monitoring system can additionally or alternatively include multi-purpose data receiving devices 130 and user devices 140, such as those described below, including general purpose computing devices.

Additional details of suitable analyte monitoring devices, systems, methods, components and the operation thereof along with related features are set forth in U.S. Pat. No. 9,913,600 to Taub et. al., International Publication No. WO2018/136898 to Rao et. al., International Publication No. WO2019/236850 to Thomas et. al., and U.S. Patent Publication No. 2020/0196919 to Rao et al., each of which is incorporated by reference in its entirety herein.

### Example Embodiment of Reader Device

FIG. 2A is a block diagram depicting an example embodiment of a reader device 120, which, in some embodiments, can comprise a smart phone or a smart watch. Here, reader device 120 can include a display 122, input component 121, and a processing core 206 including a communications processor 222 coupled with memory 223 and an applications processor 224 coupled with memory 225. Also included can be separate memory 230, RF transceiver 228 with antenna 229, and power supply 226 with power management module 238. Further, reader device 120 can also include a multi-functional transceiver 232, which can comprise wireless communication circuitry, and which can be configured to communicate over Wi-Fi, NFC, Bluetooth, BTLE, and GPS with one or more antenna 234. As understood by one of skill in the art, these components are electrically and communicatively coupled in a manner to make a functional device.

### Example Embodiments of Sensor Control Devices

FIGS. 2B and 2C are block diagrams depicting example embodiments of sensor control devices 102 having analyte sensors 104 and sensor electronics 160 (including analyte monitoring circuitry) that can have the majority of the processing capability for rendering end-result data suitable for display to the user. In FIG. 2B, a single semiconductor chip 161 is depicted that can be a custom application specific integrated circuit (ASIC). Shown within ASIC 161 are certain high-level functional units, including an analog front end (AFE) 162, power management (or control) circuitry 164, processor 166, and communication circuitry 168 (which can be implemented as a transmitter, receiver, transceiver, passive circuit, or otherwise according to the communication protocol). In this embodiment, both AFE 162 and processor 166 are used as analyte monitoring circuitry, but in other embodiments either circuit can perform the analyte monitoring function. Processor 166 can include one or more processors, microprocessors, controllers, and/or microcontrollers, each of which can be a discrete chip or distributed amongst (and a portion of) a number of different chips.

A memory 163 is also included within ASIC 161 and can be shared by the various functional units present within ASIC 161, or can be distributed amongst two or more of them. Memory 163 can also be a separate chip. Memory 163 can be volatile and/or non-volatile memory. In this embodiment, ASIC 161 is coupled with power source 170, which can be a coin cell battery, or the like. AFE 162 interfaces with in vivo analyte sensor 104 and receives measurement data therefrom and outputs the data to processor 166 in digital form, which in turn processes the data to arrive at the end-result glucose discrete and trend values, etc. This data can then be provided to communication circuitry 168 for sending, by way of antenna 171, to reader device 120 (not shown), for example, where minimal further processing is needed by the resident software application to display the data. According to some embodiments, for example, a current glucose value can be transmitted from sensor control device 102 to reader device 120 every minute, and historical glucose values can be transmitted from sensor control device 102 to reader device 120 every five minutes.

In some embodiments, to conserve power and processing resources on sensor control device 102, digital data received from AFE 162 can be sent to reader device 120 (not shown) with minimal or no processing. In still other embodiments, processor 166 can be configured to generate certain predetermined data types (e.g., current glucose value, historical glucose values) either for storage in memory 163 or transmission to reader device 120 (not shown), and to ascertain certain alarm conditions (e.g., sensor fault conditions), while other processing and alarm functions (e.g., high/low glucose threshold alarms) can be performed on reader device 120. Those of skill in the art will understand that the methods, functions, and interfaces described herein can be performed—in whole or in part—by processing circuitry on sensor control device 102, reader device 120, local computer system 170, or trusted computer system 180.

FIG. 2C is similar to FIG. 2B but instead includes two discrete semiconductor chips 162 and 174, which can be packaged together or separately. Here, AFE 162 is resident on ASIC 161. Processor 166 is integrated with power management circuitry 164 and communication circuitry 168 on chip 174. AFE 162 may include memory 163 and chip 174 includes memory 165, which can be isolated or distributed within. In one example embodiment, AFE 162 is combined with power management circuitry 164 and processor 166 on one chip, while communication circuitry 168 is on a separate chip. In another example embodiment, both AFE 162 and communication circuitry 168 are on one chip, and processor 166 and power management circuitry 164 are on another chip. It should be noted that other chip combinations are possible, including three or more chips, each bearing responsibility for the separate functions described, or sharing one or more functions for fail-safe redundancy.

### Example Embodiments of Graphical User Interfaces for Analyte Monitoring Systems

Described herein are example embodiments of GUIs for analyte monitoring systems. As an initial matter, it will be understood by those of skill in the art that the GUIs described herein comprise instructions stored in a memory of reader device 120, local computer system 170, trusted computer system 180, and/or any other device or system that is part of, or in communication with, analyte monitoring system 100. These instructions, when executed by one or more processors of the reader device 120, local computer system 170, trusted computer system 180, or other device or system of analyte monitoring system 100, cause the one or more processors to perform the method steps and/or output the GUIs described herein. Those of skill in the art will further recognize that the GUIs described herein can be stored as instructions in the memory of a single centralized device or, in the alternative, can be distributed across multiple discrete devices in geographically dispersed locations.

### Example Embodiments of Models for Personalized Glucose-Related Metrics

Described herein are example embodiments of exemplary embodiments of models for personalized glucose-related metrics. The present disclosure generally describes methods, devices, and systems for determining physiological parameters related to the kinetics of red blood cell glycation, elimination, and generation and reticulocyte maturation within the body of a subject. Such physiological parameters can be used, for example, to calculate a more reliable calculated HbA1c (cHbA1c), adjusted or personalized HbA1c (aHbA1c), adjusted calculated HbA1c (acHbA1c), and/or a personalized target glucose range, among other things, for subject-personalized diagnoses, treatments, and/or monitoring protocols.

Herein, the terms “HbA1c level,” “HbA1c value,” and “HbA1c” are used interchangeably. Herein, the terms “personalized A1c,” “personalized HbA1c,” “aHbA1c level,” “aHbA1c value,” and “aHbA1c” are used interchangeably. Herein, the terms “cHbA1c level,” “cHbA1c value,” “cHbA1c,” and “GD-A1c” are used interchangeably and/or a personalized target glucose range, among other things. Herein, the terms “acHbA1c level,” “acHbA1c value,” and “acHbA1c,” are used interchangeably.

Kinetic Model

High glucose exposure in specific organs (particularly eye, kidney and nerve) is a critical factor for the development of diabetes complications. A laboratory HbA1c (also referred to in the art as a measured HbA1c) is routinely used to assess glycemic control, but studies report a disconnect between this glycemic marker and diabetes complications in some individuals. The exact mechanisms for the failure of laboratory HbA1c to predict diabetes complications are not often clear but likely in some cases to be related to inaccurate estimation of intracellular glucose exposure in the affected organs.

Formula 1 illustrates the kinetics of red blood cell hemoglobin glycation (or referred to herein simply as red blood cell glycation), red blood cell elimination, and red blood cell generation, where “G” is free glucose, “R” is a non-glycated red blood cell, and “GR” is glycated red blood cell hemoglobin. The rate at which glycated red blood cell hemoglobin (GR) are formed is referred to herein as a red blood cell hemoglobin glycation rate constant (k_{gly} typically having units of dl_*mg-¹*day^{!}).

Over time, red blood cells including the glycated red blood cells are continuously eliminated from a subject's circulatory system and new red blood cells are generated, typically at a rate of approximately 2 million cells per second. The rates associated with elimination and generation are referred to herein as a red blood cell elimination constant (k_{age} typically having units of day⁻¹) and a red blood cell generation rate constant (k_{gen} typically having units of M²/day), respectively. Since the amount of red blood cells in the body is maintained at a stable level most of time, the ratio of k_{age} and k_{gen} should be an individual constant that is the square of red blood cell concentration.

Relative to glycation, Formula 2 illustrates the mechanism in more detail where glucose transporter 1 (GLUT1) facilitates glucose (G) transport into the red blood cell. Then, the intracellular glucose (GI) interacts with the hemoglobin (Hb) to produce glycated hemoglobin (HbG) where the hemoglobin glycation reaction rate constant is represented by k_{g} (typically having units of dl_*mg-¹*day^{!}). A typical experiment measured k_{g} value is 1.2×10³ db/mg/day. Hemoglobin glycation reaction is a multi-step non-enzymatic chemical reaction, therefore k_{g} should be a universal constant. The rate constant for the glucose to be transported into the red blood cell and glycated the Hb into HbG is kgly. Then, k_{age} describes red blood cell elimination (along with hemoglobin), also described herein as the red blood cell turnover rate.

While raised intracellular glucose is responsible for diabetes complications, extracellular hyperglycemia selectively damages cells with limited ability to adjust cross-membrane glucose transport effectively. HbA1c has been used as a biomarker for diabetes-related intracellular hyperglycemia for two main reasons. First, the glycation reaction occurs within red blood cells (RBCs) and therefore HbA1c is modulated by intracellular glucose level. Second, RBCs do not have the capacity to adjust glucose transporter GLUT1 levels and thus are unable to modify cross-membrane glucose uptake, behaving similarly to cells that are selectively damaged by extracellular hyperglycemia. Therefore, under conditions of fixed RBC lifespan and cross-membrane glucose uptake, HbA1c mirrors intracellular glucose exposure in organs affected by diabetes complications. However, given the inter-individual variability in both cross-membrane glucose uptake and RBC lifespan, laboratory HbA1c may not always reflect intracellular glucose exposure. While variation in RBC cross-membrane glucose uptake is likely to be relevant to the risk of estimating diabetes complications in susceptible organs, red blood cell lifespan is unique to RBCs and therefore irrelevant to the complication risk in other tissues. This explains the inability to clinically rely on laboratory HbA1c in those with hematological disorders characterized by abnormal RBC turnover and represents a possible explanation for the apparent “disconnect” between laboratory HbA1c and development of complications in some individuals with diabetes (FIG. 1).

To overcome the limitations of laboratory HbA1c, a measure of personalized HbA1c has been developed, which takes into account individual variations in both RBC turnover and cellular glucose uptake. The current work aims to extend this model by adjusting for a standard RBC lifespan of 100 days (equivalent to RBC turnover rate of 1% per day, or mean RBC age of 50 days) to establish a new clinical marker, which we term adjusted HbA1c (aHbA1c). We propose that aHbA1c is the most relevant glycemic marker for estimating organ exposure to hyperglycemia and risk of future diabetes-related complications

As described previously, HbA1c is a commonly used analyte indicative of the fraction of the glycated hemoglobin found in red blood cells. Therefore, a kinetic model can be used, for example, to derive a calculated HbA1c based on at least the glucose levels measured for a subject. However, the kinetic model can also be applied to HbA1. For simplicity, HbA1c is uniformly used herein, but HbA1 could be substituted except in instances where specific HbA1c values are used (e.g., see Equations 15 and 16). In such instances, specific HbA1 values could be used to derive similar equations.

Typically, when kinetically modeling physiological processes, assumptions are made to focus on the factors that affect the physiological process the most and simplify some of the math.

The present disclosure uses only the following set of assumptions to kinetically model the physiological process illustrated in Formula 1. First, glucose concentration is high enough not to be affected by the red blood cell glycation reaction. Second, there is an absence of abnormal red blood cells that would affect HbA1c measurement, so the hematocrit is constant for the period of interest. This assumption was made to exclude extreme conditions or life events that are not normally present and may adversely affect the accuracy of the model. Third, the glycation process has first order dependencies on both red blood cell and glucose concentrations. Fourth, newly-generated red blood cells have a negligible amount of glycated hemoglobin, based on previous reports that reticulocyte HbA1c is very low and almost undetectable. Fifth, red blood cell production inversely correlates with total cellular concentration, whereas elimination is a first order process.

With the five assumptions described above for this kinetic model, the rate of change in glycated and non-glycated red blood cells can be modeled by differential Equations 1 and 2.

d[GR\/dt=kgly[G][R]−k_{age}[GR] Equation 1

(d[R])/dt=k_{gen}/C−k_{age}[R]−k_{gly}[G][R] Equation 2

C is the whole population of red blood cells, where C=[ff]+[GR] (Equation 2a). C typically has units of M (mol/L), [R] and [GR] typically have units of M, and [G] typically has units of mg/dl.

Assuming a steady state, where the glucose level is constant and the glycated and non-glycated red blood cell concentrations remain stable (d[GR]/dt=(d[R])/dt=0), the following two equations can be derived. Equation 3 defines the apparent glycation constant K (typically with units of dL/mg) as the ratio of k_{gly} and k_{age}, whereas Equation 4 establishes the dependency between red blood cell generation and elimination rates.

K=k_{gly}/k_{age}=[GR]/[G][R] Equation 3

k_{gen}/k_{age}=C² Equation 4

For simplicity, k_{age} is used hereafter to describe the methods, devices, and systems of the present disclosure. Unless otherwise specified, k_{gen} can be substituted for k_{age}. To substitute k_{gen} for k_{age}, Equation 4 would be rearranged to k_{gen}−k_{age}*C.

HbA1c is the fraction of glycated hemoglobin as shown in Equation 5.

HbA1c=[GR]/C=(C−[R])/C Equation 5

In a hypothetical state when a person infinitely holds the same glucose level, HbA1c in Equation 5 can be defined as “equilibrium HbA1c” (EA) (typically reported as a % (e.g., 6.5%) but used in decimal form (e.g., 0.065) in the calculations). For a given glucose level, EA (Equation 6) can be derived from Equations 2a, 3, and 5.

EA=(k_{gly}[G])/(k_{age}+k_{gly}[G])=[G]/(K⁻¹+[G]) Equation 6

EA is an estimate of HbA1c based on a constant glucose concentration [G] for a long period. This relationship effectively approximates the average glucose and HbA1c for an individual having a stable day-to-day glucose profile. EA depends on K, the value of which is characteristic to each subject. Equation 6 indicates that the steady glucose is not linearly correlated with EA. Steady glucose and EA may be approximated with a linear function within a specific range of glucose level, but not across the full typical clinical range of HbA1c. Furthermore, in real life with continuous fluctuations of glucose levels, there is no reliable linear relationship between laboratory HbA1c and average glucose for an individual.

Others have concluded this also and produced kinetic models to correlate a measured HbA1c value to average glucose levels. For example, The American Diabetes Association has an online calculator for converting HbA1c values to estimated average glucose levels. However, this model is based on an assumption that k_{age} and k_{gly} do not substantially vary between subjects, which is illustrated to be false in Example 1 below. Therefore, the model currently adopted by the American Diabetes Association considers k_{age} and k_{gly} as constants and not variable by subject.

A more recent model by Higgens et al. (Sci. Transl. Med. 8, 359ra130, 2016) has been developed that removed the assumption that red blood cell life is constant. However, the more recent model still assumes that k_{gly} does not substantially vary between subjects.

In contrast, both k_{age} and k_{gly} are variables for the kinetic models described herein. Further, a subject's k_{gly} is used in some embodiments to derive personalized parameters relating to the subject's diabetic condition and treatment (e.g., a medication dosage, a supplement dosage, an exercise plan, a diet/meal plan, and the like).

Continuing with the kinetic model of the present disclosure, the HbA1c value (HbA1cₜ) at the end of a time period t (Equation 7) can be derived from Equation 1, given a starting HbA1c (HbA1co) and assuming a constant glucose level [G] during the time period.

HbA1cₜ=EA+(HbA1c₀−EA)*e^{-(k}^{_{gly}}^{[G]+k}^{_{age}}^{)t} Equation 7

To accommodate changing glucose levels over time, each individual's glucose history is approximated as a series of time intervals t, with corresponding average glucose levels [G,]. Applying Equation 7 recursively, HbA1C_{z} at the end of time interval t_{z} can be expressed by Equation 8 for numerical calculations.

HbA1c_{z}=EA_{z}(1−D_{z})+Σᵢ₌₁^{z-1}[EAᵢ(1−Di)Πⱼ₌ᵢ₊₁^{z}Dⱼ]+HbA1c₀Πⱼ₌₁^{z}Dⱼ

Equation 8 where the decay term Dₜ=ₑ−(y^{[G;]+/c}agₑ)^{t}£ (Equation 8a).

When solving for k_{age} and k_{gly} using Equations 6, 7, or 8, k_{age} and k_{gly} may be bounded to reasonable physiological limits, by way of nonlimiting example, of 5.0*10⁶ dl_*mg\hat{ }day-¹<k_{gly}<8.0*10⁶ dl_*mg\hat{ }day¹ and 0.006 day¹<k_{age}<0.024 day⁻¹. Additionally or alternatively, an empirical approach using the Broyden-Fletcher-Goldfarb-Shanno algorithm can be used with estimated initial values for k_{gly} and k_{age} (e.g., k_{gly}=4.4*10⁻⁶ dl_*mg\hat{ }day¹ and k_{age}=0.0092 day X). The more glucose level data points and measured HbA1c data points, the more accurate the physiological parameters described herein are.

The value for time interval t, can be selected (e.g., by a user or developer, or by software instructions being executed on one or more processors) based on a number of factors that can vary between embodiments and, as such, the value of time interval t may vary. One such factor is the duration of time from one glucose data value (e.g., a measured glucose level at a discrete time, a value representative of glucose level for a particular time period across multiple discrete times, or otherwise) to another within the individual's glucose history. That duration of time between glucose data values can be referred to as time interval t_{g}. Time interval t_{g} can vary across the individual's glucose history such that a single glucose history can have a number of different values for time interval t_{g}. Numerous example embodiments leading to different values of time interval t_{g} are described herein. In some embodiments of glucose monitoring systems, glucose data points are determined after a fixed time interval t_{g} (e.g., every minute, every ten minutes, every fifteen minutes, etc.) and the resulting glucose history is a series of glucose data points with each point representing the glucose at the expiration of or across the fixed time interval t_{g} (e.g., a series of glucose data points at one minute intervals, etc.). [0037] In other embodiments, glucose data points are taken or determined at multiple different fixed time intervals t_{g}. For example, in some flash analyte monitoring systems (described in further detail herein), a user may request glucose data from a device (e.g., a sensor control device) that stores glucose data within a recent time period (e.g., the most recent fifteen minutes, the most recent hour, etc.) at a first relatively shorter time interval t_{g} (e.g., every minute, every two minutes), and all other data (in some cases up to a maximum of eight hours, twelve hours, twenty-four hours, etc.) outside of that recent time period is stored at a second relatively longer time interval t_{g} (e.g., every ten minutes, every fifteen minutes, every twenty minutes, etc.). The data stored at the second, relatively longer time interval can be determined from data originally taken at the relatively shorter time interval t_{g} (e.g., an average, median, or other algorithmically determined value). In such an example the resulting glucose history is dependent on how often a user requests glucose data, and can be a combination of some glucose data points at the first time interval t_{g} and others at the second time interval t_{g}. Of course, more complex variations are also possible with, for example, three or more time intervals t_{g}. In some embodiments, glucose data collected with ad hoc adjunctive measurements (e.g., a finger stick and test strip) can also be present, which can result in even more variations of time interval t_{g}.

An example analysis performed on glucose histories for a sample of subjects (approximately 400) where glucose data points were generally present at time intervals t_{g} of one to fifteen minutes, indicated that a value for time interval t, within the range of three hours (or about three hours) to twenty four hours (or about twenty four hours) could be selected without significant loss of accuracy. Generally, shorter time intervals t, resulted in higher accuracy than longer ones, and time interval t, values closer to three hours were the most accurate. Time interval t, values less than three hours may begin to exhibit loss of accuracy due to numerical rounding errors. These rounding errors can be reduced by using longer digit strings at the expense of processing load and computing time. It should be noted that other values of time interval t, outside of the range of 3 to 24 hours may be suitable depending on the desired accuracy levels and other factors, such as the average time interval t_{g} between glucose data points.

Another factor in selection of time interval t, is the existence of gaps, or missing data, in the individual's glucose history, where the gaps are longer or significantly longer than the longest time interval t_{g}. The existence of one or more such gaps can potentially lead to results bias. These gaps can result, for example, from the inability to collect glucose data across a certain time period (e.g., the user was not wearing a sensor, the user forgot to scan the sensor for data, a fault occurred, etc.). The presence of gaps and their duration should be considered in selecting time interval t. Generally, the number and duration of gaps should be minimized (or eliminated) where possible. But since gaps of this type are often difficult to eliminate, to the extent such gaps exist, in many embodiments the selection of time interval t, should be at least twice the duration of the largest (maximum) gap between glucose data points. For example, if time interval t, is selected to be 3 hours, then the maximum gap should be no longer than 90 minutes, if time interval t, is selected to be 24 hours, then the largest gap should be no longer than 12 hours, and so forth.

The value HbA1c_{z} is the estimated HbA1c of the present kinetic model, which is referred to herein as cHbA1c (calculated HbA1c) to distinguish from other eHbA1c described herein.

As described previously and illustrated in Equation 8, EA, and D, are both affected by glucose level [G,], k_{gly}, and k_{age}. In addition, D, depends on the length of the time interval t. Equation 8 is the recursive form of Equation 7. Equations 7 and 8 describe the relationship among HbA1c, glucose level, and individual red blood cell kinetic constants k_{gly} and k_{age}.

k_{age} can be directly measured through expensive and laborious methods. Herein, the kinetic model is extended to incorporate reticulocyte maturation as a method for estimating k_{age}.

Reticulocytes are immature red blood cells and typically account for about 1% of the total red blood cells. The rate at which reticulocytes mature into mature red blood cells is kₘₐₜ (typically having units of day⁻¹). The maturation half-life for a normal reticulocyte is about 4.8 hours, which provides for Equation 9.

kₘₐₜ=ln 2/(4.8 hours)=3.47 day⁻¹ Equation 9

The kinetic model makes two assumptions: (1) all red blood cells are reticulocytes at time 0 and (2) reticulocytes are not eliminated (that is, reticulocytes mature to mature red blood cells and do not die). The probability density of reticulocyte age (PRET) can be represented by Equation 10.

p_{RET}(τ)=(k_{age}/(1−ln 2))*e^{-k}^{ₘₐₜ}^{*τ} Equation 10

where t is the cell age.

A reticulocyte production index (RPI), also known as a corrected reticulocyte count (CRC), is the percentage of total red blood cells that are reticulocytes. Therefore, RPI is the integral of PRET over cell age as shown in Equation 11, where RPI is the decimal form of the reported RPI (e.g., RPI reported at 2% is 0.02 in Equation 11).

RPI=∫₀^{∞}pRET(τ)dτ=k_{age}/(kₘₐₜ*(1−ln 2)) Equation 11

Assuming the typical kₘₐₜ is 3.47 day⁻¹, k_{age} can be estimated from a measured RPI. RPI can be determined by normal methods. For example, RPI can be determined by measuring a hematocrit percentage (HMₘ), measuring a percentage of reticulocytes (RP) in an RNA dyed blood smear, determining a maturation correction (MC) from the measured hematocrit percentage, and calculating the RPI based on Equation 12, where RP and HMₘ is used as the percentage values not the decimal form (i.e., RP reported at 3% is 3 in the equation not 0.03).

Assuming the typical kₘₐₜ is 3.47 day⁻¹, k_{age} can be estimated from a measured RPI. RPI can be determined by normal methods. For example, RPI can be determined by measuring a hematocrit percentage (HMₘ), measuring a percentage of reticulocytes (RP) in an RNA dyed blood smear, determining a maturation correction (MC) from the measured hematocrit percentage, and calculating the RPI based on Equation 12, where RP and HMₘ is used as the percentage values not the decimal form (i.e., RP reported at 3% is 3 in the equation not 0.03).

RPI=(RP*HMₘ/HMₙ)/MC Equation 12

where HMₙ is the normal hematocrit value (typically 45).

Unless otherwise specified, the typical units described are associated with their respective values. One skilled in the art would recognize other units and the proper conversions. For example, [G] is typically measured in mg/dL but could be converted to M using the molar mass of glucose. If [G] is used in M or any other variable is used with different units, the equations herein should be adjusted to account for differences in units.

Calculating Physiological Parameters from the Kinetic Model

Embodiments of the present disclosure provide kinetic modeling of red blood cell glycation, elimination, and generation and reticulocyte maturation within the body of a subject.

The physiological parameter k_{age} can be estimated from one or more RPI measurements. While k_{age} can be estimated using Equation 11 above from a single RPI measurement, two or more RPI measurements may increase the accuracy of the RPI value. Further, RPI can change over time, in response to treatment, and in response to the improvement or worsening of a disease state. Therefore, while RPI can be measured be measured in any desired intervals of time (e.g., weekly to annually), preferably RPI is measured once every three to six months.

Once k_{age} is calculated, the physiological parameters k_{gly} and/or K can be estimated from the equations described herein given at least one measured HbA1c value (also referred to as HbA1c level measurement) and a plurality of glucose levels (also referred to as glucose level measurements) over a time period immediately before the HbA1c measurement.

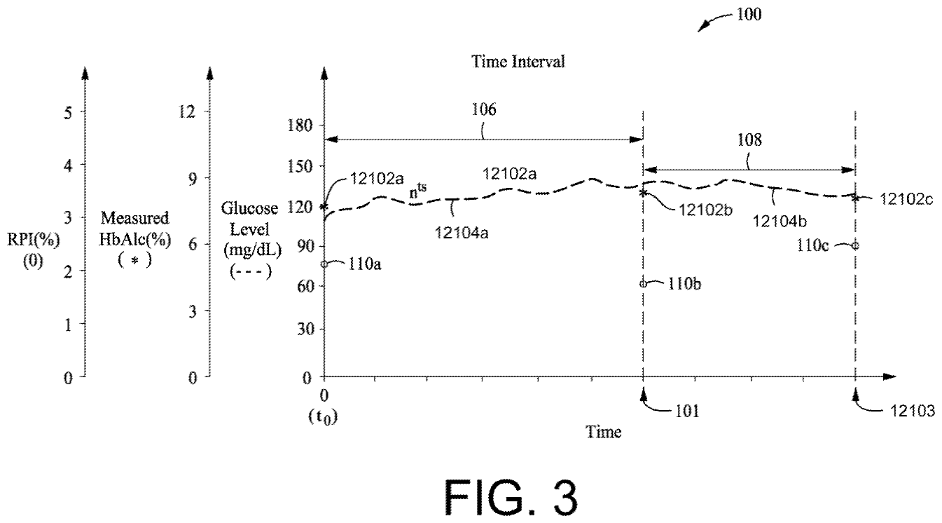

FIG. 3 illustrates an example time line 100 illustrating a collection of at least one measured HbA1c value 12102a, 12102b, 12102c, a plurality of glucose levels 12104a and 12104b, and at least one measured RPI value 110a, 110b, 110c over time periods 106 and 108.

The number of measured HbA1c values 12102a, 12102b, 12102c needed to calculate k_{gly} and/or K depends on the frequency and duration of the plurality of glucose levels. The number of measured RPI values 110a, 110b, 110c needed to calculate k_{age} depends on the stability of individual kₘₐₜ and its deviation to typical kₘₐₜ (3.47 day⁻¹). Preferably RPI is measured once every three to six months but can be measured monthly or weekly, if needed.

In a first embodiment, one measured RPI value 110b can be used to calculate k_{age}, and one measured HbA1c 12102b can be used along with the calculated k_{age} and a plurality of glucose measurements over time period 106 to calculate k_{gly} and/or K. Such embodiments are applicable to subjects with steady daily glucose measurements for a long time period 106 (e.g., over about 200 days). K may be calculated at time point 101 with Equation 6 by replacing EA with the measured HbA1c value 12102b and [G] with daily average glucose over time period 106. k_{gly} may then be calculated from Equation 3. Therefore, in this embodiment, an initial HbA1c level measurement 12102a is not necessarily required.

Because a first HbA1c value is not measured, the time interval 106 of initial glucose level measurements with frequent measurements may need to be long to obtain an accurate representation of average glucose and reduce error. Using more than 100 days of steady glucose pattern for this method may reduce error. Additional length like 200 days or more or 300 days or more further reduces error.

Embodiments where one measured HbA1c value 12102b can be used include a time period 106 about 100 days to about 300 days (or longer) with glucose levels being measured at least about 72 times per day (e.g., about every 20 minutes) to about 96 times per day (e.g., about every 15 minutes) or more often. Further, in such embodiments, the time between glucose level measurements may be somewhat consistent where an interval between two glucose level measurements should not be more than about an hour. Some missing data glucose measurements are tolerable when using only one measured HbA1c value. Increases in missing data may lead to more error.

Alternatively, in some instances where one measured HbA1c value 12102b is used, the time period 106 may be shortened if a subject has an existing glucose level monitoring history with stable, consistent glucose profile. For example, for a subject who has been testing for a prolonged time (e.g., 6 months or longer) but, perhaps, at less frequent or regimented times, the existing glucose level measurements can be used to determine and analyze a glucose profile. Then, if more frequent and regimented glucose monitoring is performed over time period 106 (e.g., about 72 times to about 96 times or more per day over about 14 days or more) followed by measurement of HbA1c 12102b and RPI 110b, the four sets of data in combination may be used to calculate one or more physiological parameters (k_{gly}, k_{age}, and/or K) at time point 101.

Alternatively, in some embodiments, one or more measured RPI values 110a, 110b, two measured HbA1c values (a first measured HbA1c value 12102a at the beginning of a time period 106 and a second measured HbA1c value 12102b at the end of the time period 106), and a plurality of glucose levels 12104a measured during the time period 106 may be used to calculate one or more physiological parameters (k_{gly}, k_{age}, and/or K) at time point 101. In these embodiments, Equation 11 may be used to calculate k_{age}, and Equation 8 may be used to calculate k_{gly} and/or K at time point 101. In such embodiments, the plurality of glucose levels 12104a may be measured for about 10 days to about 30 days or longer with measurements being, on average, about 4 times daily (e.g., about every 6 hours) to about 24 times daily (e.g., about every 1 hour) or more often.

In the foregoing embodiments, the RPI value(s) can be measured at a time other than as illustrated because measured RPI values are relatively stable over time. Therefore, the RPI value(s) can be measured at any time during time period 106 and be applicable to these embodiments.

The foregoing embodiments are not limited to the example glucose level measurement time period and frequency ranges provided. Glucose levels may be measured over a time period of about a few days to about 300 days or more (e.g., about one week or more, about 10 days or more, about 14 days or more, about 30 days or more, about 60 days or more, about 90 days or more, about 120 days or more, and so on). In some embodiments, the time period is 7 days or more, preferably one to ten months, and less than one year. The frequency of such glucose levels may be, on average, about 14,400 times daily (e.g., a time interval t_{g} of about every 6 seconds) (or more often) to about 3 times daily (e.g., a time interval t_{g} of about every 8 hours) (e.g., 1,440 times daily (e.g., a time interval t_{g} of about every minute), about 288 times daily (e.g., a time interval t_{g} of about every 5 minutes), about 144 times daily (e.g., a time interval t_{g} of about every 10 minutes), about 96 times daily (e.g., a time interval t_{g} of about every 15 minutes), about 72 times daily (e.g., a time interval t_{g} of about every 20 minutes), about 48 times daily (e.g., a time interval t_{g} of about every 30 minutes), about 24 times daily (e.g., a time interval t_{g} of about every 1 hour), about 12 times daily (e.g., a time interval t_{g} of about every 2 hours), about 8 times daily (e.g., a time interval t_{g} of about every 3 hours), about 6 times daily (e.g., a time interval t_{g} of about every 4 hours), about 4 times daily (e.g., a time interval t_{g} of about every 6 hours), and so on). In some instances, less frequent monitoring (like once or twice daily) may be used where the glucose measurements occur at about the same time (within about 30 minutes) daily to have a more direct comparison of day-to-day glucose levels and reduce error in subsequent analyses.

The foregoing embodiments may further include calculating an error or uncertainty associated with the one or more physiological parameters. In some embodiments, the error may be used to determine if another HbA1c value (not illustrated) should be measured near time point 101, if one or more glucose levels 12104b should be measured (e.g., near time point 101), if the monitoring and analysis should be extended (e.g., to extend through time period 108 from time point 101 to time point 12103 including measurement of glucose levels 12104b during time period 108 and measurement of HbA1c value 12102c at time point 12103), and/or if the frequency of glucose level measurements 12104b in an extended time period 108 should be increased relative to the frequency of glucose level measurements 12104a during time period 106. In some embodiments, one or more of the foregoing actions may be taken when the error associated with k_{gly}, k_{age}, and/or K is at or greater than about 15%, preferably at or greater than about 10%, preferably at or greater than about 7%, and preferably at or greater than about 5%. When a subject has an existing disease condition (e.g., cardiovascular disease), a lower error may be preferred to have more stringent monitoring and less error in the analyses described herein.

Alternatively or when the error is acceptable, in some embodiments, one or more physiological parameters (k_{gly}, k_{age}, and/or K) at time point 101 may be used to determine one or more parameters or characteristics for a subject's personalized diabetes management (e.g., a cHbA1c at the end of time period 108, a personalized-target glucose range, and/or a treatment or change in treatment for the subject in the near future), each described in more detail further herein. In some instances, in addition to the foregoing embodiments, an HbA1c value may be measured at time point 12103 and the one or more physiological parameters recalculated and applied to a future time period (not illustrated).

Alternatively or additionally, two values for k_{age} can be estimated using Equation 8 and Equation 11. A comparison of these two values can be used to determine if another HbA1c value (not illustrated) should be measured near time point 101, if one or more glucose levels 12104b should be measured (e.g., near time point 101), if the monitoring and analysis should be extended (e.g., to extend through time period 108 from time point 101 to time point 12103 including measurement of glucose levels 12104b and measurement of HbA1c value 12102c at time point 12103), and/or if the frequency of glucose level measurements 12104b in an extended time period 108 should be increased relative to the frequency of glucose level measurements 12104a during time period 106. For example, if the two values of k_{age} are more than 10% different (e.g., the low value is not within 10% of the high value based on the high value), the individual's kₘₐₜ may be different than the typical kmat (3.47 day⁻¹). If a large difference is observed (e.g., more than 20% difference), the individual's kmat could be determined. If the individual's kmat is stable over a time period (e.g., three to six months), the determined individual's kmat should be used in place of the typical kmat in Equation 11 in the methods, systems, and devices described herein. Fluctuation in kmat could suggest other health problems.

The one or more physiological parameters and/or the one or more parameters or characteristics for a subject's personalized diabetes management can be measured and/or calculated for two or more times (e.g., time point 101 and time point 12103) and compared. For example, k_{gly} at time point 101 and time point 12103 may be compared. In another example, cHbA1c at time point 12103 and at a future time may be compared. Some embodiments, described further herein, may use such comparisons to (1) monitor progress and/or effectiveness of a subject's personalized diabetes management and, optionally, alter the subject's personalized diabetes management, (2) identify an abnormal or diseased physiological condition, and/or (3) identify subjects taking supplements and/or medicines that affect red blood cell production and/or affect metabolism.

Each of the example methods, devices, and systems described herein can utilize the one or more physiological parameters (k_{gly}, k_{age}, and K) and perform one or more related analyses (e.g., personalized-target glucose range, personalized-target average glucose, cHbA1c, and the like). The one or more physiological parameters (k_{gly}, k_{age}, and K) and related analyses may be updated periodically (e.g., about every 3 months to annually). The frequency of updates may depend on, among other things, the subject's glucose level and diabetes history (e.g., how well the subject stays within the prescribed thresholds), other medical conditions, and the like.

Other Factors

In the embodiments described herein that apply the one or more physiological parameters (k_{gly}, k_{age}, and/or K), one or more other subject-specific parameters may be used in addition to the one or more physiological parameters. Examples of subject-specific parameters may include, but are not limited to, vital information (e.g., heart rate, body temperature, blood pressure, or any other vital information), body chemistry information (e.g., drug concentration, blood levels, troponin level, cholesterol level, or any other body chemistry information), meal data/information (e.g., carbohydrate amount, sugar amount, or any other information about a meal), activity information (e.g., the occurrence and/or duration of sleep and/or exercise), an existing medical condition (e.g., cardiovascular disease, heart valve replacement, cancer, and systemic disorder such as autoimmune disease, hormone disorders, and blood cell disorders), a family history of a medical condition, a current treatment, an age, a race, a gender, a geographic location (e.g., where a subject grew up or where a subject currently lives), a diabetes type, a duration of diabetes diagnosis, and the like, and any combination thereof.

Systems

In some embodiments, determining the one or more physiological parameters (k_{gly}, k_{age}, and/or K) for a subject may be performed using a physiological parameter analysis system.

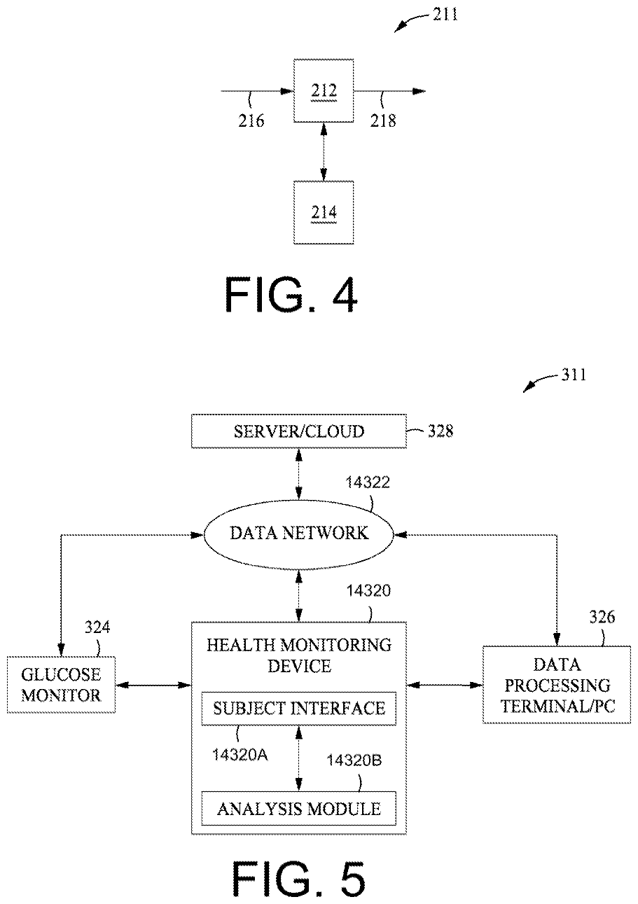

FIG. 4 illustrates an example of a physiological parameter analysis system 211 for providing physiological parameter analysis in accordance with some of the embodiments of the present disclosure. The physiological parameter analysis system 211 includes one or more processors 212 and one or more machine-readable storage media 214. The one or more machine-readable storage media 214 contains a set of instructions for performing a physiological parameter analysis routine, which are executed by the one or more processors 212.

In some embodiments, the instructions include receiving inputs 216 (e.g., one or more RPI values, one or more glucose levels, one or more HbA1c levels, one or more physiological parameters (k_{gly}, k_{age}, and/or K) previously determined, or more other subject-specific parameters, and/or one or more times associated with any of the foregoing), determining outputs 218 (e.g., one or more physiological parameters (k_{gly}, k_{age}, and/or K), an error associated with the one or more physiological parameters, one or more parameters or characteristics for a subject's personalized diabetes management (e.g., cHbA1c, a personalized-target glucose range, an average-target glucose level, a supplement or medication dosage, among other parameters or characteristics), a matched group of participants, and the like), and communicating the outputs 218. In some embodiments, communication of the inputs 216 may be via a user-interface (which may be part of a display), a data network, a server/cloud, another device, a computer, or any combination thereof, for example. In some embodiments, communication of the outputs 218 may be to a display (which may be part of a user-interface), a data network, a server/cloud, another device, a computer, or any combination thereof, for example.

A “machine-readable medium”, as the term is used herein, includes any mechanism that can store information in a form accessible by a machine (a machine may be, for example, a computer, network device, cellular phone, personal digital assistant (PDA), manufacturing tool, any device with one or more processors, and the like). For example, a machine-accessible medium includes recordable/non-recordable media (e.g., read-only memory (ROM), random access memory (RAM), magnetic disk storage media, optical storage media, flash memory devices, and the like).

In some instances, the one or more processors 212 and the one or more machine-readable storage media 214 may be in a single device (e.g., a computer, network device, cellular phone, PDA, an analyte monitor, and the like).

In some embodiments, a physiological parameter analysis system may include other components. FIG. 5 illustrates another example of a physiological parameter analysis system 311 for providing physiological parameter analysis in accordance with some of the embodiments of the present disclosure.

The physiological parameter analysis system 311 includes health monitoring device 14320 with subject interface 14320A and analysis module 14320B. The health monitoring device 14320 is, or may be, operatively coupled to data network 14322. Also provided in physiological parameter analysis system 311 is a glucose monitor 324 (e.g., in vivo and/or in vitro (ex vivo) devices or system) and a data processing terminal/personal computer (PC) 326, each operatively coupled to health monitoring device 14320 and/or data network 14322. Further shown in FIG. 5 is server/cloud 328 operatively coupled to data network 14322 for bi-directional data communication with one or more of health monitoring device 14320, data processing terminal/PC 326 and glucose monitor 324. Physiological parameter analysis system 311 within the scope of the present disclosure can exclude one or more of server/cloud 328, data processing terminal/PC 326 and/or data network 14322.

In certain embodiments, analysis module 14320B is programmed or configured to perform physiological parameter analysis and, optionally, other analyses (e.g., cHbA1c, personalized target glucose range, and others described herein). As illustrated, analysis module 14320B is a portion of the health monitoring device 14320 (e.g., executed by a processor therein). However, the analysis module 14320B may alternatively be associated with one or more of server/cloud 328, glucose monitor 324, and/or data processing terminal/PC 326. For example, one or more of server/cloud 328, glucose monitor 324, and/or data processing terminal/PC 326 may comprise a machine-readable storage medium (or media) with a set of instructions that cause one or more processors to execute the set of instructions corresponding to the analysis module 14320B.

While the health monitoring device 14320, the data processing terminal/PC 326, and the glucose monitor 324 are illustrated as each operatively coupled to the data network 14322 for communication to/from the server/cloud 328, one or more of the health monitoring device 14320, the data processing terminal/PC 326, and the glucose monitor 324 can be programmed or configured to directly communicate with the server/cloud 328, bypassing the data network 14322. The mode of communication between the health monitoring device 14320, the data processing terminal/PC 326, the glucose monitor 324, and the data network 14322 includes one or more wireless communication, wired communication, RF communication, BLUETOOTH® communication, WiFi data communication, radio frequency identification (RFID) enabled communication, ZIGBEE® communication, or any other suitable data communication protocol, and that optionally supports data encryption/decryption, data compression, data decompression and the like.

As described in further detail below, the physiological parameter analysis can be performed by one or more of the health monitoring device 14320, data processing terminal/PC 326, glucose monitor 324, and server/cloud 328, with the resulting analysis output shared in the physiological parameter analysis system 311.

Additionally, while the glucose monitor 324, the health monitoring device 14320, and the data processing terminal/PC 326 are illustrated as each operatively coupled to each other via communication links, they can be modules within one integrated device (e.g., sensor with a processor and communication interface for transmitting/receiving and processing data).

Measuring Glucose and HbA1c Levels

The measurement of the plurality of glucose levels through the various time periods described herein may be done with in vivo and/or in vitro (ex vivo) methods, devices, or systems for measuring at least one analyte, such as glucose, in a bodily fluid such as in blood, interstitial fluid (ISF), subcutaneous fluid, dermal fluid, sweat, tears, saliva, or other biological fluid. In some instances, in vivo and in vitro methods, devices, or systems may be used in combination.

Examples of in vivo methods, devices, or systems measure glucose levels and optionally other analytes in blood or ISF where at least a portion of a sensor and/or sensor control device is, or can be, positioned in a subject's body (e.g., below a skin surface of a subject). Examples of devices include, but are not limited to, continuous analyte monitoring devices and flash analyte monitoring devices. Specific devices or systems are described further herein and can be found in U.S. Pat. No. 6,175,752 and U.S. Patent Application Publication No. 2011/0213225, the entire disclosures of each of which are incorporated herein by reference for all purposes. [0079] In vitro methods, devices, or systems (including those that are entirely non-invasive) include sensors that contact the bodily fluid outside the body for measuring glucose levels. For example, an in vitro system may use a meter device that has a port for receiving an analyte test strip carrying bodily fluid of the subject, which can be analyzed to determine the subject's glucose level in the bodily fluid. Additional devices and systems are described further below.

As described above the frequency and duration of measuring the glucose levels may vary from, on average, about 3 times daily (e.g., about every 8 hours) to about 14,400 times daily (e.g., about every 10 seconds) (or more often) and from about a few days to over about 300 days, respectively.

Once glucose levels are measured, the glucose levels may be used to determine the one or more physiological parameters (k_{gly}, k_{age}, and/or K) and, in some instances, other analyses (e.g., cHbA1c, personalized target glucose range, and others described herein). In some instances, such analyses may be performed with a physiological parameter analysis system. For example, referring back to FIG. 5, in some embodiments, the glucose monitor 324 may comprise a glucose sensor coupled to electronics for (1) processing signals from the glucose sensor and (2) communicating the processed glucose signals to one or more of health monitoring device 14320, server/cloud 328, and data processing terminal/PC 326.

The measurement of one or more HbA1c levels at the various times described herein may be according to any suitable method. Typically, HbA1c levels are measured in a laboratory using a blood sample from a subject. Examples of laboratory tests include, but are not limited to, a chromatography-based assay, an antibody-based immunoassay, and an enzyme-based immunoassay. HbA1c levels may also be measured using electrochemical biosensors.

The frequency of HbA1c level measurements may vary from, on average, monthly to annually (or less often if the average glucose level of the subject is stable).

Calculated HbA1c (cHbA1c)

Referring back to FIG. 5, in some embodiments, HbA1c levels may be measured with a laboratory test where the results are input to the server/cloud 328, the subject interface 14320A, and/or a display from the testing entity, a medical professional, the subject, or other user. Then, the HbA1c levels may be received by the one or more of health monitoring device 14320, server/cloud 328, and data processing terminal/PC 326 for analysis by one or more methods described herein.

After one or more physiological parameters (k_{gly}, k_{age}, and/or K) are calculated, a plurality of glucose measurements may be taken for a following time period and used for calculating HbA1c during and/or at the end of the following time period. For example, referring back to FIG. 3, one or more physiological parameters (k_{gly}, k_{age} and/or K) may be calculated at time point 101 based on one or more measured RPI values 110a, 110b, measurements of the plurality of glucose levels 12104a over time period 106, a measured HbA1c level 12102b at the end of time period 106, and optionally a measured HbA1c level 12102a at the beginning of time period 106. Then, for a subsequent time period 108, a plurality of glucose levels 12104b may be measured. Then, during and/or at the end of the time period 108, Equation 8 can be used to determine a cHbA1c value (HbA1c_{z} of Equation 8) where HbA1cₒ is the measured HbA1c level 12102b at the end of time period 106 (which is the beginning of time period 108), [G,] are the glucose levels or averaged glucose levels during time period 108 (or the portion of time period 108 where cHbA1c is determined during the time period 108), and the provided one or more physiological parameters (k_{gly}, k_{age}, and/or K) corresponding to time point 101 are used.

A subject's cHbA1c may be determined for several successive time periods based on the one or more physiological parameters (k_{gly}, k_{age}, and/or K) determined with the most recently measured HbA1c level, the most recently measured RPI value(s), and the intervening measurements of glucose levels. The RPI value may be measured periodically (e.g., every 6 months to a year) to recalculate k_{age}. The most recent RPI value or an average of two or more RPI values can be used in the calculation. The HbA1c may be measured periodically (e.g., every 6 months to a year) to recalculate the one or more physiological parameters. The time between remeasuring the RPI value and the measured HbA1c may depend on (1) the consistency of the measurements of glucose levels, (2) the frequency of the measurements of glucose levels, (3) a subject's and corresponding family's diabetic history, (4) the length of time the subject has been diagnosed with diabetes, (5) changes to a subject's personalized diabetes management (e.g., changes in medications/dosages, changes in diet, changes in exercise, and the like), (6) the presence of a disease or disorder that effects kₘₐₜ (e.g., anemia, a bone marrow disease, a genetic condition, an immune system disorder, and combinations thereof). For example, a subject with consistent measurements of glucose levels (e.g., a [G] with less than 5% variation) and frequent measurements of glucose levels (e.g., continuous glucose monitoring) may measure HbA1c levels less frequently than a subject who recently (e.g., within the last 6 months) changed the dosage of a glycation medication, even with consistent and frequent measurements of glucose levels.

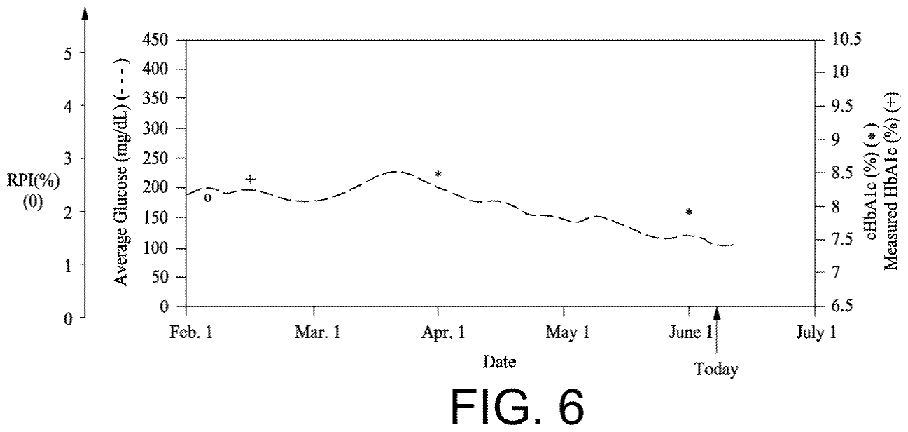

FIG. 6, with reference to FIG. 4, illustrates an example of a cHbA1c report that may be generated as an output 218 by a physiological parameter analysis system 211 of the present disclosure. The illustrated example report includes a plot of average glucose level over time. Also included on the report are the most recently measured RPI value (open circle), the most recently measured HbA1c level (cross), and cHbA1c levels (asterisks) calculated by the physiological parameter analysis system 211. While the most recently measured RPI value and the most recently measured HbA1c level are illustrated as being measured on different days, the two measurements can be done in the same visit to a health care provider.

Two cHbA1c levels are illustrated, but one or more cHbA1c levels may be displayed on the report, including a line that continuously tracks cHbA1c. Alternatively, the output 218 of the physiological parameter analysis system 211 may include a single number for a current or most recently calculated cHbA1c, a table corresponding to the data of FIG. 6, or any other report that provides a subject, healthcare provider, or the like with at least one cHbA1c level.

In some instances, the cHbA1c may be compared to a previous cHbA1c and/or a previous measured HbA1c level to monitor the efficacy of a subject's personalized diabetes management. For example, if a diet and/or exercise plan is being implemented as part of a subject's personalized diabetes management, with all other factors (e.g., medication and other diseases) equal, then changes in the cHbA1c compared to the previous cHbA1c and/or the previous measured HbA1c level may indicate if the diet and/or exercise plan is effective, ineffective, or a gradation therebetween.

In some instances, the cHbA1c may be compared to a previous cHbA1c and/or a previous measured HbA1c level to determine if another HbA1c measurement should be taken. For example, in the absence of significant glucose profile change, if the cHbA1c changes by 0.5 percentage units or more (e.g., changes from 7.0% to 6.5% or from 7.5% to 6.8%) as compared to the previous cHbA1c and/or the previous measured HbA1c level, another measured HbA1c level may be tested.

In some instances, a comparison of the cHbA1c to a previous cHbA1c and/or a previous measured HbA1c level may indicate if an abnormal or diseased physiological condition is present. For example, if a subject has maintained a cHbA1c and/or measured HbA1c level for an extended period of time, then if a change in cHbA1c is identified with no other obvious causes, the subject may have a new abnormal or diseased physiological condition. Indications of what that new abnormal or diseased physiological condition may be gleaned from the one or more physiological parameters (k_{gly}, k_{age}, and/or K). Details of abnormal or diseased physiological conditions relative to the one or more physiological parameters are discussed further herein.

Personalized-Target Glucose Range

Typically, the glucose levels in subjects with diabetes are preferably maintained between 54 mg/dL and 180 mg/dl. However, the kinetic model described herein (see Equation 6) illustrates that intracellular glucose levels are dependent on physiological parameters k_{gly}, k_{age}, and K. Therefore, a measured glucose level may not correspond to the actual physiological conditions in a subject. For example, a subject with a higher than normal K may glycate glucose more readily. Therefore, a 180 mg/dl measured glucose level may be too high for the subject and, in the long run, potentially worsen the effects of the subject's diabetes. In another example, a subject with a lower than normal k_{gly} may glycate glucose to a lesser degree. Accordingly, at a 54 mg/dL glucose level, the subject's intracellular glucose level may be much lower making the subject feel weak and, in the long term, lead to the subject being hypoglycemic.

Using the accepted normal lower glucose limit (LGL) and the accepted normal HbA1c upper limit (AU), equations for a personalized lower glucose limit (GL) (Equation 13) and a personalized upper glucose limit (GU) (Equation 14) can be derived from Equation 6.

GL=(LGL*¾J)//c¾? Equation 13

where k^{re}ₜ\hat{ }is the k_{gly} for a normal person and kjffi is the subject's k_{gly}.

GU=AU/(K(1−AU)) Equation 14

Equation 13 is based on k_{gly} because the lower limit of a glucose range is based on an equivalent intracellular glucose level. Equation 14 is based on K because the upper limit of a glucose range is based on an equivalent extracellular glucose level (e.g., the accepted normal HbA1c upper limit).

The currently accepted values for the foregoing are LGL=54 mg/dL, [symbol] =6.2*10⁻⁶ dL*mg⁻¹*day⁻¹, and AU=0.08 (i.e., 8%). Using the currently accepted values Equations 15 and 16 can be derived.

GL=3.35*10⁻⁴ day⁻¹/k Equation 15

GU=0.087/K Equation 16

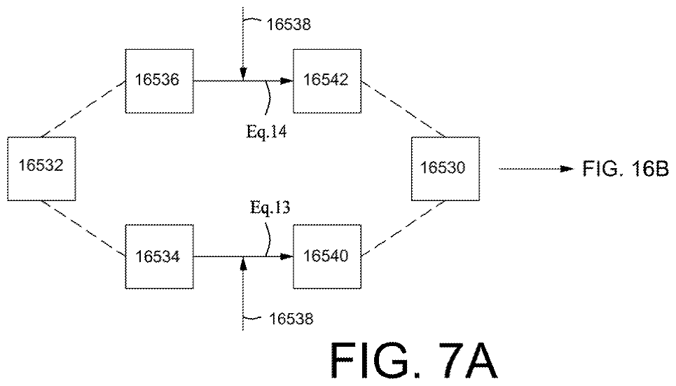

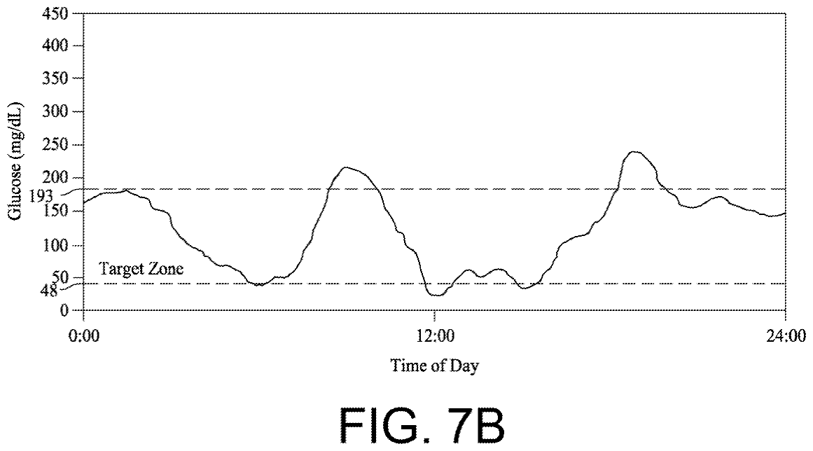

FIG. 7A illustrates an example of a method of determining a personalized-target glucose range 16530. A desired intracellular glucose range 16532 (e.g., the currently accepted glucose range) having a lower limit 16534 and an upper limit 16536 can be personalized using one or more determined physiological parameters (k_{gly}, k_{age} and/or K) 16538 using Equation 13 and Equation 14, respectively. This results in a personalized lower glucose limit (GL) 16540 (Equation 13±7%) and a personalized upper glucose limit (GU) 16542 (Equation 14±7%) that define the personalized-target glucose range 16530. After one or more physiological parameters (k_{gly}, k_{age}, and/or K) are calculated, a personalized-target glucose range may be determined where the lower glucose limit may be altered according to Equation 13 (or Equation 15)±7% and/or the upper glucose limit may be altered according to Equation 14 (or Equation 16)±7%. The ±7% relative to each of the foregoing calculated values allows for a different value that is substantially close to the calculated value to be used, so that the personalized nature of the personalized-target glucose range 16530 is maintained. Alternatively, the ±7% can be ±10%, ±5%, or ±3%.

For example, a subject with a K of 4.5*10⁻⁴ dL/mg and a k_{gly} of 7.0*10⁶ dL*mg⁻¹*day¹ may have a personalized-target glucose range of about 48±3.4 mg/dl to about 193±13.5 mg/dl. Therefore, the subject may have a wider range of acceptable glucose levels than the currently practiced glucose range.

FIG. 7B, with reference to FIG. 4, illustrates an example of a personalized-target glucose range report that may be generated as an output 218 by a physiological parameter analysis system 211 of the present disclosure. The illustrated example report includes a plot of glucose level over a day relative to the foregoing personalized-target glucose range (area between the dashed lines). Alternatively, other reports may include, but are not limited to, an ambulatory glucose profile (AGP) plot, a numeric display of the personalized-target glucose range with the most recent glucose level measurement, and the like, and any combination thereof.

In another example, a subject with a K of 6.5*10⁻⁴ dL/mg and a k_{gly} of 6.0*10⁶ dL*mg⁻¹*day¹ may have a personalized-target glucose range of about 56±3.5 mg/dL to about 134±10 mg/dL. With the much-reduced upper glucose level limit, the subject's personalized diabetes management may include more frequent glucose level measurements and/or medications to stay substantially within the personalized-target glucose range.

In yet another example, a subject with a K of 5.0*10⁻⁴ dL/mg and a k_{gly} of 5.0*10⁻⁶ dL*mg⁻¹*day may have a personalized-target glucose range of about 67±4.5 mg/dL to about 174±12 mg/dL. This subject is more sensitive to lower glucose levels and may feel weak, hungry, dizzy, etc. more often if the currently practiced glucose range (54 mg/dL and 180 mg/dL) were used.

While the foregoing examples all include a personalized glucose lower limit and a personalized glucose upper limit, a personalized-target glucose range may alternatively include only the personalized glucose lower limit or the personalized glucose upper limit and use the currently practiced glucose lower or upper limit as the other value in the personalized-target glucose range.

The personalized-target glucose range may be determined and/or implemented in a physiological parameter analysis system. For example, a set of instructions or program associated with a glucose monitor and/or health monitoring device that determines a therapy (e.g., an insulin dosage) may use a personalized-target glucose range in such analysis. In some instances, a display or subject interface with display may display the personalized-target glucose range.

The personalized-target glucose range may be updated over time as one or more physiological parameters are recalculated.

Personalized-Target Average Glucose

In some instances, a subject's personalized diabetes management may include having an HbA1c value target for a future time point. For example, referring to FIG. 3, a subject may have a measured RPI value 110b and a measured HbA1c value 12102b for time point 101 and a plurality of glucose level measurements prior thereto over time period 106. The subject's personalized diabetes management may include a target HbA1c value (AT) for time point 12103 that would correlate to improved health for the subject. Equation 17 can be used to calculate a personalized-target average glucose level (GT) for the next time period 108 and be based on the target HbA1c value (AT) and the subject's K calculated at time point 101.

GT=AT/(K(1−AT)) Equation 17

In some embodiments, a physiological parameter analysis system may determine an average glucose level for the subject during time period 108 and, in some instances, display the average glucose level and/or the target average glucose level. The subject may use the current average glucose level and the target average glucose level to self-monitor their progress over time period 108. In some instances, the current average glucose level may be transmitted (periodically or regularly) to a health care provider using a physiological parameter analysis system for monitoring and/or analysis.

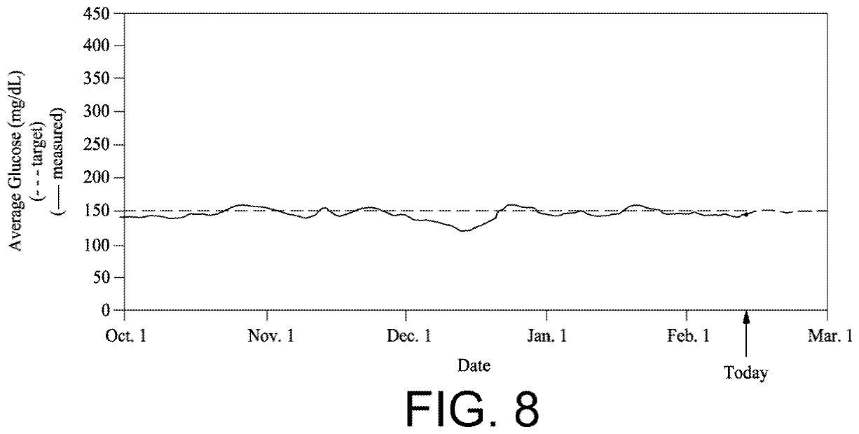

FIG. 8, with reference to FIG. 4, illustrates an example of a personalized-target average glucose report that may be generated as an output 218 by a physiological parameter analysis system 211 of the present disclosure. The illustrated example report includes a plot of a subject's average glucose (solid line) over time and the personalized-target average glucose (illustrated at 150 mg/dL, dashed line). Alternatively, other reports may include, but are not limited to, a numeric display of the personalized-target average glucose with the subject's average glucose level over a given time frame (e.g., the last 12 hours), and the like, and any combination thereof.

The personalized-target average glucose may be updated over time as one or more physiological parameters are recalculated.

### EXAMPLES

Data from 148 type 2 and 139 type 1 subjects enrolled in two previous clinical studies having six months of continuous glucose monitoring were analyzed. Only 90 subjects had sufficient data to meet the kinetic model assumptions described above having data with no continuous glucose data gap 12 hours or longer. Study participants had three HbA1c measurements, on days 1, 100 (±5 days), and 200 (±5 days), as well as frequent subcutaneous glucose monitoring throughout the analysis time period, which allowed for analysis of two independent data sections (days 1-100 and days 101-200) per participant.

The first data section (days 1-100) was used to numerically estimate individual k_{gly} and k_{age}, which allows prospective calculation of ending cHbA1c of the second data section (days 101-200). This ending cHbA1c can be compared with the observed ending HbA1c to validate the kinetic model described herein. For comparison, an estimated HbA1c for the second data section was calculated based on (1) 14-day mean and (2) 14-day weighted average glucose converted by the accepted regression model from the Ale-Derived Average Glucose (ADAG) study, which both assume k_{gly} is a constant, which as discussed previously is the currently accepted method of relating HbA1c to glucose measurements.

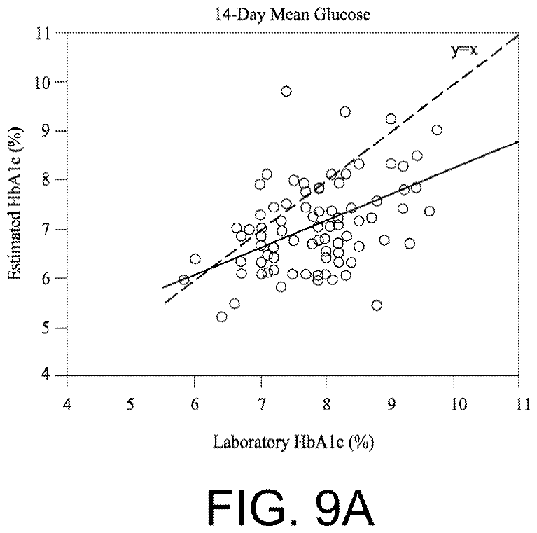

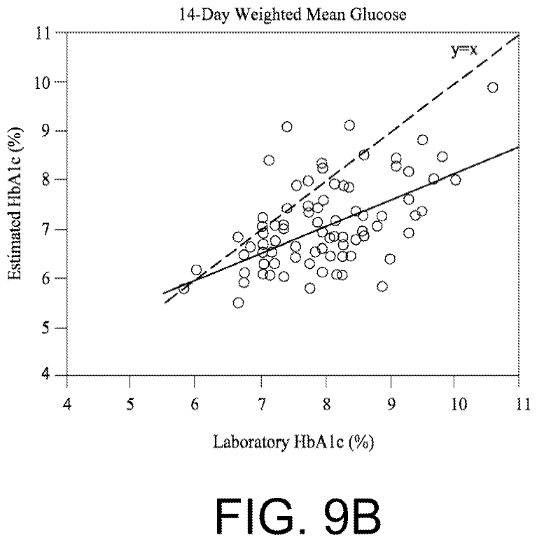

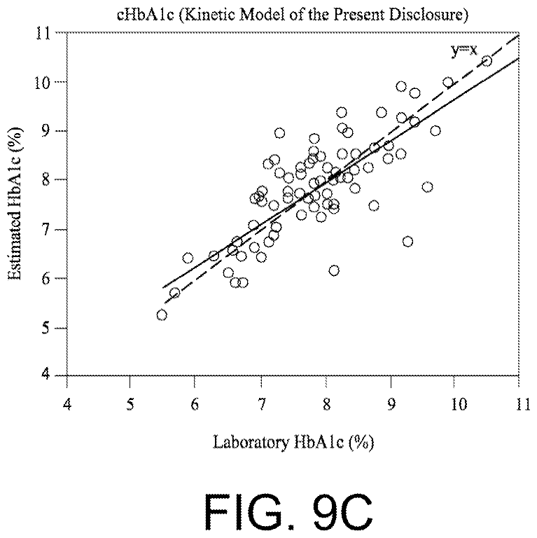

FIGS. 9A-C illustrate a comparison between the laboratory HbA1c levels at day 200 (±5 days) relative to the estimated HbA1c values, where the eHbA1c values in the 18A plot are calculated using the 14-day mean model, the eHbA1c values in the 18B plot are calculated using the 14-day weighted average model, and the cHbA1c values in the 18C plot are calculated using the kinetic model described herein (Equation 8). The solid line in all graphs illustrates the linear regression of the comparative HbA1c values for the corresponding models. The dashed line is a one-to-one line, where the closer the solid line linear regression is thereto, the better the model. Clearly, the kinetic model described herein models the data better, which illustrates that k_{age} and k_{gly} are individualized, which is a novel way to approach correlating HbA1c to glucose measurements.

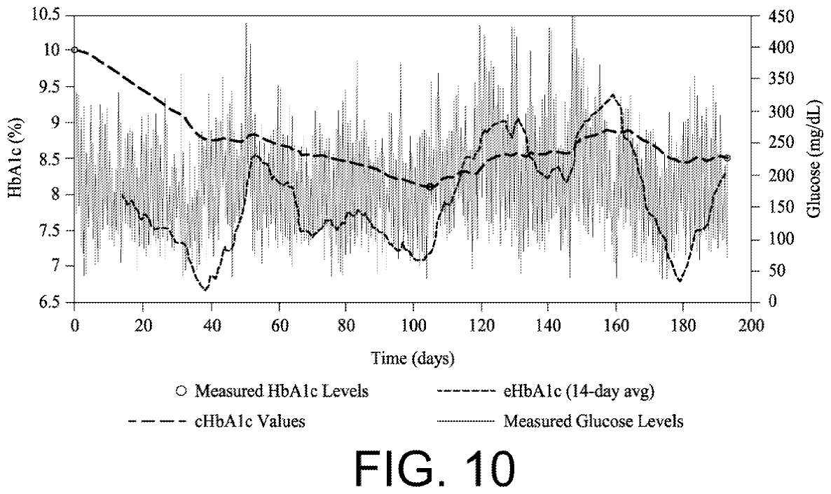

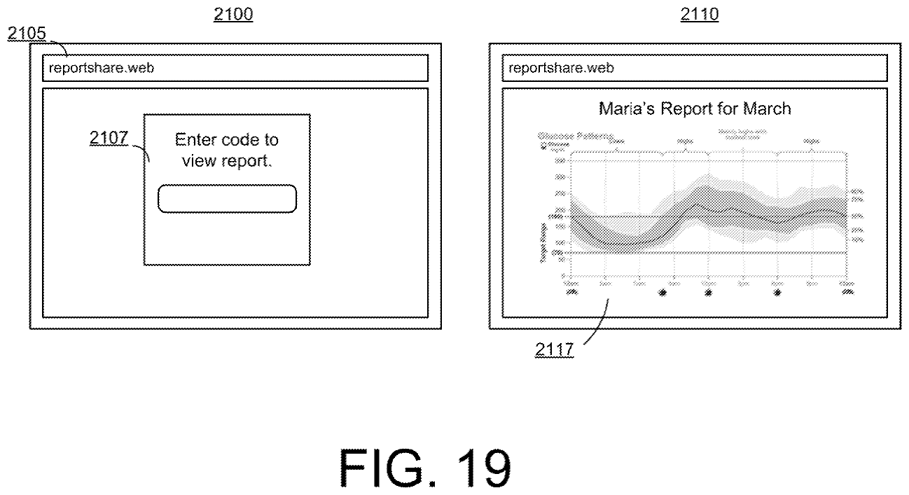

FIG. 10 illustrates an example study subject's data with the measured glucose levels (solid line), laboratory HbA1c readings (open circles), cHbA1c model values (long dashed line), and 14-day eHbA1c model values (dotted line). The cHbA1c model values in FIG. 19 were calculated using the physiological parameters (k_{age} and k_{gly}). The physiological parameters were calculated based on the first two laboratory HbA1c readings and glucose levels measured between the first two laboratory HbA1c readings. The 14-day eHbA1c values are glucose level 14-day running averages during the study.

The FIG. 10 example shows the dynamic nature of the glucose-to-cHbA1c and glucose-to-eHbA1c relationships. Additional examples were determined for type 1 and type 2 diabetes study participants across a range of prediction deviations: 25th, 50th and 75th percentiles for the cHbA1c method. In these examples, the disagreement between the cHbA1c from the 14-day average glucose indicates the exaggerated amplitude of variation inherent in the simple 14-day method.

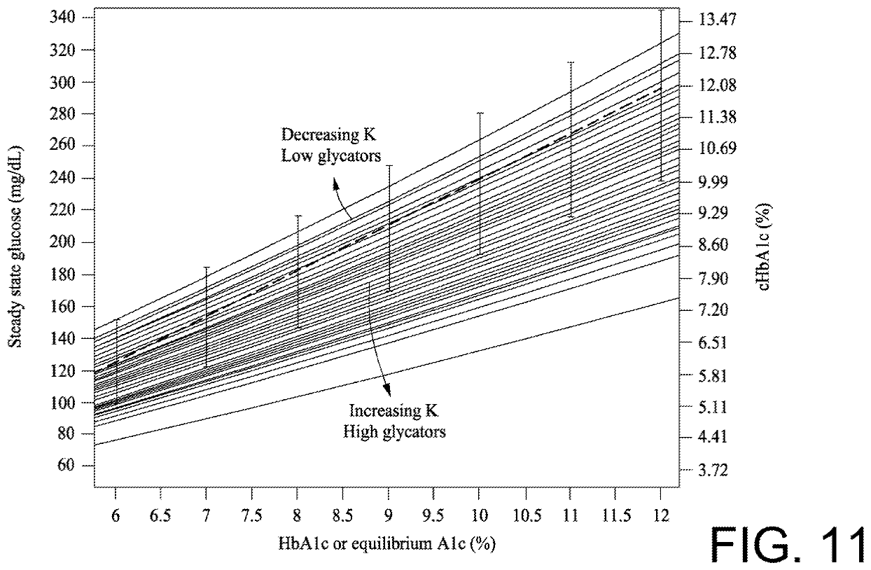

FIG. 11 illustrates the relationship between steady glucose and equilibrium HbA1c (1) as determined using the standard conversion of HbA1c to estimated average glucose (dashed line with error bars) and (2) as measured for the 90 participants (solid lines). These individual curves (solid lines) represent the agreement of average glucose with laboratory measure HbA1c under the condition of their average glucose level being stable for days-to-weeks. The model suggests that the relationship of glucose-to-HbA1c is not constant, with larger changes in glucose needed to achieve the same change in HbA1c as levels of the latter marker increase. Contrary to prior assessments of the glycation index, the kinetic model of the present disclosure suggests that an individual's glycation index will not be constant across all levels of HbA1c. Unlike eHbA1c, a key advantage of cHbA1c is its ability to account for individual variation in glycation. Individuals with lower K are “low glycators”, and have higher average glucose levels for a given HbA1c level, with the reverse being true for those with high K values.

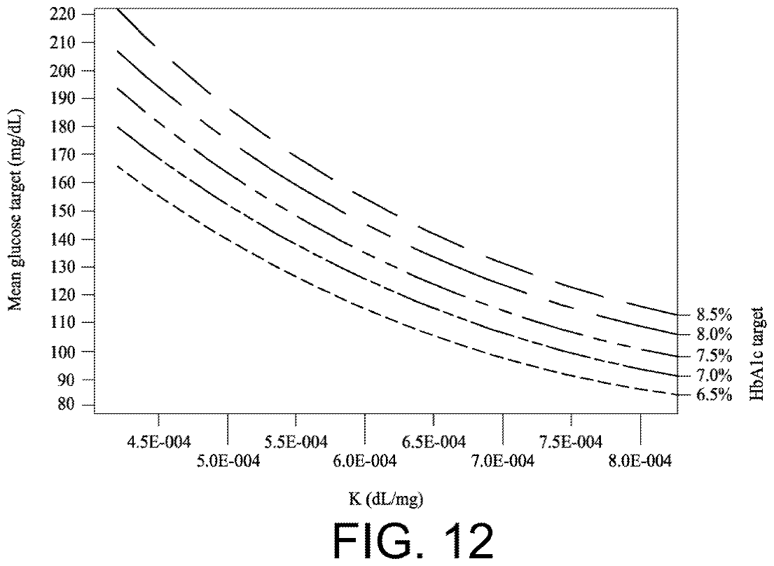

Using the kinetic model of the present disclosure, a relationship between K (dL/mg) and mean glucose level target (mg/dL) is illustrated in FIG. 12 plotted for varying HbA1c target values. That is, if a subject is targeting a specific HbA1c value (e.g., for a subsequent HbA1c measurement or cHbA1c estimation) and has a known K value (e.g., based on a plurality of measured glucose levels and at least one measured HbA1c), a mean glucose target can be derived and/or identified for the subject over the time period in which the subject is targeting the HbA1c value.

Additional details of methods, devices, and systems for determining physiological parameters related to the kinetics of red blood cell glycation, elimination, and generation within the body of a subject are set forth in U.S. Patent Publication No. 2018/0235524 to Dunn et al., International Publication No. WO2020/086934 to Xu, International Publication No. WO2021/108419 to Xu, International Publication No. WO2021/108431 to Xu, U.S. Provisional Patent Application No. 62/939,970, U.S. Provisional Patent Application No. 63/015,044, U.S. Provisional Patent Application No. 63/081,599, U.S. Provisional Patent Application No. 62/939,956, each of which is incorporated by reference in its entirety herein. Such physiological parameters can be used, for example, to calculate personalized glucose metrics or personalized analyte measurements: a more reliable calculated HbA1c (cHbA1c) or glucose-derived A1c (GD-A1c), adjusted HbA1c (aHbA1c or personalized A1c), adjusted cA1c (or cHbA1c adjusted by K_{age}), and/or a personalized target glucose range, among other things, for subject-personalized diagnoses, treatments, and/or monitoring protocols.

For purpose of illustration, not limitation, the processor in the reader device is configured to run the models described herein to calculate the physiological parameters and personalized glucose metrics. As embodied herein, the laboratory A1c measurement required to calculate the physiological parameters and the personalized glucose metrics can be received by the reader device, for example, not limitation, by using a camera (for example, not limitation, such as one built into the reader device) to scan a QR code which includes the relevant laboratory A1c data. As embodied herein, the laboratory A1c measurement can be received or retrieved by the reader device from a cloud-based database. As embodied herein, a home testing kit can be used to measure HbA1c in a blood sample and can be entered into the reader device by the user, instead of a laboratory A1c measurement.

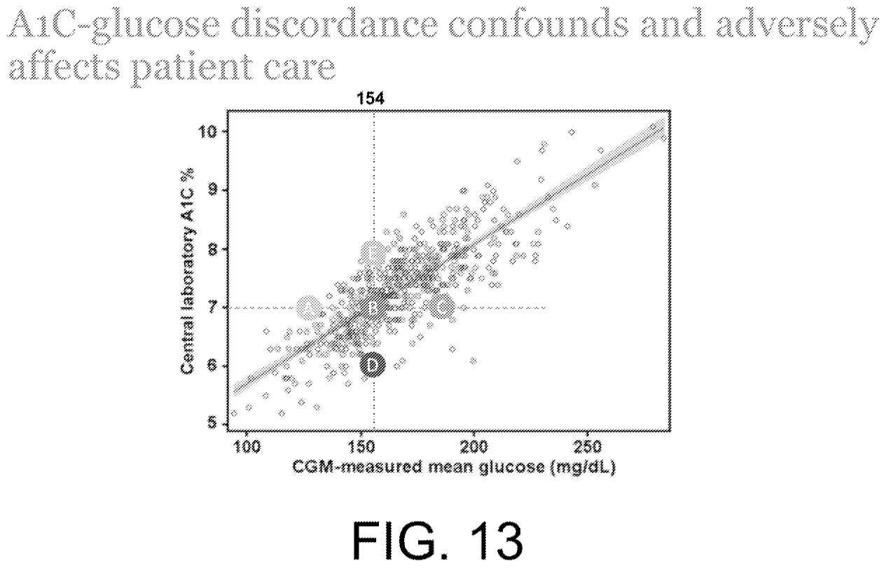

A1c-glucose discordance confounds and adversely affects subject care. For example, as shown in Table 1 below, subjects A, B, and C have the same laboratory measured A1c levels but different mean glucose levels (125 mg/dL, 154 mg/dL, and 188 mg/dL, respectively). Similarly, subjects B, D, and E have same mean glucose level of 154 mg/dL, but different laboratory measured A1c (7.0%, 6.0%, and 8.0%, respectively). This information is represented graphically in FIG. 13.

TABLE 1

| | Mean Glucose | |
|---|---|---|
| Subject | (mg/dL) | Lab A1c (%) |
| A | 125 | 7.0 |
| B | 154 | 7.0 |
| C | 188 | 7.0 |
| D | 154 | 6.0 |
| E | 154 | 8.0 |

Models described herein allow quantitative removal of red blood cell artifacts, thereby improving hyperglycemia risk assessment. For example, for illustration not limitation, consider the subjects A-E with the following characteristics:

TABLE 2

| | RBC Lifespan | | Personalized |
|---|---|---|---|
| Subject | (days) | Lab A1c (%) | A1c (%) |
| A | 123 | 7.0 | 6.0 |
| B | 87 | 7.0 | 8.4 |
| C | 110 | 7.0 | 6.7 |
| D | 89 | 6.0 | 7.1 |
| E | 121 | 8.0 | 6.9 |

As can be seen in Table 2, subjects A, B, and C have different RBC lifespan (or as measured in days (123, 87, and 110, respectively) but the same laboratory measured A1c of 7.0%. Based on the different RBC lifespan, subject A, B, and C's personalized A1c or adjusted A1c, as measured by the models disclosed above, is 6, 8.4, and 6.7, respectively. Since the laboratory measured A1c for the three subjects is the same, their respective medical providers may view all three as diabetic and prescribe the same treatment regimen based on these values. However, because of their differing RBC lifespan, their glycemic control is in fact very different, as demonstrated by their starkly different personalized A1c. Indeed, based on their respective personalized A1c, subject A is pre-diabetic (based on personalized A1c of 6.0), subject B is clearly diabetic (based on personalized A1c of 8.4), and subject C is also diabetic (based on personalized A1c of 6.7). Accordingly, subjects A, B, and C in fact may need different treatment regimens. Similarly, although subject D may be viewed as pre-diabetic based on laboratory A1c of 6.0, they would be considered diabetic based on personalized A1c of 7.1. Further, subject E would be considered diabetic based on a laboratory A1c of 8.0, but would be considered pre-diabetic based on personalized A1c of 6.9.

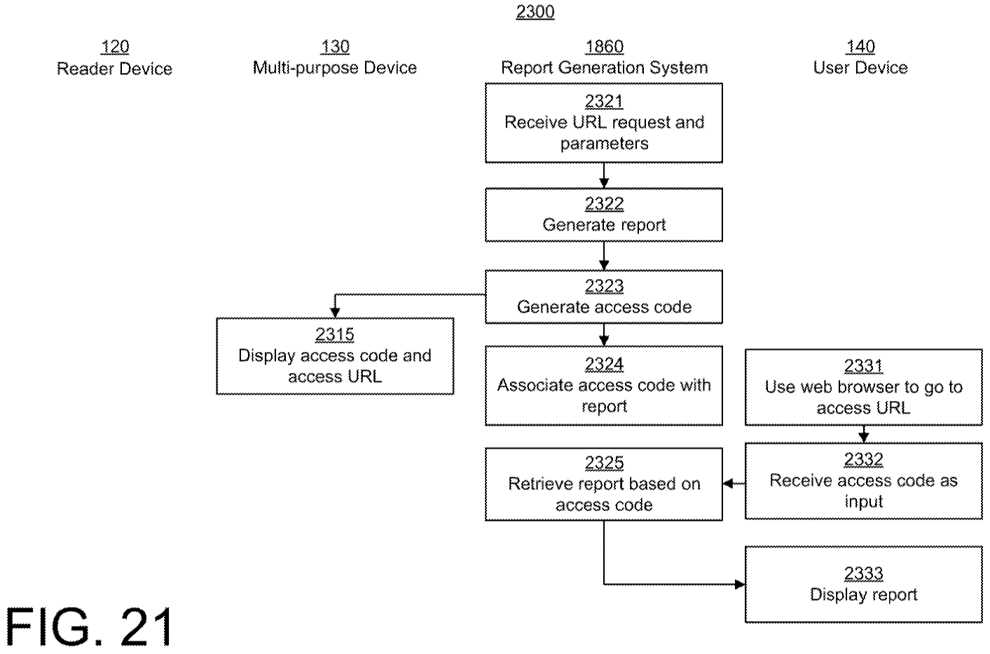

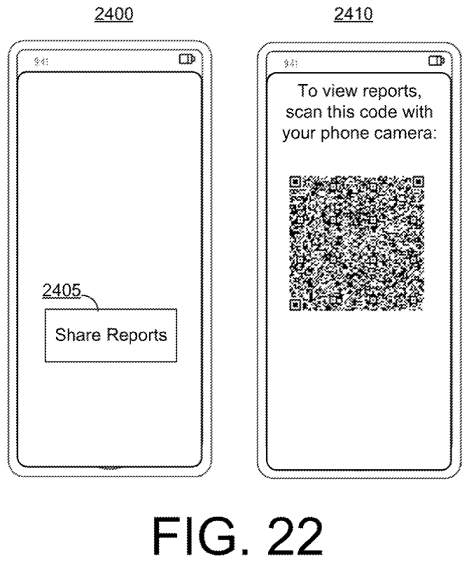

FIGS. 21-27 provide exemplary case studies illustrating the application of the models as described herein. For example, as can be seen in FIG. 21, exemplary subjects J17, J33, and J5 have a measured mean glucose of 148 mg/dL, 149 mg/dL, and 153 mg/dL, respectively, and laboratory A1c of 7.7%, 6.8%, and 8.1%. However, their personalized mean glucose and personalized A1c, as determined using the models described herein, differ significantly. Specifically, J17, J33, and J5 have a personalized mean glucose of 141 mg/dL, 250 mg/dL, and 130 mg/dL, respectively, and laboratory A1c of 6.7%, 9.5%, and 6.8%. Notably, J33's lab measured glucose metrics are starkly different than their personalized glucose metrics. FIG. 21 provides a graphical representation of these metrics. These and other metrics shown in FIG. 21 can also be seen in FIGS. 22-27.

### Example Embodiments of Data Integration with Electronic Health/Medical Records

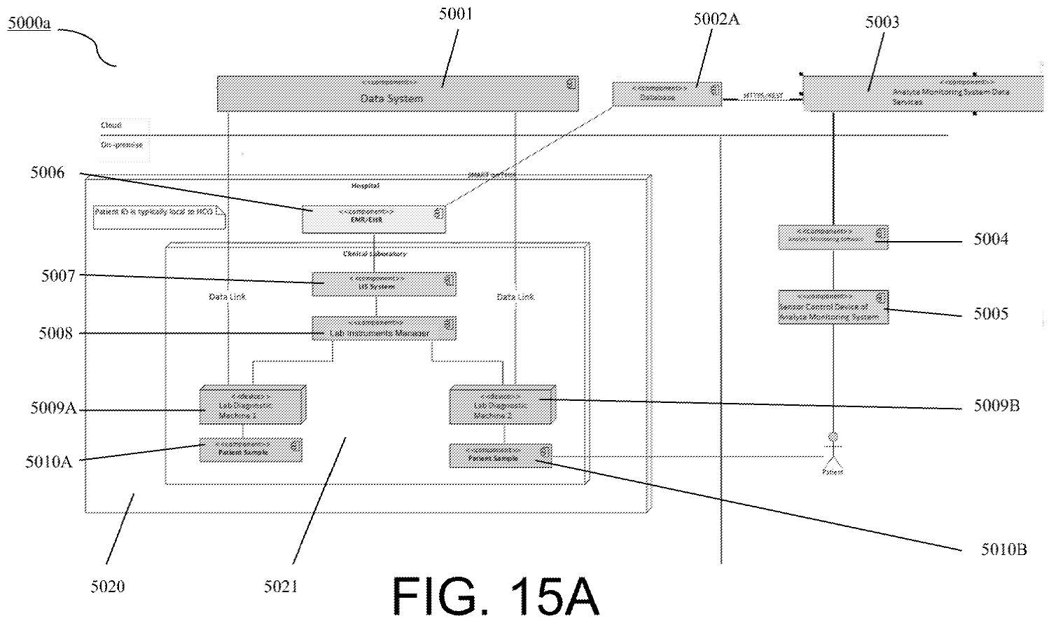

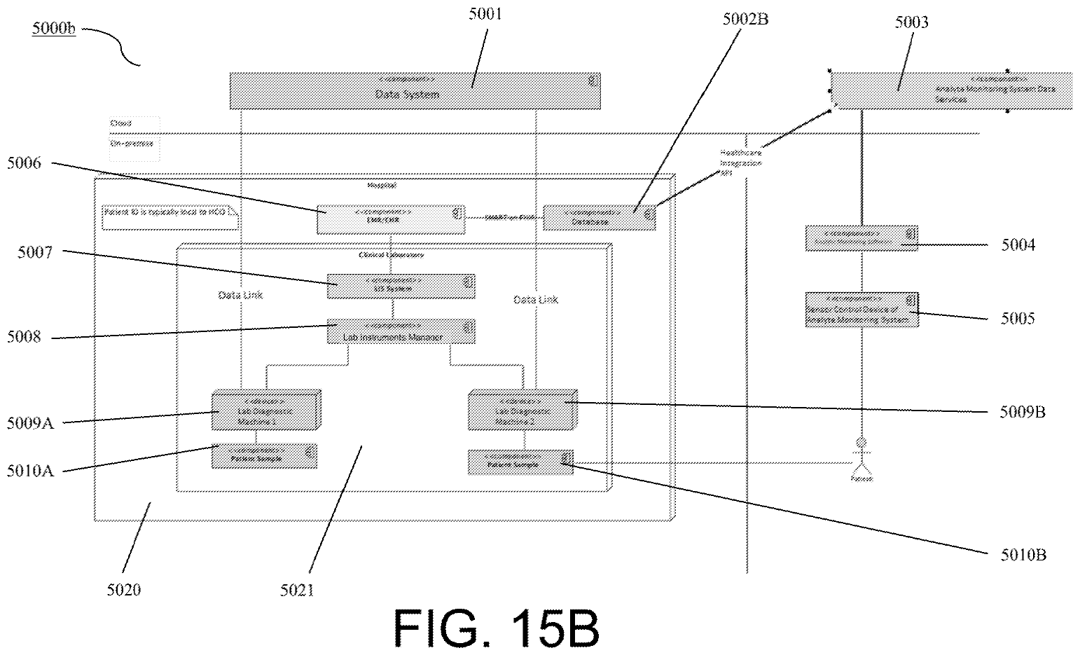

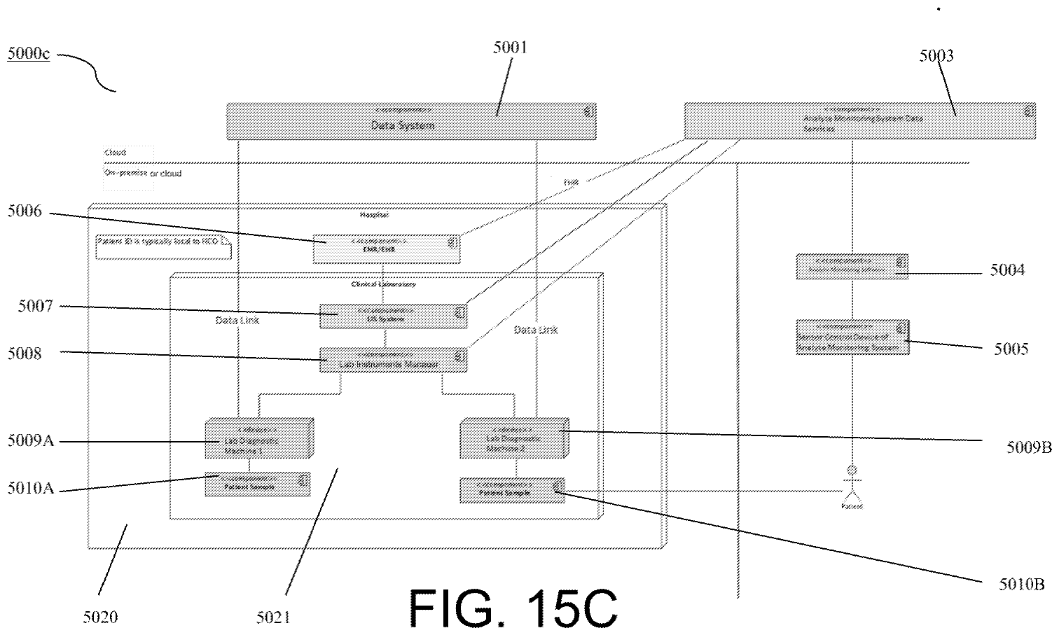

Described herein are example embodiments of systems and methods for bi-directional communication of patient data (for example, without limitation, laboratory HbA1c, glucose concentration data, continuous glucose monitoring data, physiological parameters, etc.). According to one aspect of the embodiments, as shown in FIG. 15A, system 5000a for bi-directional communication of patient data can include a database (e.g., hospital or health care organization (HCO)) 5020, another database 5002A, and data services 5003 (e.g., in some embodiments, analyte monitoring system data services).

Analyte monitoring system data services 5003 can be a trusted computer system 180, as described above, and can include a cloud-based server or network including software to provide services including, for example and without limitation, authentication and user profile management, secured data transmission and storage, and analyte data report generation. Analyte monitoring software 5004 can be a user interface application (e.g., software) such as those described above, and can be a reader device 120. According to embodiments, data services 5003 can store analyte measurements generated and transmitted by a plurality of reader devices and sensor control devices in communication with data service 5003. Additional details regarding receipt of analyte measurements by data services 5003 are disclosed below. According to embodiments, data service 5003 maintains a record of stored analyte measurements by associating them with appropriate user ID. For example, not limitation, user ID can include email address, date of birth, first name, last name, address, geographic location of the patient, social security number, phone number, etc. or any combination thereof.

According to embodiments, as can be seen in FIG. 15A, hospital 5020 can include an electronic medical/health records component 5006 in communication with clinical laboratory 5021. Clinical laboratory 5021 can include a laboratory information system or LIS system 5007, lab instruments manager 5008, and one or more lab diagnostic machines 5009A, B (two shown). In certain embodiments, LIS system 5007 can be contained within EMR/EHR 5006 (for example, without limitation, as a module). Additionally or alternatively, LIS system 5007 can include the functionality of lab instruments manager 5008. According to embodiments, one or more lab diagnostic machines 5009A, B can communicate with data system 5001 using a data link, which can include a wired or wireless technique. One or more lab diagnostic machines 5009A, B are configured to receive patient sample 5010A, B, respectively, and perform laboratory analysis on the received samples. In operation, electronic medical/health records component 5006 generates and sends an order to the LIS system 5007 for performance of laboratory analysis for a particular patient. The LIS system 5007 sends the order to lab instruments manager 5008, which sends the order to the appropriate lab diagnostic machines 5009A, B. Once a patient sample is received by lab diagnostic machines 5009A, B, laboratory analysis is performed and the results from the laboratory analysis are transmitted to the lab instruments manager 5008, which transmits the results to LIS system 5007, which in turn transmits the results to the electronic medical/health records 5006.

According to embodiments, patient sample 5010A, B can include, without limitation, any other biological fluid or sample known to a person of ordinary skill in the art, such as blood, urine, etc. According to embodiments, laboratory analysis can include, without limitation, analyzing how well organs such as kidneys, liver, thyroid, heart, etc. are working. For example, not limitation, results of the laboratory analysis can include a patient's HbA1c, cholesterol, lipid panel, CBC, any other assay of medical relevance, etc.

According to embodiments, the electronics medical/health records (EMR) 5006 generates a patient ID corresponding to a personal identification of a patient. Thereafter, the patient ID is transmitted in conjunction with the generated test order to the LIS system 5007, which generates a sample ID corresponding to the test order. As such, the sample ID remains specific to the LIS system 5007 and sample ID alone is associated with the order within clinical laboratory 5021 environment. Once laboratory analysis is complete and results from the laboratory analysis are transmitted to the LIS system 5007, LIS system 5007 pairs the sample ID associated with the results to the appropriate patient ID. Accordingly, a record of the results from the laboratory analysis can be associated with a patient ID. For example, not limitation, patient ID can include email address, date of birth, first name, last name, address, geographic location of the patient, social security number, phone number, etc. or any combination thereof. Patient ID represents a unique identification metric for each patient at the HCO. In some embodiments, patient ID is specific to each hospital. Therefore, the same patient may not have the same patient ID across different HCOs. In other words, the same patient can have different patient IDs at different HCOs.

As can be seen in FIG. 15A, database 5002A can be a database maintained on a remote cloud server in communication with data services 5003 and electronics medical/health records 5006 and can include one or more processors (not shown). Database 5002A can receive a record of results from the laboratory analysis along with the associated patient ID from EMR 5006 and a record of analyte measurements along with the associated user ID from data services 5003. Thereafter, one or more processors can pair the results from the laboratory analysis with the analyte measurements based upon a shared data item contained in the records. For example, not limitation, if a patient ID matches with a user ID, then laboratory results associated with the patient ID can be paired with the analyte measurements associated with the user ID. For example, not limitation, shared data item can include email address, date of birth, first name, last name, address, geographic location of the patient, social security number, phone number, etc. or any combination thereof. One or more processors can further receive a request to read, write, edit, or delete a resource data in the first or second database, wherein the request is formatted according to a well-defined protocol, for example, not limitation, Fast Healthcare Interoperability Resources (FHIR) standard and FHIR extensions embodying a healthcare provider directory (HPD) standard, or HL7.

According to embodiments, one or more processors of database 5002A can perform a calculation based on the laboratory results and the analyte measurements. Alternatively or additionally, one or more processors of analyte monitoring system database services 5003 or analyte monitoring software 5004 can perform a calculation based on the laboratory results and the analyte measurements. For example, not limitation, where the laboratory results include HbA1c and the analyte measurements include glucose measurements, the one or more processors can perform a calculation of a glucose derived A1c or a kinetic model for determining A1c. Additional details of calculation of a glucose-derived A1c or a kinetic model for determining A1c are set forth in U.S. Patent Publication No. 2018/0235524 to Dunn et al., International Publication No. WO 2020/086934 to Xu, U.S. Provisional Patent Application No. 62/939,970, U.S. Provisional Patent Application No. 62/939,956, U.S. Provisional Patent Application No. 63/015,044, and U.S. Provisional Patent Application No. 63/081,599, each of which is incorporated by reference in its entirety herein.

According to embodiments, database 5002A can communicate with electronics medical/health records 5006 according to a well-defined protocol, for example, not limitation, Fast Healthcare Interoperability Resources (FHIR) standard, such as those disclosed in U.S. Patent Publication No. 2017/0270532, which is incorporated herein in its entirety. In some embodiments, database 5002A can communicate with electronics medical/health records 5006 using SMART on FHIR. According to embodiments, database 5002A can communicate with data services 5003 according to a well-defined protocol, for example, not limitation, Hypertext Transfer Protocol Secure (HTTPS) or Representational State Transfer (REST) protocol.

According to embodiments, as can be seen in FIG. 15B, database 5002B is similar to database 5002A in that database 5002B is in communication with data services 5003 and electronics medical/health records 5006 and can include one or more processors (not shown). Database 5002B is similar to database 5002A in all aspects except that database 5002B can be located at hospital 5020, rather than on a remote cloud server, and that database 5002B can communication with data services 5003 using a healthcare integration API (as shown), rather than HTTPS or REST. Accordingly, because database 5002B resides on premises at hospital 5020, database 5002B can communicate with EMRs 5006 that may not have capabilities to communicate with databases located on a remote cloud server, for example and without limitation due to network firewalls, network policy configurations, and/or lack of VPN capabilities). Additionally, all records received by one or more processors now resides on premises on hospital 5020, therefore mitigating privacy and data integration concerns because records associated with patient ID will not be sent outside hospital 5020.

According to embodiments, as can be seen in FIG. 15B, data services 5003 can directly communicate with EMR 5006, LIS system 5007, or lab instruments manager 5008 thereby eliminating the need for database 5002a and b (as shown in FIGS. 15A-B). In this configuration, one or more of EMR 5006, LIS 5007 and lab instruments manager 5008 can be located on cloud or on-premises. As such, data services 5003 can include one or more processors that perform the same functions as one or more processors on database 5002a as discussed above. Additionally, data services 5003 can communicate with electronics medical/health records 5006 according to a well-defined protocol, for example, not limitation, Fast Healthcare Interoperability Resources (FHIR) standard, such as those disclosed in U.S. Patent Publication No. 20017/0270532, which is incorporated herein in its entirety. In some embodiments, database 5002A can communication with electronics medical/health records 5006 using SMART on FHIR. According to embodiments, data service 5003 can communicate with EMR 5006, LIS system 5007, or lab instruments manager 5008 such that records from EMR 5006, LIS system 5007, or lab instruments manager 5008 can be received using blockchain technologies. According to embodiments, details of suitable digital passes and corresponding verification systems and methods are set forth in U.S. Pat. No. 10,991,158 to Luthra et al., which is incorporated herein in its entirety.

In accordance with the disclosed subject matter, a system for managing patient health, wellness, and more is provided comprising a sensor that is configured to be positioned at least in part in contact with the interstitial fluid in a body of a user. In one embodiment, the system can manage diabetes and use a glucose sensor 104. The system further includes sensor electronics 160 configured to be coupled to the glucose sensor and process data indicative of a plurality of monitored glucose levels from the glucose sensor. The system further includes a network 190 comprising one or more servers and at least one processor configured to receive the processed data and receive or store the processed data in a database such as 5002A or 5002B, wherein the processed data is associated with the user. The database 5002A and 5002B can be on a server, multiple servers, or on a distributed server network such as network 190 including one or more cloud servers. The system further includes a reader device 120 configured to receive the processed data from the sensor electronics and the server 180 receives the processed data from the reader device. The system further includes one or more processors configured to create a blockchain, record on the blockchain the first record, including the first data and the associated personal identification. The one or more processors are further configured to record on the blockchain the second record, the second record including the second data and the associated personal identification. The one or more processors are further configured to access the recorded first record on the first blockchain, pair on the blockchain the first data and the second data based upon a shared data item contained in the first record and the second record.

In accordance with the disclosed subject matter, a system for bi-directional communication of patient data using the blockchain is provided comprising a first database having a first record including first data associated with a personal identification of a patient and a second database having a second record including second data associated with a user identification of the patient. The system further includes one or more processors configured to create a blockchain, record on the blockchain the first record, including the first data and the associated personal identification. The one or more blockchains can be implemented on a server, multiple servers, or on a distributed server network such as network 190 including one or more cloud servers. The one or more processors are further configured to record on the blockchain the second record, the second record including the second data and the associated personal identification. The one or more processors are further configured to access the recorded first record on the first blockchain, pair on the blockchain the first data and the second data based upon a shared data item contained in the first record and the second record.

In accordance with the disclosed subject matter, a method for bi-directional communication of patient data is provided comprising receiving from a diagnostic system, using one or more processors, a first data, receiving from a user, using the one or more processors, a personal identification associated with the first data, creating, using the one or more processors, a blockchain, recording, using the one or more processors, a first record on the blockchain, the first record including the first data and the personal identification associated with the first data, accessing, using the one or more processors, the recorded first record on the blockchain, receiving, using the one or more processors, a second record including a second data associated with a user identification from the second database, pairing, using the one or more processors, the first data and the second data on a block of the blockchain based upon a shared data item contained in the first record and the second record; and displaying, using one or more processors, a combination of the first data and the second data.

By using the blockchain in this manner, the bi-directional communication of patient data will be improved. On the blockchain, the two data records are paired based on a shared data item contained in the first record and the second record. The shared data item is an email address, name and date of birth, a public cryptographic key, or any other unique identifying information. Further in accordance with the disclosed subject matter, the system can include a first database that is an electronic medical record system, and a first data that is laboratory measured HbA1c. The system can further include a second database that is an analyte monitoring system data service, and second data that is glucose levels measured by an analyte monitoring system. The system can further generate a notification based upon the first data paired with the second data, wherein the notification is displayed as the combination of the first data with the second data. Because the system enables the use of FHIR standards, including FHIR extensions embodying a healthcare provider directory (HPD) standard, or H7, the system allows for programmable hooks that could be linked to one or more data sets. The system could further allow these hooks to be programmed using SMART applications to be plugged into the EMR or EHR 5006 system of the provider, including being provided through the EMR or EHR 5006 system. The system can further perform a calculation based upon the first data paired with the second data, wherein the calculation includes a glucose derived A1c, or a personalized HbA1c.

Further in accordance with the disclosed subject matter, the method may include a first database first database that is an electronic medical record system, and a first data that is laboratory measured HbA1c. The method can further include a second database that is an analyte monitoring system data service, and second data that is glucose levels measured by an analyte monitoring system. The method can further comprise generating a notification based upon the first data paired with the second data. The method can further perform a calculation based upon the first data paired with the second data, which can include a glucose derived A1c or a personalized HbA1c. In a further embodiment, the notifications can be directed to a user with requests for reported outcome measures, such as to identify any preceding activities or other factors that could be matched with the first data and second data to provide further insight into the health of the monitored patient. The blockchain in another embodiment is enhanced by associating other types of patient recorded information with the analyte monitoring events. For example, if an identified glucose derived A1c is outside the expected range, a notification can be triggered to direct the patient with a response to ask how the patient is doing, thus providing additional context to help physicians improve management issues that require more patient-directed management.

A further description regarding the blockchain technologies is described herein for illustrative purposes. A blockchain based database allows the storing of records using public and private keys, wherein the private key is unique to a user. An advantage of a blockchain database includes immutable characteristics once the transaction record has been updated on the blockchain. The blockchain database includes a distributed transaction ledger storing information that is accessible by databases 5002A or 5002B. Due to the nature of the decentralized ledger, blockchain transactions are immutable. Confirmed transactions of the blockchain use cryptography to ensure that the integrity of the ledger can be verified by any particular node on the network. Blocks on the blockchain may include a block ID and data content.

As discussed above, database 5002A can receive a record of results from the laboratory analysis along with the associated patient ID. As also further discussed above, each Hospital may generate a different patient ID for results for a patient. Additionally, analyte measurements from data services use an associated user ID, but multiple analyte measurement systems may have different user IDs. These user IDs may not be associated with each other. At the blockchain, each user's record would associate each user ID and patient ID with a user. A user's record at the blockchain thus would have a full listing of every associated user ID and patient ID. The blockchain record will be used to link the results for every patient ID coming from EMR 5006 and every user ID coming from data services 5003. This will allow integration of records for disparate hospitals and analyte measurement services or systems. To allow the patient ID to be linked to a particular user, a request can be made to the user at the hospital to seek consent to share patient identifying information with the blockchain database. In a further embodiment, any provider system could make the request to the patient for authorizing or sharing of the patient generated health data. The request made at the hospital is a non-limiting example of how the patient request can be made to share the patient generated health data.

Non-fungible tokens (NFTs) are unique identification codes that can be used on a blockchain. NFTs are recorded on the blockchain with a unique identifier that distinguishes each NFT. NFTs can associate one or more attributes related to the patient data, including, for example, the patient ID or the user ID. Additionally or alternatively, machine learning technologies and neural networks can be used to associate the one or more attributes related to the patient data. An NFT is generated for a patient that associates the one or more attributes related to the patient data. As discussed above, each user's record on the blockchain would associate each user ID and patient ID with a user. NFTs can be used to perform the association. NFTs minted (the process of generating and assigning the NFT on a blockchain) and assigned to a particular user record contain information unique to the user. Such information can further include a record of results from the laboratory or EMR or EHR 5006 system.

As further discussed above and below, a QR code is used by the share data function of the reader device 120 to generate and display a specially-configured matrix barcode. When using the blockchain, rather than generating a QR code, an NFT can be generated for the share data function. The NFT includes the URL defining the internet location of a web server associated with a report generation system 1860 that can initiate a request to generate a report based on analyte data provided with the request. The NFT can further include an associated patient ID or user ID.

Embodiments disclosed herein include:

A. A system for bi-directional communication of patient data includes a first database having a first record including first data associated with a personal identification of a patient, a second database having a second record including second data associated with a user identification of the patient, and one or more processors configured to: pair the first data and the second data based upon a shared data item contained in the first record and the second record, and display a combination of the first data paired with the second data.

B. A method of bi-directional communication of patient data includes receiving a first data associated with a personal identification, using one or more processors, from a first database, receiving a second data associated with a user identification, using the one or more processors, from a second database, pairing, using the one or more processors, the first data and the second data based upon a shared data item contained in the first record and the second record, and displaying, using one or more processors, a combination of the first data and the second data.

C. A system for managing diabetes includes a glucose sensor configured to be positioned at least in part in contact with interstitial fluid in a body of a user, sensor electronics configured to be coupled to the glucose sensor and to process data indicative of a plurality of monitored glucose levels from the glucose sensor, one or more processors configured to receive the processed data and store the processed data in a health record database, the processed data associated with the user, a first database having a first record including first data associated with a personal identification of a patient, wherein the one or more processors configured to: create a blockchain, record on the blockchain the first record, the first record including the first data and the associated personal identification, record on the blockchain the second record, the second record including the second data and the associated personal identification, access the recorded first record on the first blockchain, and pair on a block of the blockchain the first data and the second data based upon a shared data item contained in the first record and the second record.

D. A system for bi-directional communication of patient data includes a first database having a first record including first data associated with a personal identification of a patient, a second database having a second record including second data associated with a user identification of the patient, and one or more processors configured to create a blockchain, record on the blockchain the first record, the first record including the first data and the associated personal identification, record on the blockchain the second record, the second record including the second data and the associated personal identification, access the recorded first record on the first blockchain, and pair on a block of the blockchain the first data and the second data based upon a shared data item contained in the first record and the second record.

E. A method includes receiving, using one or more processors, a first record including a first data associated with a personal identification from a first database, receiving, using the one or more processors, a second record including a second data associated with a user identification from a second database, pairing, using the one or more processors, the first data and the second data based upon a shared data item contained in the first record and the second record, and displaying, using one or more processors, a report based upon the first data and the second data.

Each of embodiments A, B, C, D and E may have one or more of the following additional elements in any combination: Element 1: wherein the first database is an electronic medical record system. Element 2: wherein the first data is laboratory measured HbA1c. Element 3: wherein the second database includes an analyte monitoring system data service. Element 4: wherein the second data includes glucose levels measured by an analyte monitoring system. Element 5: wherein the shared data item includes an email address. Element 6: wherein the one or more processors is further configured to receive a request to read, write, edit, or delete a resource data in the first or second database, wherein the request is formatted according to a Fast Healthcare Interoperability Resources (FHIR) standard and FHIR extensions embodying a healthcare provider directory (HPD) standard, or H7. Element 7: wherein the one or more processors is further configured to generate a notification based upon the first data paired with the second data. Element 8: wherein the notification is displayed as the combination of the first data paired with the second data. Element 9: wherein the one or more processors is further configured to perform a calculation based upon the first data paired with the second data. Element 10: wherein the calculation includes calculation of a glucose derived A1c. Element 11: wherein the calculation includes calculation of a personalized HbA1c. Element 12: further including a reader device configured to receive the processed data from the sensor electronics, and further wherein the server receives the processed data from the reader device. Element 13: wherein the shared data item includes a public key. Element 14: further comprising a server comprising the one or more processors. Element 15: further comprising a distributed server network comprising the one or more processors.

Element 16: further comprising generating, using the one or more processors, a notification based upon the first data paired with the second data. Element 17: further includes calculating, using the one or more processors, a plurality of personalized glucose metrics using at least one physiological parameter and at least one of the first record or the second record, wherein the first record is laboratory measured HbA1c and the second record is glucose level data indicative of an analyte level of a user, wherein the report comprises a plurality of interfaces including two or more of: the first record, the second record, or the calculated plurality of personalized glucose metrics, and wherein the plurality of interfaces comprising the report are based on a user type. Element 18: wherein the plurality of personalized glucose metrics includes one or more of an adjusted A1c, a calculated A1c, an adjusted calculated A1c, a personalized glucose, a personalized average glucose, or a personalized time in range. Element 19: further comprising calculating a plurality of personalized glucose targets corresponding to the calculated plurality of personalized glucose metrics. Element 20: wherein the plurality of interfaces further includes the plurality of personalized glucose targets. Element 21: wherein the plurality of personalized glucose targets includes one or more of a target glucose range or a target average glucose. Element 22: wherein the personalized target glucose range includes a personalized lower glucose limit. Element 23: wherein the personalized target glucose range includes a personalized upper glucose limit. Element 24: wherein the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant. Element 25: wherein the plurality of interfaces further includes the at least one physiological parameter for the user. Element 26: wherein the user type includes a health care professional. Element 27: wherein the plurality of interfaces includes a glucose monitoring data interface, a glycated hemoglobin interface, a personalized a1c interface, a personalized glucose interface, a personalized average glucose, and a personalized time in range interface. Element 28: wherein the user type includes the user. Element 29: wherein the plurality of interfaces includes a glucose monitoring data interface, a glycated hemoglobin interface, a mean glucose interface, and a time in range interface. Element 30: wherein the plurality of interfaces comprising the report are predetermined based on the user type. Element 31: wherein the plurality of interfaces comprising the report can be selected by the user. Element 32: further comprising outputting a notification if at least one of the plurality of personalized glucose metrics is at or above the corresponding plurality of personalized glucose target. Element 33: wherein the notification comprises a visual notification. Element 34: wherein the notification comprises an audio notification. Element 35: wherein the notification is an alarm. Element 36: wherein the notification is a prompt. Element 37: wherein the reader device wirelessly receives the glycated hemoglobin level for the user from an electronic medical records system. Element 38: wherein the reader device wirelessly receives the glycated hemoglobin level for the user from a cloud-based database. Element 39: wherein the reader device wirelessly receives the glycated hemoglobin level for the user from a QR code. Element 40: the reader device wirelessly receives the glycated hemoglobin level for the user from a home test kit.

As discussed herein, reader devices 120 are often intentionally designed to limit their cost and interoperability. Reader devices 120 often limit processor power and the types of wireless communications hardware included therein. In particular, reader devices 120 are often not configured to communicate on a wide area network and so lack WiFi-compatible hardware. Instead, reader devices 120 are configured only to communicate wirelessly with sensor control devices 102 to preserve security. While multi-purpose data receiving devices 130 (e.g., smartphones, tablets, smartwatches, etc.) are increasing in popularity as secondary options, reader devices 120 used by a large percentage of patients who wear analyte sensors.

To communicate data to other user devices 140 or to an application server 155 (or previously referred to as data services 5003), a physical wired (e.g., USB) connection is established between the reader device 120 and the user device 140. The user device 140 can then optionally relay relevant information to the application server 155. Software executing on the user device 140 or the application server 155 can interpret the received data and, for example, generate actionable reports based on the analyte data included therein.

Analyte reports can be generated by, or on behalf of, a patient or HCP from software executing on the HCP's user device 140 (e.g., a computer terminal in their office) or via a remote, web-based application. For reports to be generated based on up-to-date information from a reader device 120, the patient must periodically connect the reader device 120 to an internet-connected device through a wired connection. However, as described herein, it can be cumbersome and inconvenient to provide the wired connection between the reader device 120 and the user device 140. The user device 140 must have a data interface software driver that can retrieve the data from the reader device 120 and transfer it to the reporting software. When drivers associated with the reader device 120 or the user device 140 are updated, the drivers must be acquired before a secured connection can be established, further delaying report generation steps. Due to a lack of interest or knowledge, many patients do not go through the trouble of creating an account for web-based reporting software, or uploading their latest data via an internet connected device. Similarly, many HCPs do not or create an HCP account in report generating software. Subsequently, for many patients who use analyte sensors, HCPs are not able to make efficient, optimal therapy decisions based on up-to-date information from the user's sensor control device 102.

This problem can be particularly acute for users who do not regularly connect a reader device 120 to their user device 140. This can cause the drivers to facilitate a secured connection between the reader device 120 and the user device 140 to be out of date. For example, non-specialist HCPs can wish to review overall trends in the analyte levels of their patients, but do not frequently see patients who use a reader device 120. The non-specialist HCP can only use a user device 140 to review data from a reader device 120 when certain patients visit. As another example, certain patients can be generally satisfied with using a reader device 120 to review day-to-day information from a sensor control device 102 and therefore only connect their reader device 120 to a user device 140 when requested by an HCP.

HCPs, particularly non-specialist HCPs face a variety of challenges in accessing their patients' analyte data and particularly reports generated based on analyte data. These challenges are a major barrier to the utility of continuous analyte monitors, such as continuous glucose monitors, in informing treatment decisions for patients. Many analyte reporting tools require both the patient and HCP to each register accounts, each requiring a username and password, with an application server 155 associated with the reporting tool. The patient must then enter account credentials in order for data from their reader device 120 to be uploaded to the application server 155 for processing. The patient must also provide permission to the HCP to link their patient record with the HCP account. This requires both parties to remember their account credentials.

To address these barriers, disclosed herein are techniques for enabling transfer of information to assist with report generation from a reader device 120 that do not require a physical data connection with the reader device 120 or an account (for either the HCP or the patient) for report generation software from an application server 155. Techniques directed to alternative methods to retrieve analyte data from a reader device 120 further simply procedures for primary care providers (PCPs) and other HCPs to view analyte data and analyses based on analyte data without requiring the PCP to create a separate account, remember a username and password, or engage additional communication channels to request patients to provide the data. Described herein are a variety of systems and techniques for enabling HCPs to easily access their patients' analyte reports. Of particular benefit is that the systems and techniques described herein do not require the additional overhead of having to manage new or unique accounts and data storage requirements. This allows for the techniques to be used with and tailored to the specific clinical application or electronic medical record (EMR) system. These techniques can be extended to the application of configuring EMR systems to access analyte data where similar challenges exist, such as requiring a patient to remember and provide access credentials to permit an EMR to access a patient's data on a one-time or repeating basis.

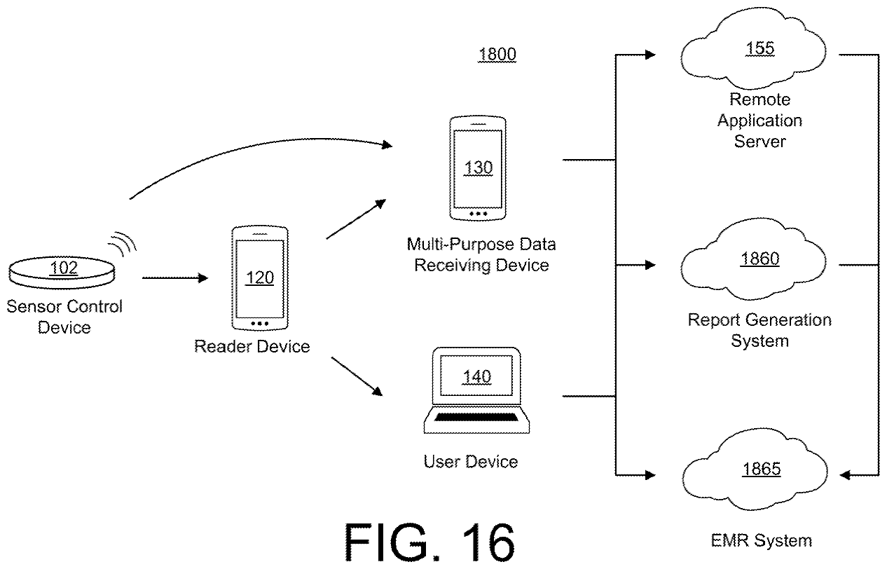

FIG. 16 illustrates an example environment and dataflow for an analyte monitoring system 1800 configured to provide accessible analyte data reports and cross-system integration. The sensor control device 102, configured as described herein, measures levels of one or more analytes of interest of a patient and provides the analyte levels (or signals from which the analyte levels can be derived) to a reader device 120. In certain embodiments, the sensor control device 102 can additionally or alternatively provide the analyte levels to a multi-purpose data receiving device 130.

The reader device 120 is a low-cost proprietary computing device configured to communicate with the sensor control device 102 and provide basic reporting functionality regarding the patient's health. The reader device 120 is generally not capable for wireless communication with other generic devices. The reader device 120 can be connected to multi-purpose data receiving devices 130 or user devices 140 through wired connections or certain limited functionality communication mechanisms.

Multi-purpose data receiving devices 130 and user devices 140 include general purpose computing devices. The computing devices are executing software applications or other bundles of executable programming code that are configured to enable the computing devices to communicate with devices within the analyte monitoring system 1800. Multi-purpose data receiving devices 130 and user devices 140 can typically perform some processing of analyte data to produce certain forms of reports or recommendations. Multi-purpose data receiving devices 130 and user devices 140 can also relay (processed or raw) data from the sensor control device 102 to a variety of remote systems.

The remote systems include, by way of example and not limitation, a remote application server 155 associated with the analyte monitoring system 1800, a report generation system 1860 associated with the analyte monitoring system 1860, and, optionally, an EMR system 1865 (or previously referred to as EMR/EHR 500), which can be integrated with the analyte monitoring system 1860. The remote application server 155 can provide for advanced data processing based on individual patient data or patient data from a larger population. The report generation system 1860 can generate reports based on provided analyte data. The reports can be patient identifying, semi-anonymous, or fully anonymous based on the preferences of the user requesting the generation of the report and the quality of the data. The EMR system 1865 is generally a medical records system administered by or on behalf of an HCP. The EMR system 1865 can, under embodiments described herein, be integrated with the analyte monitoring system 1800 such that it can automatically receive patient analyte data and reports to facilitate treatment and therapy decisions by HCPs.

As described herein, a multi-purpose data receiving device 130 can include a user's smartphone or other device with wide area networking capabilities. The multi-purpose data receiving device 130 can have installed on it an application associated with the analyte monitoring system 100 that enables the multi-purpose data receiving device 130 to communicate with other devices within the analyte monitoring system 100, including the sensor control device 102 and remote application server 155. The application can provide for a variety of additional features.

According to certain embodiments, the application can provide for a feature to facilitate sharing access to analyte data that the multi-purpose data receiving device 130 has uploaded to a remote application server 155 and reports generated based on the analyte data. In a user interface of the application, this feature can be labeled as a “share reports” feature. When the user activates the feature, a unique access code is displayed along with an access URL.

In certain embodiments, the unique access code can be randomly generated on demand and in response to the user's request. In certain embodiments, the application generates the access code, however in other embodiments, the access code is generated by, for example, the remote application server 155 or report generation system 1860 and provided to the application. When the application generates the access code, the multi-purpose data receiving device 130 sends an encrypted message with the access code to the report generation system 1860. In certain embodiments, the encrypted message can include additional information, such as analyte data stored by the multi-purpose data receiving device 130 and received from the sensor control device 102. Alternatively, the multi-purpose data receiving device 130 can include information to identify the patient associated with the multi-purpose data receiving device 130. The report generation system 1860 can use the identifying information to retrieve recent analyte data from the application server 155. The report generation system 1860 stores the access code in association with the analyte data or any pre-generated reports.

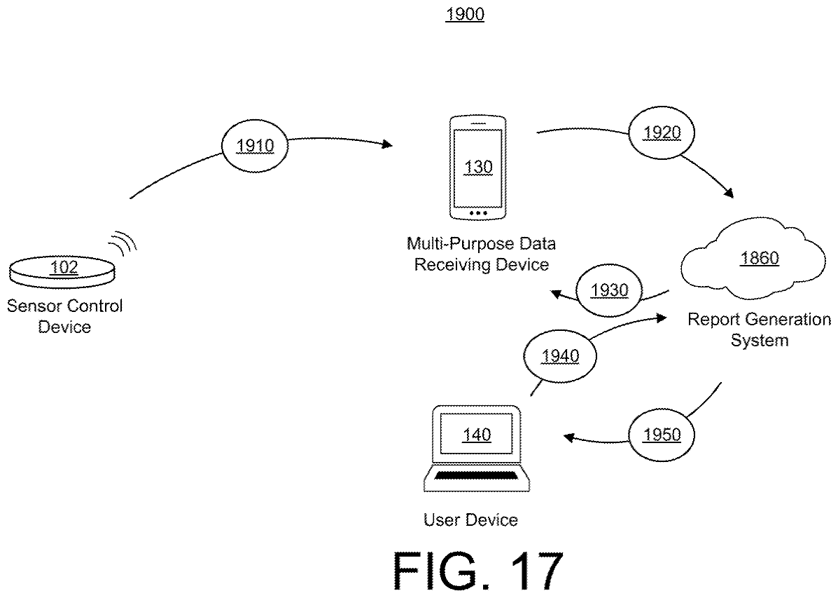

FIG. 17 illustrates an example flow of data between the components of the analyte monitoring system 1900 configured to produce and provide accessible reports. At 1910 the sensor control device 102 provide analyte data to the multi-purpose data receiving device 130. As described herein, the sensor control device 102 can provide the data on a continuous, real-time or near-real-time, basis when a connection between the sensor control device 102 and the multi-purpose data receiving device 130 are available. Additionally or alternatively, the sensor control device 102 can provide the data on demand from the multi-purpose data receiving device 130.

At 1920, the multi-purpose data receiving device 130 initiates a request to share reports based on the analyte data with the report generation system 1860. The request can include the latest analyte data from the sensor control device 102 including a predetermined preceding period of time (e.g., 14 days). The amount of data included can be user set, such as by the patient or their HCP. In particular embodiments, the request can, additionally or alternatively, include a reference to analyte data stored in another system, such as a remote application server 155 of the analyte monitoring system 1800.

Based on the analyte data received from the multi-purpose data receiving device 130, or retrieved from the remote application server 155 at the request of the multi-purpose data receiving device 130, the report generation system 1860 can generate a report for the user. The report can include information such as trends based on the analyte data, trends observed in similar patients or the general population, therapy modification suggestions, and a wide variety of other information. At 1930, the report generation system 1860 can send the report to the multi-purpose data receiving device 130 for viewing by the user of the multi-purpose data receiving device 130. Additionally or alternatively, the report generation system can provide a unique access code and access URL, which can be used by another user of another user device 140 to view the report. In certain embodiments, the multi-purpose data receiving device 130 can generate the access code and provide the access code to the report generation system 1860 during 1920 or during an additional transmission.

At 1940, a user device 140 initiates a request to view the report associated with the analyte data from the sensor control device 102. The user device 140 can navigate, using a web browser or other suitable application, to the access URL. The user of the user device 140 can input the access code to the website, which can be served by the report generation system 1860, a sub-system thereof, or a related website server. The report generation system 1860 can use the access code to identify the analyte data from the sensor control device 102 or pre-generated reports based on the analyte data, if available. The report generation system 1860 can search a database storing the analyte data in association with valid access codes. Upon locating a valid record, at 1950 the report generation system 1860 provides the report to the user device 140 for review.

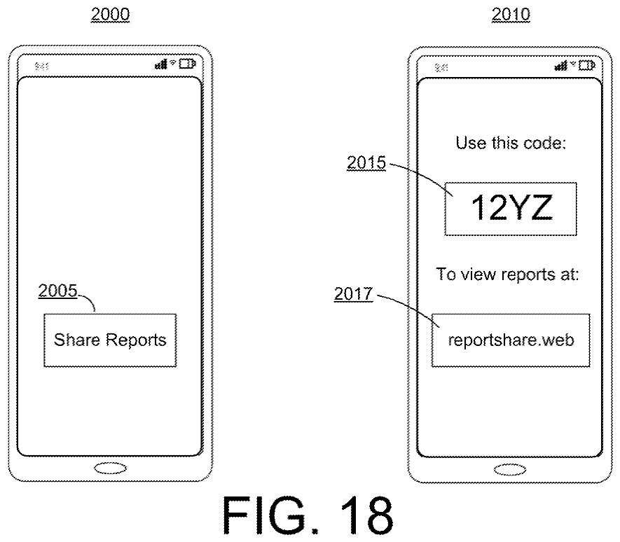

FIG. 18 illustrates example user interfaces of an application executing according to certain embodiments. In particular, FIG. 18 illustrates user interfaces of an application executing on a multi-purpose data receiving device 130. Interface 2000 includes a number of options for accessing data and local reports collected by the multi-purpose data receiving device 130 from a sensor control device 102. Included among the options is an interactive element 2005 (e.g., an interface button) labeled “Share Reports.” When the user selects interactive element 2005, the multi-purpose data receiving device performs the routines described above for collecting analyte data associated with a preceding time period and sending the up-to-date analyte data to a report generation system 1860. The multi-purpose data receiving device 130 also generates, or receives, an access code associated with their reports. Interface 2010 includes a display element 2015 for the access code, in this case “12YZ.” Interface 2010 also includes a display element 2017 instructing a viewer to visit a particular website in order to view the associated reports.

As described herein, in alternative embodiments, the multi-purpose data receiving device 130 can have a constant connection to a remote application server 155 or the report generation system 1860. When the user interacts with the interactive element 2005, the multi-purpose data receiving device 130 can request that data for a relevant period of time be made available. The period of time can be user selected, e.g., based on information included with the request to share reports, or can be automatically determined, e.g., based on the time span of data available about the user. Additionally, in alternative embodiments, the remote application server 155 or report generation system 1860 can generate the access code and send the access code to the multi-purpose data receiving device 130 to confirm that the report is available.

At a later point, the report generation system 1860 receives a request for a report based on patient data from a user device 140. The request includes an access code. The report generation system 1860 searches its records of patient data and/or reports and identifies the corresponding information based on the access code. The report generation system 1860 can then provide the corresponding report to the user device 140 that has requested the report.

FIG. 19 illustrates example user interfaces of another device according to certain embodiments. In particular, FIG. 19 illustrates user interfaces of an application or web browser executing on a user device 140, which can be used by another user such as an HCP. Interface 2100 includes a web browser executing on the user device 140. As illustrated by the address bar 2105, the user of the user device 140 (e.g., the HCP) has navigated to the website provided in interface 2010. The website includes a user input prompt 2107 requesting the user to enter a code to review the corresponding report. After enter the code, e.g., the code shown in interface 2010, the report generation system 1860 receives the code and searches an associated database for the corresponding analyte data or pre-generated report. If there is no pre-generated report for the corresponding analyte data, then the report can be generated on-demand based on the corresponding analyte data. Interface 2110 illustrates an example presentation of the report 2117 for the user of the multi-purpose data receiving device 130. As described herein, the report can include a variety of information and can be presented in a variety of forms based on user preference and the capabilities and features of the user device 140. If there is no report corresponding to the code input by the user, or if the code is no longer valid, interface 2117 can instead show an error indicating that no data is available or that the code has expired.

As described herein, the access code can be limited in terms of the number of uses, the time of use, or other contextual information. As an example, an access code can be set to only be usable for 15 minutes, after which point, the access code will expire. As another example, the access code can be set to only be usable by devices within a specific geographic area or within a specified distance of the multi-purpose data receiving device 130 when the user requests to share their reports. When the other user device 140 submits the request including the access code, the approximate location of the user device can be determined and included with the request. Sensible limitations to when and how often reports can be retrieved improve the security of patient data using the techniques described herein.

In an alternative embodiment, the application executing on the multi-purpose data receiving device 130 has an on-going connection with the remote application server 155 or the report generation system 1860. Through this connection the multi-purpose data receiving device 130 continuously uploads data received from the sensor control device 102 worn by the user. The data is stored in one or more databases for the user. In particular embodiments, the database can store the analyte data in or associated with an account registered by the patient. For example, the patient can register a username or user identifier and password, while can be required to access the data. As another example, the user can store the analyte data in a de-identified fashion using an anonymous account. In such embodiments, the remote application server 155 or report generation system 1860 can recognize an identifier for the multi-purpose data receiving device 130 (e.g., a serial number, IMEI, MAC address, etc.) or a identifier for the application instance of the application executing on the multi-purpose data receiving device 130. The remote application server 155 can associate analyte data from the same recognized identifier in the database. In this embodiment, when the patient requests to share a report, the access code is generated, shared between the multi-purpose data receiving device 130 and the repot generation system 1860, and displayed. The analyte data is not sent from the multi-purpose data receiving device to the report generation system 1860 on-demand.

As described herein, proprietary reader devices 120 can be provided to users in the analyte monitoring system 100. The reader devices 120 can be of generally limited functionally, including the hardware and programming to communicate with the sensor control device 120, but often lacking the ability to communicate using a wide area network. Instead, to upload analyte data to an application server 155 for additional storage and processing, the reader device 120 can be connected to a user device 140 through a wired connection. The user device 140 can store data for the reader device 120 and can further upload the analyte data to the remote application server 155. While the low costs of reader devices 120 can make them convenient to provide to users, the inability to easily transfer data stored on the reader device 120 to another user, such as an HCP, on demand limits the usefulness of the analyte data viewed by the HCP when making therapy decisions. It would therefore be beneficial to provide for simplified processes to transfer analyte data and other information from a reader device 120 to a user device 140 (such as the user device of an HCP in a clinical setting) without a direct wired or wireless connection. It would particularly be beneficial to enable the HCP to view robust reports based on the analyte data, similar to the reports that are available through the above-described “share reports” feature provided through multi-purpose data receiving device 130.

Pursuant to the techniques described herein, the software executing on the reader device 120 can be modified to provide a “share data” function that can be accessed, for instance, from a setup menu. Rather than upload data to a remote server (because the reader device 120 cannot connect with the remote server), when selected by the patient, the share data function causes the reader device 120 to generate and display a specially-configured matrix barcode (e.g., a QR code). This matrix barcode is generated to include a URL defining the internet location of a web server associated with a report generation system 1860 that can initiate a request to generate a report based on analyte data provided with the request. The matrix barcode can also include a sequence of encoded analyte data values that can be provided as parameters passed with the URL.

Special encodings, described herein, enable a large amount of data to be included in the matrix barcode that balances the ability of a user to interact with and use the matrix barcode while still providing sufficient data to provide useful information when converted into a report. For example, analyte value readings could represent values for every 15 minutes for a period of 14 days.

Once the reader device 120 generates the matrix barcode, another user can use a multi-purpose data receiving device 130 to scan the matrix barcode. Many modern smartphones, for example, include a camera that can automatically recognize and decode simple matrix barcodes. The patient or HCP user can use a camera feature native in their smartphone or user device 140 to scan the matrix barcode. The smartphone or user device 140 can direct a web browser to the website server 1860 identified by the encoded URL. The website server 1860 includes a report generation function that decodes the encoded analyte data and generate an analyte report based on these data. The website server 1860 will send the report back to the web browser for display on the multi-purpose data receiving device 130 or user device 140. In certain embodiments, no identifying information is encoded within the matrix barcode, meaning that analyte level report security is non-identifiable and protected through requiring physical access to the matrix barcode to be actionable.

If the HCP wishes to view the report on another device, such as a user device 140, the report generation system 1855 can generate an access code, associate the access code with the analyte data in a database, and display the access code on the multi-purpose data receiving device 130 along with a URL through which the HCP can access the corresponding reports. As in the embodiments described previously, using the user device 140, the HCP visits the report viewing website, provides the access code displayed by the multi-purposes data receiving device 130 and receiving the report from the report generation system 1860.

In certain embodiments, for added security, the website server 1860 can generate the access code and include this in the glucose report that is displayed on the multi-purpose data receiving device 130 along with another URL identifying another website. The access code can be, for example, a random alphanumeric code. The HCP can access this second website from a web browser on their internet-connected user device 140 or any other internet connected devices by entering the URL into the browser URL field. The second website can also be provided by the same provider as the first website. The second website can request the HCP to enter the access code by causing a code entry field to be displayed on user device 140 web browser. Upon entering the code and indicating submit, the second website will receive the access code and generate analyte report(s) with the patient's data for display on the HCP's user device 140 web browser.

In some embodiments, the report generation system 1860 can generate the access code for the first and second websites. In some embodiments, the access code can be generated by the reader device 120, e.g., as part of the process for generating the matrix barcode. The access code can be associated with reports (if generated upon receiving the analyte data) or analyte data used for generating reports on demand. The access code can be used on a permanent or semi-permanent basis by the user of the reader device 120, such as to facilitate consistent sharing of data. Alternatively, the access code can be temporarily associated with the reports or analyte data. The association can be removed after the expiration of a predetermined or user-provided amount of time, after the report has been accessed a specified number of times (e.g., to facilitate sharing the same report with multiple other users), or upon request by the user of the reader device 120 or the user who has accessed the report. The code can take any form, for instance a sequence of alphanumeric digits such as “AB12,” or can include other symbols, or pictograms (e.g., emojis).

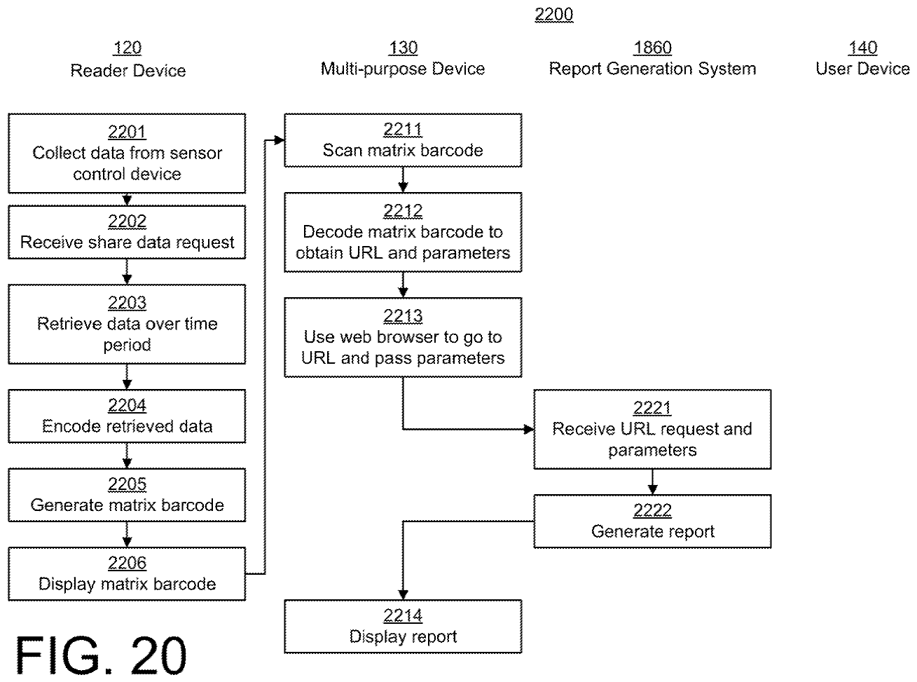

FIGS. 20 and 21 illustrate a first method 2200 and a second method 2300 for requesting and generating a report based on analyte data provided via a matrix barcode. The process begins at 2201, when the reader device 120 collects analyte data from a sensor control device 102.

At 2202, the reader device 120 receives a request to share or offload analyte data. The request can be made, for example, through a user of the reader device 120 selecting an interactive element provided on a user interface of the reader device 120.

At 2203, the reader device 120 retrieves analyte data associated with a particular time period of interest. The reader device 120 can be configured to automatically identify data for a predetermined period of time (e.g., the last 14 days, 7 days, 24 hours, etc.). In particular embodiments, the request received at 2202 can specify the time period of interest.

At 2204 the reader device 120 encodes the retrieved data. Prior to generating the matrix barcode associated with the analyte data, the analyte data can be encoded. Encoded analyte data can be achieved by formatting the data to be compatible with the parameters provided to the report generation system 1860 so that the report generation system 1860 can generate the report. As an example, the encoded data can be made up of a date stamp and/or timestamp associated with each analyte data value associated with the analyte data. The encoded data can include a single date stamp and/or timestamp followed by a series of analyte data values that are interpreted as being provided at regular intervals (e.g., every 5 minutes, 15 minutes, 30 minutes etc.). This encoding reduces the need to include explicit timing information for each analyte data value when not necessary.

When generating matrix barcodes, the complexity of the barcode scales with the density of the data included in the matrix barcode. The more information, particularly the number of bytes, one attempts to include in the matrix barcode, the more complex the barcode becomes. The size of the matrix barcode can be increased to enable more information, but at a certain point, the limit is based on the display resolution of the device displaying the matrix barcode (e.g., the reader device 120) and the fidelity of the camera used to scan the matrix barcode. Therefore, additional encodings can be used to improve the balance of information density in the barcode.

One method to reduce the information density is to reduce the period of time that is covered by the analyte data included with the matrix barcode. Another method to reduce the information density is to reduce the frequency of the analyte data within the series (e.g., analyte data values corresponding to every 30 minutes instead of every 15 minutes). These solutions are simple and do not require additional computational power or time to use. However, they also reduce the amount of useful data provided to the user who views the ensuing report (e.g., the HCP).

To reduce the information density but maintain parameters such as the duration and frequency of the included data, different forms of data compression can be used. As one example, the dynamic range (e.g., precision) of the analyte data values can be reduced in a context aware fashion. One such data compression method can be to round the analyte data values. For example, analyte data values can be stored by the data receiving device with precision up to 0.1 mg/dL. Before encoding the data values into a matrix barcode, the data can be rounded or truncated to a precision of up to 1 mg/dL. As another example, analyte data values can be rounded to the nearest 5 mg/dL (or 10 mg/dL or other appropriate values).

In particular embodiments, rounding can be performed to round different levels of precision based on the original value of the analyte data value itself. Specifically, for an analyte where values higher than a threshold are considered more important to monitor than values below the threshold, analyte data values above the threshold can be rounded to a more precise value. The reverse can be true for analyte data values where lower values are considered more important to monitor. Multiple thresholds and multiple levels of rounding precision can be used to scale the rounding precision accordingly. Additionally, the threshold values can be pre-programmed or can be set by the user of the reader device 120 or the user who reviews the reports (e.g., an HCP) based on the individual user's need or preferences.

As an example, where the analyte is or correlates to glucose values, it can be preferable to have more resolute glucose values for lower glucose values than for higher glucose values. When decoding the glucose series, approximate resolution to 1 mg/dL could be partially recovered by using spline techniques on the series and rounding the resulting glucose values to the nearest 1 mg/dL.

An example of a multi-threshold rounded scheme is given below:

For glucose values from 40-100: round to nearest 2 mg/dL

For glucose values from 101-180: round to nearest 3 mg/dL

For glucose values from 181-280: round to nearest 4 mg/dL

For glucose values from 281-400: round to nearest 5 mg/dL

When rounding the analyte data used in the matrix barcode metrics calculated by the report generation system 1860 for inclusion in the report are calculated using the rounded data. The values of metrics included in reports calculated using analyte values with a higher precision can differ from values of metrics calculated using analyte values with a rounded precision. Therefore, the value of the metrics shown in a report can differ slightly from the values that are presented on the reader device 120. This can cause the user or HCP to question the accuracy of the data, be distracted by the difference, or even to change therapy recommendations based on the less accurate data. To address this issue, the input data to the matrix barcode can further include metrics calculated based on the original analyte data (e.g., with full resolution) to be used in subsequently generated reports. The report generation system 1860 can use these metrics instead of recalculating them from the rounded glucose data. This has the added advantage of allowing the report generation system 1860 to execute more efficiently because it does not need to calculate the metrics before the report can be generated.

The report generation system 1860 that receives the web request based on the encoded data will interpret the data as necessary. In particular embodiments, the web request (and therefore the matrix barcode) can include an identifier for the encoding. The identifier can be, for example, a version number that allows the report generation system 1860 to select the appropriate decoding procedure based the version number. Including the identifier provides for added flexibility for reader device 120 programming, would allow for multiple types of encodings to be used simultaneously, and would allow for this procedure to be used even if the user of the reader device 120 has not update the software or firmware executing thereon. The type of encoding can further be selected based on the type of analyte data, the density of the data included in the request, or user preference.

Returning to the method 2200 of FIG. 20, the method 2200 continues at 2205, when the reader device 120 generates the matrix barcode. The matrix barcode can be generated using available algorithms or methods (e.g., the Quick Response (QR) code) or can be generated by a proprietary algorithm used by the analyte monitoring system 1800. While a proprietary algorithm can be used to improve the security of the process and can enable additional features with the matrix barcode (e.g., including more data), it will also limit interoperability and the convenience afforded by using a more common approach.

At 2206, the reader device 120 displays the matrix barcode. The reader device 120 can further display instructions for using the matrix barcode to request and retrieve the report.

At 2211, the matrix barcode is scanned. As an example, a multi-purpose device 130 can scan the matrix barcode. Another user device 140 having suitable hardware (e.g., a camera) and software to decode the matrix barcode can also be used. Many camera applications of modern smartphones include the capability to easily recognize and scan matrix barcodes.

At 2212, the matrix barcode is decoded to obtain the URL and parameters to be used with the URL. As with scanning matrix barcodes, many modern smartphones include the capability to decode and interpret commonly-available matrix barcodes. If a proprietary algorithm is used to generate the matrix barcode, the multi-purpose data receiving device 130 can be provided instructions to download or execute programming code to decode the matrix barcode. For example, an application associated with the analyte monitoring system 1800 can be downloaded and executed on the multi-purpose data receiving device. Generally speaking, decode the matrix barcode includes identifying the data encoded within the matrix barcode. For example, where the matrix barcode includes the access URL and analyte data, decoding the matrix barcode involves converting that information into plaintext or another format suitable for interpretation by the multi-purpose data receiving device 130 as it determines how to handle the data.

At 2213, the URL and parameters are used to generate and send a web request. For example a web browser executing on the reader device 120 can be used. Having decoded the matrix barcode, the access URL and analyte data to be provided as parameters for the web request to generate a report are available to the multi-purpose data receiving device 130. The multi-purpose data receiving device 130 can construct the web request using standard mechanisms. The multi-purpose data receiving device 130 initiates the request to the report generation system 1860.

At 2221, the report generation system 1860 receives the web request based on the URL and parameters. At 2222, the report generation system 1860 generates the report. As discussed herein, the report generation system 1860 can interpret the parameters passed by the multi-purpose device 130 to discern the analyte values that will be used in the report. The report can take on a variety of forms, including display of individual analyte data values, trend information (e.g., short and long-term extrema, time in range, rate of change trends, etc.), treatment summaries, therapy recommendations, and other suitable information. The content of the report is based on the data available to the report generation system 1860. Wherein the data is generally of low quality or anonymous, the report will necessarily be more limited than when the data is high quality and personalized to the patient. The report generation system 1860 can then send the report back to the multi-purpose device 130. As an example, the report can be provided as a formatted web page or transportable document (e.g., a PDF).

At 2214, the report is displayed by the multi-purpose data receiving device 130.

FIG. 21 illustrates a related method 2300 for requesting and generating a report based on analyte data provided via a matrix barcode. In particular, operation 2321 is an alternative version of operation 2221 illustrated in FIG. 20. Operations 2201-2206 and 2211-2213 illustrated in FIG. 20 can be similarly performed prior to 21, and are excluded only for the sake of providing a concise explanation of the method 2300.

At 2321, the report generation system 1860 receives the web request based on the URL and parameters. The web request includes a request to enable limited access to additional user devices 140. At 2322, the report generation system 1860 generates the report based on the received parameters. The report generation system 1860 uses processes described herein to generate the report based on available analyte data.

At 2223, the report generation system 1860 generates an access code for the report. As described herein, an access code can be associated with the analyte data or the report, once generated, to limit access of the report only to individuals with the access code. Additionally, the report can be used to share the report and analyte data with users and systems without requiring a connection directly between the multi-purpose data receiving device 130 and the other systems. Instead, the report generation system 1860 uses the access code to validate access to the report and provide the report to the other system on behalf of the patient.

At 2224, the report generation system 1860 associates the access code with the report. For example, the report generation system 1860 can store the access code in a database with the report. In embodiments in which the report is generated on demand to display, the database can instead store the analyte data passed as parameters to the report generation system 1860 in association with the access code. As described above, the access code can be a temporary access code that is deleted after certain conditions have transpired such as a number of uses, passage of a specified amount of time, etc.

After the report generation system 1860 generates the access code, the report generation system 1860 can send the access code to the multi-purpose device 130 that initiated the web request at 2213. For example, the access code can be presented as a response to the web request.

At 2315, the access code can be displayed by the multi-purpose device 130. Additionally, the multi-purpose device 130 can be displayed with an access URL through which the report can be accessed. Note that in certain embodiments, the multi-purpose purpose data receiving device 130 can generate the access code itself and include the access code in the request to generate the report issued to the report generation system 1860.

At 2331, another user device 140 can use a web browser to visit the access URL. A web page can be displayed requesting the user of the user device 140 to enter the access code. The access URL can be publicly available and any web browser can be used to visit the access URL because without an access code, visitors to the access URL cannot retrieve patient data. Additionally, in embodiments where the data included in reports shared through the mechanisms described herein include only anonymized or de-identified data, individual patient information is not at risk. If a patient has allowed for personal information to be included, the report can be further protected through passwords or other contextual information limiting access to the report. This can reduce the efficacy of brute force approaches to guess access codes and retrieve patient information.

At 2232, the user device 140 can receive the access code as input to the web page. The user device 140 can send another web request to the report generation system 1860 (or another related server) with the access code.

At 2225, the report generation system 1860 can retrieve the report based on the access code. After receiving the access code, the report generation system 1860 can search the associated database using the access code and can identify the report accordingly. If the access code cannot be located or has expired, the report generation system 1860 can send a message to the user device 140 indicating that an error has occurred and identify the source of the error. If the access code is located and valid, the report generation system 1860 can send the report to the user device 140.

At 2333, the user device displays the report. The report can be display in the web browser or using an application configured to display the report. In some embodiments, the report can be best viewed using a proprietary application associated with the analyte monitoring system 1800, which can be installed on the user device 140.

Although the above examples have been described in the context of a reader device 120, similar techniques can be used with multi-purpose devices 130 configured to receive and process data from second control devices 102. Even though multi-purpose devices 130 can include hardware to connect to wide area networks (including the internet), users can lack have time or motivation to ensure that the remote application server 155 has up-to-date information before visiting an HCP. Similarly, users can not have accounts set up through the remote application server 155 or enabled to share analyte data with an HCP. Additionally, the multi-purpose device 130 cannot have access to the wide area network (e.g., not have cellular service) when visiting an HCP to upload the most up-to-date information. Embodiments described herein preclude the need for patients and/or HCPs to initiate and maintain an account with the report generation software or maintain consistent connection to the remote application server 155 or report generation system 1860.

FIG. 22 illustrates example user interfaces of an application executing according to certain embodiments. In particular, FIG. 22 illustrates user interfaces of an application executing on a reader device 120 without wide area networking capabilities. Interface 2400 includes a number of options for accessing data and local reports collected by the reader device 120 from a sensor control device 102. Included among the options is an interactive element 2405 (e.g., an interface button) labeled “Share Reports.” When the user selects interactive element 2405, the reader device 120 performs routines, including those described herein, for collecting and encoding analyte data associated with a preceding time period. The reader device 120 also generates a matrix barcode including the encoded analyte data and a URL to cause a multi-purpose reader device 120 to upload the encoded analyte data to a repot generation system 1860. The reader device 120 then displays the matrix barcode in an interface, such as interface 2410, which provides instructions for how to initiate the upload.

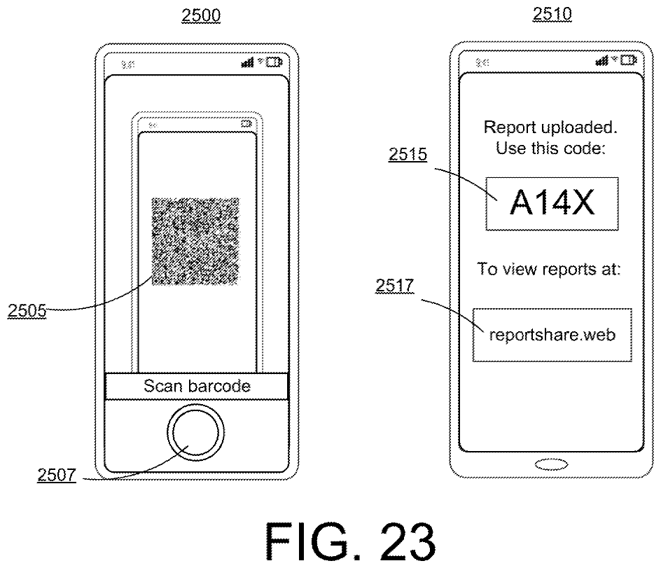

FIG. 23 illustrates example interface of an application executing according to certain embodiments. In particular, FIG. 23 illustrates user interfaces of applications executing on a multi-purpose data receiving device 130. Interface 2500 corresponds to a camera application of the multi-purpose data receiving device 130. The user has positioned the multi-purpose data receiving device 130 so that the reader device 120, while showing interface 2410, is within view of the camera of the multi-purpose data receiving device 130. Using the interactive element 2507, the user of the multi-purpose data receiving device 130 scans the matrix barcode shown in the viewfinder. After the multi-purpose data receiving device 130 scans the matrix barcode, the multi-purpose data receiving device 130 decodes the matrix barcode to interpret the URL and analyte data encoded therein. The multi-purpose data receiving device 130 then initiates a web request based on the URL to upload the analyte data to the report generation system 1860. The report generation system 1860 stores the analyte data in a database and generates an access code associated with the analyte data. The report generation system 1860 provides the access code and an access URL to the multi-purpose data receiving device 130. Interface 2510 illustrates the interface shown after the user has successfully uploaded the analyte data. The interface 2510 includes a display element with the access code 2515 provided by the report generation system 1860 and a display element with an access URL. If a user visits the access URL and provides the access code, the user device 140 used to visit the access URL can provide interfaces such as those illustrated in FIG. 19.

Electronic Medical Record (EMR) systems are used by many HCPs. The EMR system (e.g., EMR system 1865) stores data relating to a patient and the patient's interactions with an HCP. In particular embodiments, it would be beneficial to incorporate systems and techniques, including those described herein, for generating a report relating to a patient's analyte data. In particular, it would be beneficial to simplify the processes used by the HCP and by the EMR system 1865 itself to access the patient's analyte data. For example, an analyte reporting application as described herein can be included as part of the EMR system. The analyte reporting application can be any software program established on the HCPs computer or established on a server that is accessible to the HCP and, in certain embodiment, is not necessarily an EMR.

In many systems, patient data can only be accessed by an EMR system 1865 for storage and processing with explicit permission from the patient. The patient must log into their account with a remote application server 155, identify the correct HCP who needs to see their analyte data, and ensure that the data provided is up to date. In some cases, the HCP must also log into an account with the remote application, identify the correct patient, and issue a request before the patient can authorize sharing data. An HCP can also need to remind a patient to approve a sharing request. This process is slow and error-prone—whether users forget their account credentials, identify the incorrect patient or HCP, or commit other errors—and generally inefficiently uses time when an HCP is considering a patient's medical records and treatment options. Attempts have been made to automate some steps of this process. For example, EMR systems 1865 and remote application servers 155 can attempt to match information like patient name, unique identifier, or contact information to generate a request to authorize data sharing. However, this requires patients to ensure that the information provided to their HCP and the remote application server 155 to be the same, any misspellings or, e.g., alternative contact information, can revert the patient back to a fully manual process. Even when matching is successful, the patient still must log into their account with the remote application server 155 to authorize the sharing request. Moreover, previous attempts to automate data sharing could not account for patients who did not have an account with the remote application server 155.

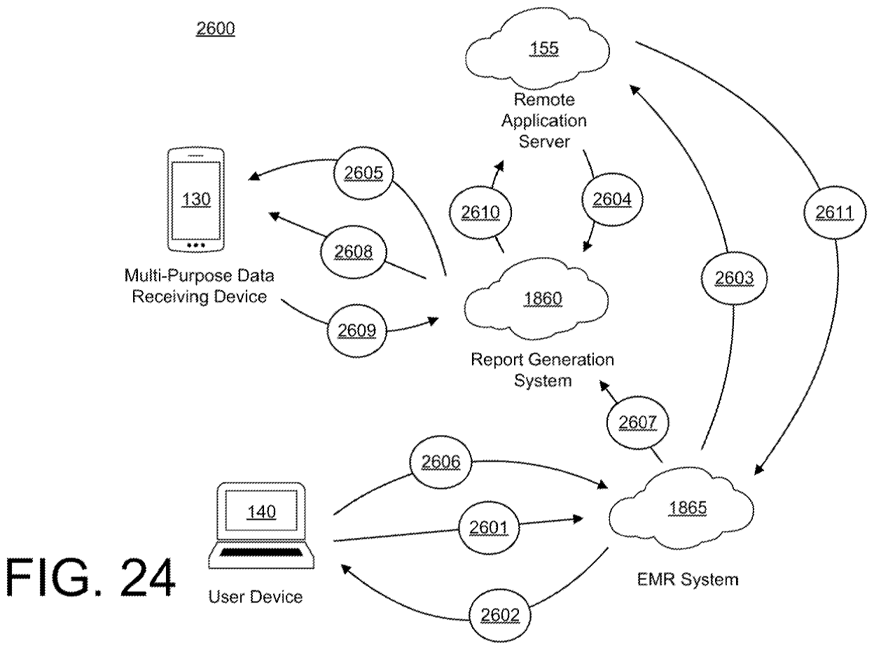

FIG. 24 illustrates an example flow of data between the components of the analyte monitoring system 2600 configured to facilitate a persistent connection between the remote application server 155 and EMR system 1865. The illustrated data flow begins at 2601, in which the HCP, using a user device 140 requests a report from the EMR system 1865 based on or using the analyte data, from remote application server 155, of a patient. The HCP can also initiate a request to review the latest analyte data of the patient or otherwise create a request that requests an interface between the EMR system 1865 and the remote application server 155.

Upon receiving the request, the EMR system 1865 determines that it does not have access to the analyte data of the patient stored on the remote application server 155. The request can include a unique identifier for the patient (e.g., a patient identification number or other unique representation). In certain embodiments, the EMR system 1865 can use the unique identifier for the patient to query a database associated with the EMR system 1865 and stored data on patients who have integrated their data from the remote application server 155 with the EMR system 1865. In certain embodiments, the EMR system 1865 can query the remote application server 155 with a request for data and reports associated with the patient identifier. When the integration between the remote application server 155 and EMR system 1865 has not been completed for a particular patient, the EMR system 1865 will receive a rejection to the request to access the data and reports.

Upon determining that the EMR system 1865 is not integrated with the remote application server data for the particular patient, at 2602, the EMR system 1865 responds that the HCP does not have access to the request information for the patient. The user device 140 can display an error message indicating that access has not been granted to the HCP and/or EMR system 1865. In certain embodiments, the EMR system 1865 can cause the user device 140 to display instructions to the HCP for how to request access and/or integrate the EMR system 1865 and remote application server 155. As an example, the user device 140 can display, through the EMR system application, a prompt for the HCP to enter an access code associated with the patient data.

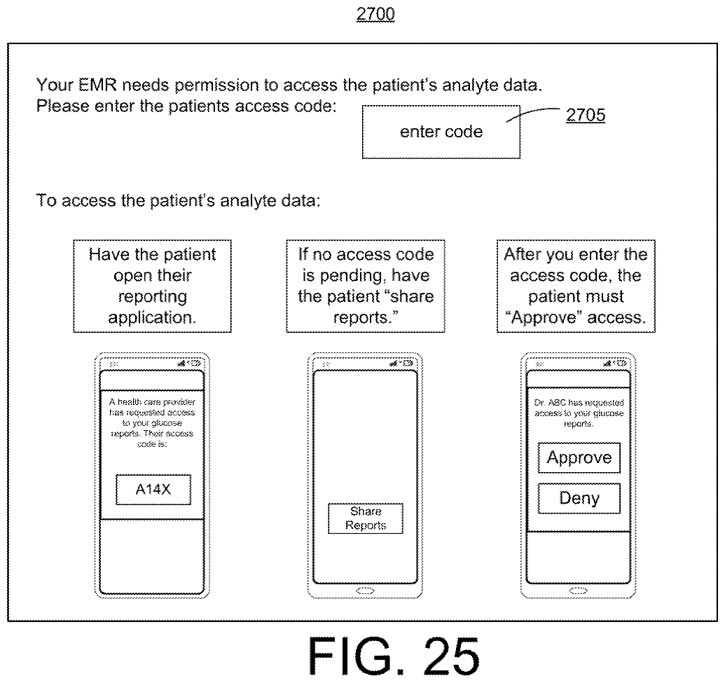

FIG. 25 illustrates an example interface of the user device 140 executing an application associated with the EMR system 1865 showing instructions on how to request access to the analyte data of a particular patient. The interface includes a user input field 2705 through which the HCP can input an access code provided by the patient.

Returning to the dataflow in FIG. 24, at 2603, the EMR system 1865 initiates a request to receive patient data associated with the patient identified by the HCP. The request can include the unique patient identifier and other information for the remote application server 155 to identify which patient is of interest to the HCP. The remote application server 155 can perform validation and integrity checks on the request to reduce the opportunity for a malicious user to spam authorization from the patient.

At 2604, upon receiving the request from the EMR system 1865, the remote application server 155 can request the report generation system 1860 to initialize the integration process on behalf of the EMR system 1865. As described herein, the integration process include generating an access code, providing the access code via the multi-purpose data receiving device 130 of the patient, and allowing the user device 140 to provide the access code to the repot generation system 1860. Therefore, the report generation system 1860 can generate a request to the multi-purpose data receiving device 130. The request can include an indication that an HCP has requested access to the analyte data of the patient associated with the multi-purpose data receiving device 130. If available, e.g., if provided by the EMR system 1865, the request can identify the HCP. The request can also include an access code generated by the report generation system 1860. The EMR system 1865 provides the request to the multi-purpose data receiving device 130 at 2605.

Upon receiving the request from the report generation system 1860, the multi-purpose data receiving device 130 can display the access code. An application associated with the remote application server 155 can launch into foreground execution by the multi-purpose data receiving device 130 and inform the user of the multi-purpose data receiving device 130 (e.g., the patient), that an HCP has requested access to their analyte data. The application can display the access code and provide instructions for the patient to display the access code to their HCP. In certain embodiments, the multi-purpose data receiving device 130 can first confirm that the patient wishes to share their analyte data and reports with the HCP before displaying the access code. In certain embodiments, the multi-purpose data receiving device 130 can generate the access code itself, in which case the multi-purpose data receiving device 130 also provides the access code to the report generation system 1860 in an un-illustrated step.

In an alternative embodiment, the patient initiates the process to generate an access code, rather than the process being initiated by the report generation system 1860 on behalf of the HCP. As discussed herein, the patient can use a “share reports” feature to generate an access code shared between the multi-purpose data receiving device 130 and the report generation system 1860. The report generation system 1860 can use the access code, as discussed below, to identify a patient's records.

The patient provides the access code to the HCP. The HCP enters the access code into the provided user input on the application executing on the user device 140 and associated with the user EMR system 1865. At 2606, the user device 140 provides the entered access code to the EMR system 1865.

At 2607, the EMR system 1865 provides the access code to the report generation system 1860. For the sake of brevity, this is shown as a direct transmission between the EMR system 1865 and the report generation system 1860, although it should be understood that the access code can be provided by the EMR system 1865 to the report generation system 1860 via the remote application server 155. Similarly, in certain embodiments, the user device 140 can have or establish a connection to the report generation system 1860 and the user device can provide the access code to the report generation system 1860 through said connection. As an example, the multi-purpose data receiving device 130 can, in addition to displaying the access code, display an access URL associated with the report generation system 1860. The HCP can use the user device 140 to navigate to the website accessible via the access URL and provide the access code to the report generation system 1860 through the website.

Upon receiving the access code, the report generation system validates and verifies the access code from the user device 140 with the access code generated by the report generation system 1860 (or by the multi-purpose data receiving device 130 and provided to the report generation system 1860). Validating the access code can include determining that the access code has not expired and does not have other restrictions limiting the availability of the access code to permit analyte data integrations. If the access code is invalid, the report generation system 1860 can optionally notify the user device 140 (e.g., through the remote application server 155 or EMR system 1865 as an intermediary system) that the access code that has been provided is not valid or that some other error has occurred. The report generation system 1860 can also notify the multi-purpose data receiving device 130 of an error.

If the access code is valid, the report generation system 1860 can perform an additional access validation with the multi-purpose data receiving device 130. In certain embodiments, the process illustrated in dataflow 2600 can be used to create a continuous or permanent integration of the patient's analyte data with the EMR system 1865. Because this can include highly sensitive information, the analyte monitoring system 1800 can request the patient to validate the request, even after the HCP has provided an access code. This acts as a second factor for authenticating access to the patient data. At 2608, the report generation system 1860 transmits the validation request to the multi-purpose data receiving device 130.

The multi-purpose data receiving device 130 receives the validation request and displays the request to the patient. As an example, the validation request can be displayed as a pop-up or push notification on the multi-purpose data receiving device 130. The notification can include a user interface with a prompt asking the user to validate the access request made by the HCP. The notification can include information associated with the HCP and the EMR system 1865 provided when the EMR system 1865 provided the access code to the report generation system 1860. At 2609, the multi-purpose data receiving device 130 provides the patient's response to the prompt to the report generation system 1860.

If the patient denies access through the prompt, the report generation system 1860 can deny access to the EMR system 1865. The report generation system 1860 can inform the EMR system 1865 that access to the patient's analyte data was not granted. In particular embodiments, the report generation system 1860 can measure the number of requests for access for a particular patient or made by a particular EMR system 1865 or HCP over a period of time and determine to block further access requests if abuse of the system is suspected.

If the patient affirmative responded to the validation request, at 2610, the report generation system 1860 informs the remote application server 155 that access has been authorized. The report generation system 1860 can notify the remote application server 155 associated with the analyte monitoring system 1800 that a valid access code has been received and that the multi-purpose data receiving device 130 has verified the request.

At 2611, the remote application server 155 grants access to the requested patient analyte data and reports and provides the requested data and reports to the EMR system 1865. As described, the integration of data can enable to the HCP to retrieve the latest available analyte data for the patient using the HMR system 1865. For example, as a sensor control device 102 worn by the patient provides data to the multi-purpose data receiving device 130 and the data is uploaded to the remote application server 155, the data can be shared with or accessed by the HCP through the EMR system 1865. The patient can control which data is shared, such as enabling only a one-time sharing of data or sharing data for a particular time range or for a particular period of time. Of particular benefit is that this system can proceed without any user (e.g., the patient or HCP) being required to remember access credentials for any of the systems being used. Additionally, the identity of the patient and HCP are verified to each other multiple times throughout the process, which helps to ensure that the correct data about the correct patient is being shared to the correct HCP.

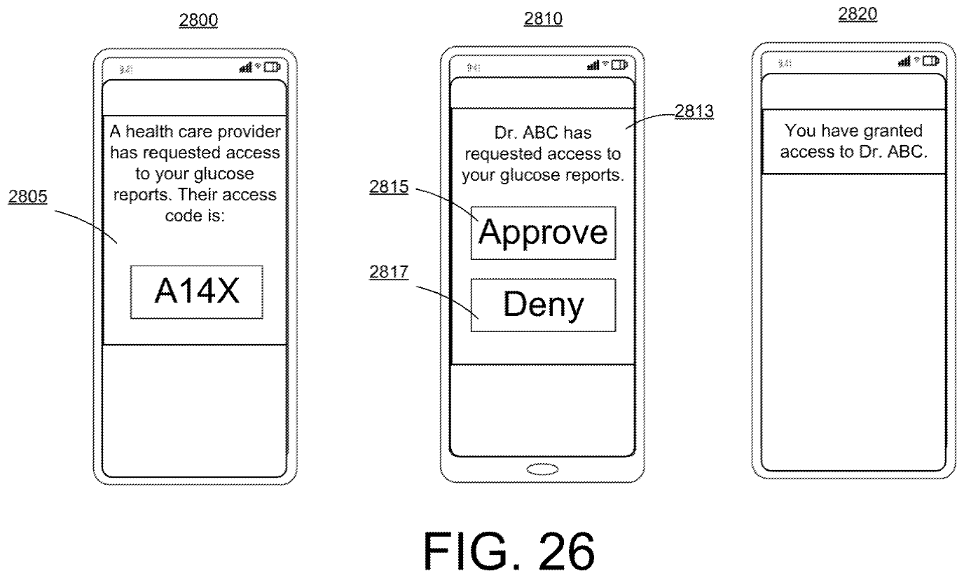

FIG. 26 illustrates user interfaces that can be displayed on a multi-purpose data receiving device according to embodiments disclosed herein. In particular, FIG. 26 illustrates detailed views of user interfaces 2800-2820 that can be used to facilitate the integration of analyte data from the remote application server 155 being shared with an EMR system 1865. User interface 2800 includes a notification 2805 from the application associated with the analyte monitoring system 1800 executing on the multi-purpose data receiving device 130 indicating that an HCP has requested access to the patient's analyte data. The notification 2805 includes an access code that will be used by the HCP as described above. Interface 2810 illustrates a notification 2813 from the application associated with the analyte monitoring system 1800 executing on the multi-purpose data receiving device 130 indicating that a specific HCP has entered an access code. The notification 2813 includes an interactive element 2815 to approve or authorize the access and an interactive element 2817 to deny the access. Interface 2820 illustrates a notification confirming that access has been granted.

Once the connection between the EMR system 1865 and the remote application server 155 is established, information entered by the HCP, e.g., based on the reports from the report generation system 1860, can be shared back to the remote application server 155 as appropriate. For example, therapy modifications or requests for further monitoring performance can be made by the HCP through the EMR system 1865. This can be particularly beneficial where the HCP is not comfortable using native applications provided by the analyte monitoring system 1800 or otherwise prefers their own EMR system 1865 for its convenience. The further information from the HCP can also be shared with the patient through their own access to the remote application server 155 or other applications associated with the analyte monitoring system 1800. Additionally, because analyte data associated with the patient is centralized by the remote application server 155 but can come from sensor control devices 102 via a variety of pathways (e.g., through a reader device 120, multi-purpose data receiving device 130, or user device 140), the data can be shared with the EMR system 1865 regardless of the source. The patient does not need to remember to perform additional steps to ensure that their HCP is provided with up-to-date data to assist with their health monitoring.

In certain embodiments, the integration between the remote application server 155 and the EMR system 1865 can be limited based on system settings or user preference. For example, when granting access, the patient can specify that only analyte data of a certain type or for a specified period of time will be shared with the EMR system 1865. Requests to access data stored by the remote application server 155 and exceeding the granted authorization can be denied by the remote application server 155 on behalf of the patient or can be converted into a request for additional access on behalf of the HCP.

Certain embodiments can facilitate data sharing between the patient and the EMR system 1865 even when the patient does not have an account with the remote application server 155. For example, when the patient requests to “share reports,” the remote application server 155 can generate a limited-use account that is associated with the application instances of the application associated with the remote application server 155. Once the authorization process is completed, the remote application server 155 can share the data associated with the limited-use account. The patient can be provided the option to create a full account to maintain the connection to the EMR system 1865. The patient can also cause the account to be deleted, in which case the data sharing is a one-time operation.

Note that, although the remote application server 155 and the report generation system 1860 are described as separate entities, this is merely for the purpose of providing a clear explanation of the functionalities of various components. In various embodiments, the remote application server 155 and report generation system 1860 can be functions of the same overall system.

In an alternative embodiment, the connection between the remote application server 155 and the EMR system 1865 can be facilitated using a matrix barcode rather than requiring entry of an access code at the user device 140. In this embodiment, the user device 140, upon receiving confirmation from the EMR system 1865 that the HCP does not yet have access to the request patient data, displays a matrix barcode. The matrix barcode can be configured to initiate a web request to enable access of a specific user or system (e.g., the HCP and EMR system 1865) to the patients analyte data stored by the remote application server.

The patient can use a camera of their multi-purpose data receiving device 130 to scan the matrix barcode displayed on the user device 140. The matrix barcode can include instructions to enable authorization for the EMR system 1865 to receive access to the patients' analyte data via the remote application server 155. When the matrix barcode is scanned, the multi-purpose data receiving device 130 can extract details about the authorization request encoded within the matrix barcode and use this information to begin an authorization procedure, such as that described herein. In certain embodiments, the matrix barcode scanning functionality can be bundled into an application associated with the analyte monitoring system 1800 executing on the multi-purpose data receiving device 130 (e.g., that enables the multi-purpose data receiving device 130 to communicate with the sensor control device 102). Scanning the matrix barcode can cause a prompt to allow access to be displayed within the application with information about the type of access that will be granted, who and what system are requesting access, and mechanisms to control access. Upon the patient's affirmative response, access is granted to the EMR system 1865, which can be notified by the remote application server 155.

As discussed herein, if the patient affirmatively initiates the request to generate an access code, e.g., through activating a “share reports” feature of the application executing on the multi-purpose data receiving device 130, interface 2800 can merely confirm that recent analyte data has been provided to the report generation system 1860 and provide the access code.

As discussed herein, reader devices 120 are lower cost mechanisms for a patient to interact with an analyte monitoring system 1800. Reader devices 120 can communicate with a sensor control device 120, but cannot communicate with a remote application server 150 directly. Instead, the reader device 120 is connected to a user device 140 through a wired connection and the analyte data is uploaded by the user device 140 to the remote application server. For many patients, data receiving devices are the primary method of monitoring their analyte levels on a day-to-day basis.

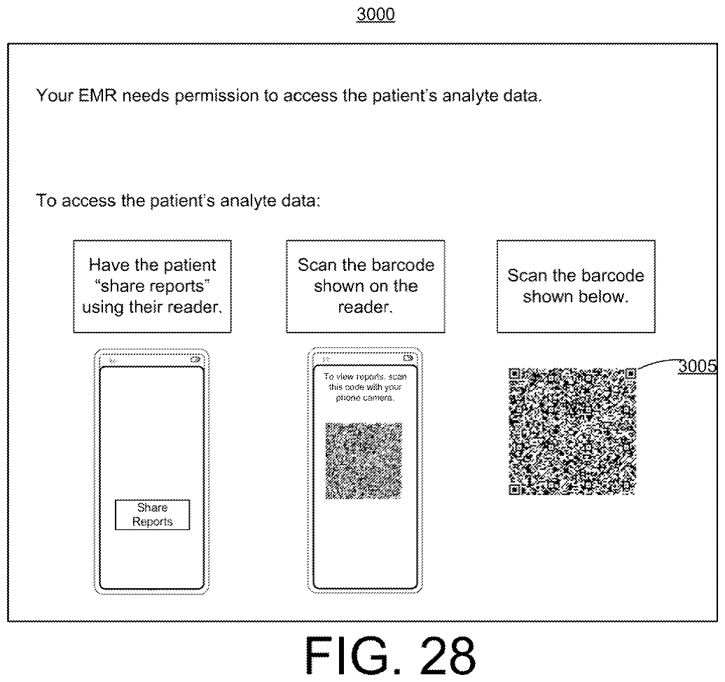

In particular embodiments, even the reader device 120 can be used to grant access to allow a EMR system 1865 to access analyte data stored by the reader device 120 or continuously uploaded to the remote application server 150. The procedure described above, e.g., with respect to at least FIG. 28 can be modified to use the data receiving device.

As an example, when the HCP uses the user device 140 to access the analyte data of the patient (e.g., at 2600), the EMR system 1865 can cause an application interface to be displayed on the user device 140 providing instructions to enable access. The application interface can also include a user input field to enter an access code (such as in the interface 2700 of FIG. 25). The instructions can instruct the HCP to ask the patient to use their reader device 120 to activate a “share reports” feature. This share reports feature can be similar to the share reports feature using reader devices 120 discussed above (e.g., with respect to FIGS. 20-21). The share reports feature causes the reader device 120 to generate a matrix barcode encoded with a URL and encoded analyte data as parameters for the URL. The instructions can further instruct the HCP or a patient to scan the matrix barcode using a suitable application (e.g., a camera application executing on a smartphone or other multi-purpose reader device 120). The matrix barcode can be further encoded with access credentials and other user identifying information to facilitate persistent connection between the EMR system 1855 and the remote application server 155.

Upon scanning the matrix barcode, the encoded analyte data is provided to the report generation system 1860, which generates an access code and access URL. The report generation system 1860 provides the access code and access URL to the device that scanned the matrix barcode (e.g., as shown in FIG. 23). The HCP can enter the access code to the user input field presented in the application associated with the EMR system 1865. The EMR system 1865 then relays the access code to the report generation system 1860 (e.g., as in step 2607). Upon validating the access code, the remote application server 155 can grant access to some or all of the analyte data provided by the multi-purpose data receiving device 130 to the report generation system 1860. This access can be limited to one-time access or continual access per system settings or user preferences of the patient. As the patient uploads additional analyte data, the data can also be shared with the EMR system 1865 as described herein without having to validate access on each occurrence.

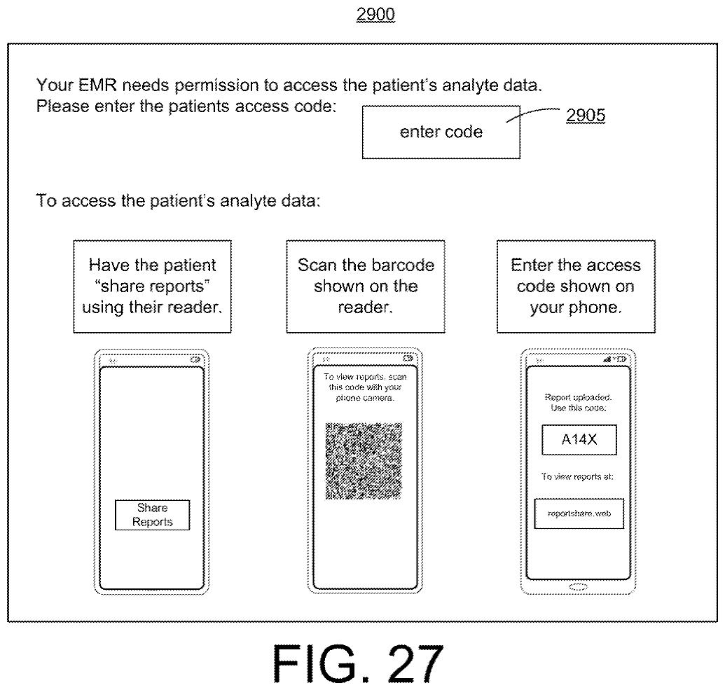

FIG. 27 illustrates an example user interface for granting access according to embodiments herein. In particular, FIG. 27 illustrates an example user interface 2900 that can be used by an application associated with an EMR system 1865 and executing on the user device of an HCP requesting access to a patient's analyte data. The user interface 2900 includes a user input field 2905 for the HCP to enter an access code provided by a patient. The user interface 2900 also includes instructions for the HCP to follow to generate the access code when the patient uses a reader device 120. The instructions, as outlined above, require the HCP to scan a matrix barcode generated by the reader device 120 using another device, such as a smartphone, and enter the access code that will be displayed in the user input field 2905.

This procedure can be further modified to minimize the amount of manual user inputs required to grant authorization. In this embodiment, after the HCP or patient scans the matrix barcode, the URL causes accesses a web application to be displayed on the device that scanned the matrix barcode. The web application includes a camera function and provides instructions for the user to scan an authorization matrix barcode. The authorization matrix barcode is provided in the instructions shown by application associated with the EMR system 1865 and executing on the user device after the HCP has requested access. The authorization matrix barcode includes the information needed for the web application executing on the other device to send the analyte data, reports, or data access credentials to the report generation system 1860 or the remote application server 155 to facilitate access.

FIG. 28 illustrates an example user interface for granting access according to embodiments herein. In particular, FIG. 28 illustrates an example user interface 3000 that can be used by an application associated with an EMR system 1865 and executing on the user device of an HCP requesting access to a patient's analyte data. The user interface 3000 includes instructions for the HCP to follow to generate the access code when the patient uses a reader device 120. The instructions, as outlined above, require the HCP to scan a matrix barcode generated by the reader device 120 using another device, such as a smartphone, and then scan a second matrix barcode 3005 provided in the user interface 3000.

HCP can access data retrieved from and based on a sensor control device 102 in web- or server-based applications. For example, a patient can transfer data from a sensor control device 102 to a reader device 120 or multi-purpose device 130. The data can then be relayed to a remote application server 155 to be processed. In addition to providing standardized reports based on the analyte data, applications executing on or made available by the remote application server 155 can provide guided interpretation of the data. For example, where the analyte of interest includes or is derived from glucose levels, the application executing on the remote application server 155 can provide interpretation of metrics such as an ambulatory glucose profile (AGP) by identifying glucose patterns or areas that need attention. The guided interpretation can include details such as therapy and lifestyle guidance.

Therapy and lifestyle guidance can include assessment of the effect of a patient's current therapy on their health goals, such as their glycemic control. However, this guidance requires knowledge of the patient's current therapy which is not always readily available to the remote application server 155. For example, the remote application server 155 can be operated by the provider of the analyte monitoring system 100, while the information about a patient's current therapy can be housed by a third party (e.g., an HCP system). It would be beneficial to provide for integration of information such as a patient's current therapy within the tools provided by the remote application server 155 to provide, to the HCP and to the patient, more effective therapy and lifestyle guidance, and to provide a comprehensive view of the patient's health and health management.

The HCP can enter therapy information directly into the application executing on the remote application server 155. For example, the HCP can be asked to enter information such as medication names, doses, frequencies, and schedule. Taking, again, glucose management as one example, if the therapy includes insulin, then the HCP can be asked to enter context-specific information for the insulin regimen which can include dose amount, schedule, insulin to carbohydrate ratio, correction factor, and target glucose. If the patient is on insulin pump therapy, then the HCP can enter the basal rates and schedule.

In another embodiment, the HCP can enter the therapy information into their electronic medical records (EMR) system 1865. The therapy information can be retrieved from the EMR system 1865 and imported into the remote application server 155, or can otherwise be accessed on demand by the remote application server 155. In particular, aspects of the remote application server 155 can be integrated with the EMR system 1865.

In some embodiments, therapy information stored by the EMR system 1865 can be stored in a well-structured format. As an example, the therapy information can be stored in a database or table with labeled headers and consistent use and entry enforcement provisions. In such embodiments, therapy information can be extracted from the EMR system 1865 after a mapping between fields of the EMR system 1865 databases and the remote application server 155.

In some embodiments, the therapy information stored by the EMR system 1865 can be included in the form of structure-free data or free text notes. Because there is no straightforward mapping available, additional processing on the therapy information can be required to identify the relevant information and determine how to use the therapy information in the context of the remote application server 155. As an example, free text can be interpreted using keyword association, natural language processing, or pattern matching techniques to extract the relevant information for addition to the remote application server 155.

To encourage the use of well-structured formats, the remote application server 155 can provide a structured data entry interface. As an example, the remote application server 155 can provide an insulin regimen table with dose amount, schedule, insulin to carbohydrate ratio, correction factor, and target glucose, or a table to enter insulin pump therapy parameters. Similar interface options can be provided for other analytes of interest. Once the data is entered into the remote application server 155, the remote application server 155 can use its integration with the EMR system 1865 to convert the data into a format usable by the EMR system 1865. For example, a reverse mapping to the EMR system 1865 can be determined. Alternatively, the structured data from the remote application server 155 can be converted into a format that can be added to the free text fields provided by the EMR system 1865. This will encourage data to be saved in a structured format, even in the free text fields of the EMR system 1865, so that the data can be easily extracted during subsequent imports.

Embodiments disclosed herein include:

F. An analyte monitoring system comprising: one or more processors; and a memory communicatively coupled with the one or more processors comprising instructions that, when executed by the one or more processors, are configured to cause the one or more processors to perform operations includes receiving, from a user of the analyte monitoring system, a request to transfer analyte data associated with the user to another computing device, encoding the analyte data, generating a matrix barcode comprising a uniform resource locator (URL) and the encoded analyte data, wherein the analyte data is formatted as parameters to be passed with the URL by a web browser, and causing the matrix barcode to be presented on a display of the analyte monitoring system.

G. An analyte monitoring system comprising: one or more processors; and a memory communicatively coupled with the one or more processors comprising instructions that, when executed by the one or more processors, are configured to cause the one or more processors to perform operations includes causing a matrix barcode to be scanned via a camera communicatively coupled to the analyte monitoring system, decoding the matrix barcode to obtain a uniform resource locator (URL) and one or more parameters, wherein the one or more parameters comprise at least analyte data, presenting, using the URL and the one or more parameters, a web request to a server associated with the URL, receiving, by the analyte monitoring system and from the server associated with the URL, a report based on the analyte data included in the parameters, wherein the report was generated by the server based on the parameters presented with the URL, and causing the report to be output via a display.

H. A server computing device of an analyte monitoring system comprising: one or more processors; and a memory communicatively coupled with the one or more processors comprising instructions that, when executed by the one or more processors, are configured to cause the one or more processors to perform operations includes receiving a first web request comprising one or more parameters, wherein the one or more parameters comprise analyte data associated with a user of the analyte monitoring system, generating a report based on the received analyte data, associating an access code with the report, receiving a second web request comprising one or more parameters, wherein the one or more parameters comprise the access code, retrieving the report based on the access code, and causing the report to be output on a display.

I. An analyte monitoring system comprising: one or more processors; and a memory communicatively coupled with the one or more processors comprising instructions that, when executed by the one or more processors, are configured to cause the one or more processors to perform operations includes establishing a communication session between the analyte monitoring system and an electronic medical records (EMR) system, identifying therapy information associated with a user and stored in the EMR system, causing the therapy information associated with the user to be transferred from the EMR system to the application server, converting the therapy information for use by the analyte monitoring system, storing the converted therapy information in a database accessible to the analyte monitoring system, causing the therapy information to be displayed for a user of the analyte monitoring system.

J. An analyte monitoring system comprising: one or more processors; and a memory communicatively coupled with the one or more processors comprising instructions that, when executed by the one or more processors, are configured to cause the one or more processors to perform operations includes establishing a communication session between the analyte monitoring system and an electronic medical records (EMR) system, receiving a request from a user device for the EMR system to access analyte data associated with a patient and stored in a database accessible to the analyte monitoring system, causing a data receiving device associated with the analyte monitoring system and the patient to display an access code associated with the analyte data associated with the patient, receiving a second access code from the user device, comparing the access code displayed by the data receiving device and the second access code, and in response to determining that the access code displayed by the data receiving device and the second access code are equivalent, granting access to the EMR system to access the analyte data associated with the patient.

Each of embodiments, F, G, H, I and J may have one or more of the following additional elements in any combination: Element 1: wherein the instructions are further configured to cause the one or more processors to perform further operations comprising selecting the analyte data for encoding based on the analyte data corresponding to a predetermined range of time. Element 2: wherein the instructions to encode the analyte data are further configured to cause the one or more processors to perform further operations includes calculating one or more derivative values from the analyte data, and encoding the derivative values with the analyte data. Element 3: wherein encoding the analyte data comprises reducing a level of precision associated with the analyte data, wherein the level of precision is selected based on an actual value of the analyte data. Element 4: wherein the first web request is received from a first computing device and the second web request is received form a second computing device. Element 5: wherein the web request and parameters are generated by the first computing device subsequent to scanning a matrix barcode. Element 6: wherein the therapy information is stored a first structured format; and wherein converting the therapy information comprises mapping the first structure format to a second structured format associated with analyte monitoring system. Element 7: wherein the therapy information is stored in a free text format; and wherein converting the therapy information includes interpreting the therapy information using a natural language processing, and storing the information in a structured format associated with the analyte monitoring system. Element 8: wherein the instructions are further configured to cause the one or more processors to perform further operations includes receiving additional therapy information through a structured format input, converting the additional therapy information for use by the EMR system, and transferring the converted additional therapy information to the EMR system for storage. Element 9: wherein the access code is generated by the data receiving device and the analyte monitoring system receives the access code from the data receiving device. Element 10: wherein the access code is generated by the analyte monitoring system and the data receiving device receives the access code from the analyte monitoring system. Element 11: wherein the instructions are further configured to cause the one or more processors to perform further operations including cause the data receiving device to display an authorization request, wherein the EMR system is granted access in response to an affirmative response to the authorization request. Element 12: wherein one or more of the access code and the second access code comprise a matrix barcode.

### Example Embodiments of Sensor Results Interfaces

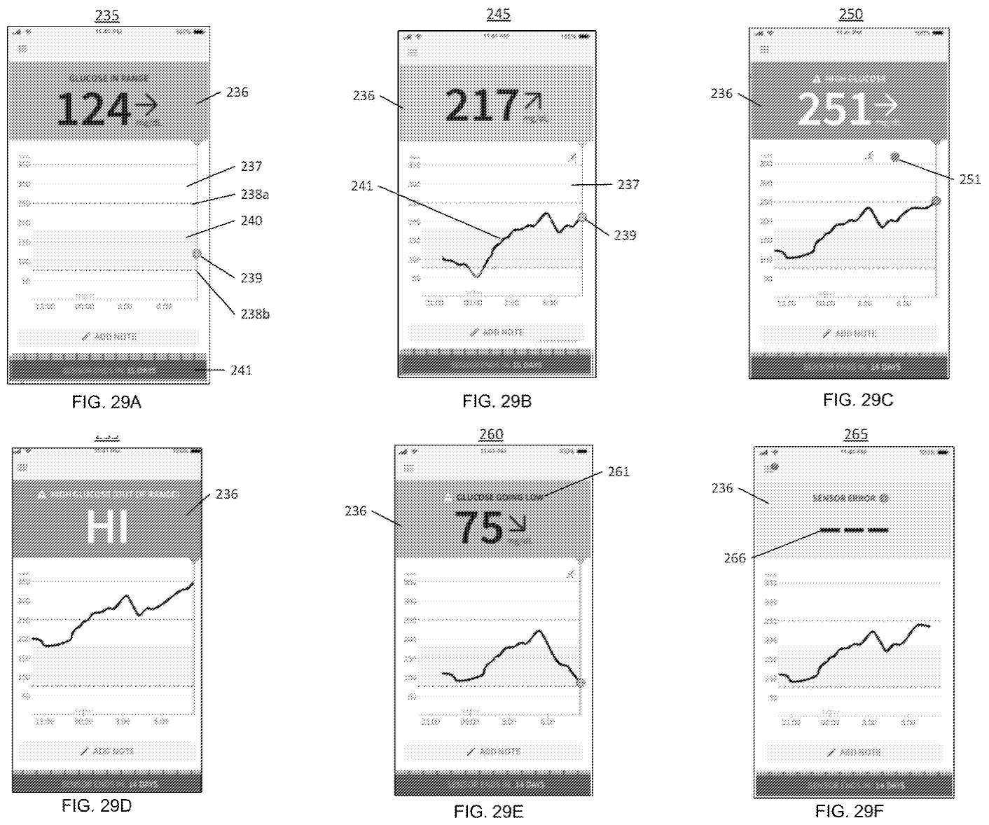

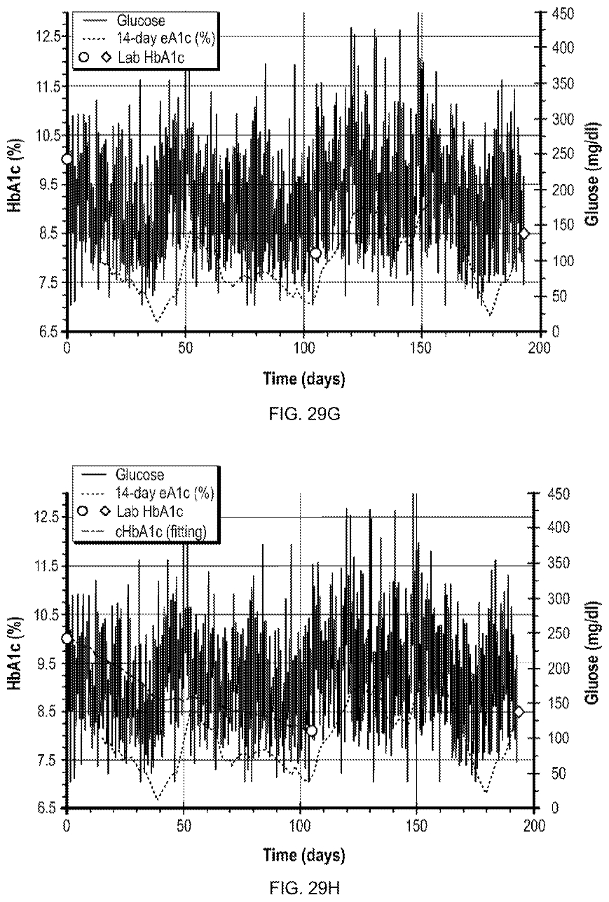

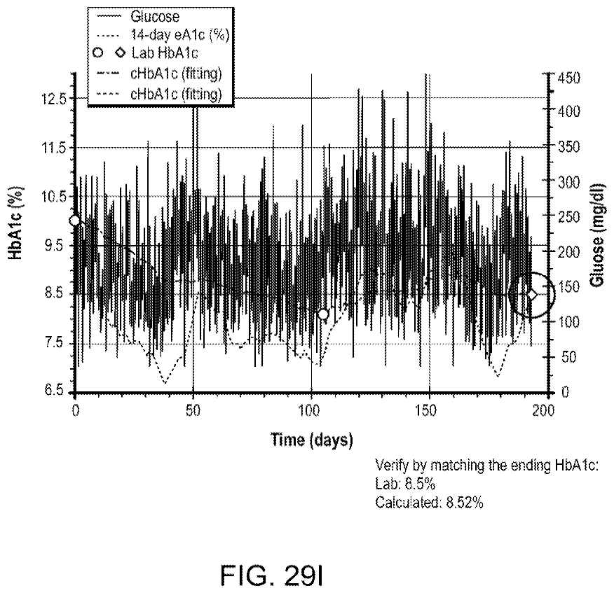

FIGS. 29A to 29F depict example embodiments of sensor results interfaces or GUIs for analyte monitoring systems. In accordance with the disclosed subject matter, the sensor results GUIs described herein are configured to display analyte data and other health information through a user interface application (e.g., software) installed on a reader device, such as a smart phone or a receiver, like those described with respect to FIG. 2B. Those of skill in the art will also appreciate that a user interface application with a sensor results interface or GUI can also be implemented on a local computer system or other computing device (e.g., wearable computing devices, smart watches, tablet computer, etc.).

Referring first to FIG. 29A, sensor results GUI 235 depicts an interface comprising a first portion 236 that can include a numeric representation of a current analyte concentration value (e.g., a current glucose value), a directional arrow to indicate an analyte trend direction, and a text description to provide contextual information such as, for example, whether the user's analyte level is in range (e.g., “Glucose in Range”). According to embodiments, first portion 236 can include a numeric representation of a personalized analyte concentration value (e.g., a personalized glucose value), as determined using a kinetic model as disclosed herein below. First portion 236 can also comprise a color or shade that is indicative of an analyte concentration or trend. For example, as shown in FIG. 29A, first portion 236 is a green shade, indicating that the user's analyte level (for example, not limitation, current or personalized glucose level) is within a target range. According to some embodiments, for example, a red shade can indicate an analyte level below a low analyte level threshold, an orange shade can indicate an analyte level above a high analyte level threshold, and an yellow shade can indicate an analyte level outside a target range. According to embodiments, the target range can be a personalized target glucose range as determined using a kinetic model as disclosed herein below.

In addition, according to some embodiments, sensor results GUI 235 also includes a second portion 237 comprising a graphical representation of analyte data. In particular, second portion 237 includes an analyte trend graph reflecting an analyte concentration, as shown by the y-axis, over a predetermined time period, as shown by the x-axis. According to embodiments, second portion 237 can include a personalized analyte trend graph reflecting a personalized analyte concentration, as determined using a kinetic model as disclosed herein below, as shown by the y-axis, over a predetermined time period, as shown by the x-axis. In some embodiments, the predetermined time period can be shown in five-minute increments, with a total of twelve hours of data. Those of skill in the art will appreciate, however, that other time increments and durations of analyte data can be utilized and are fully within the scope of this disclosure. Second portion 237 can also include a point 239 on the analyte trend graph to indicate the current analyte concentration value, a shaded green area 240 to indicate a target analyte range, and two dotted lines 238a and 238b to indicate, respectively, a high analyte threshold and a low analyte threshold. According to embodiments, point 239 on a personalized analyte trend graph can indicate the current personalized concentration value, shaded green area 240 to indicate a personalized target analyte range, and/or two dotted lines 238a and 238b to indicate, respectively, a personalized high analyte threshold and a personalized low analyte threshold. According to some embodiments, GUI 235 can also include a third portion 241 comprising a graphical indicator and textual information representative of a remaining amount of sensor life.

Referring next to FIG. 29B, another example embodiment of a sensor results GUI 245 is depicted. In accordance with the disclosed subject matter, first portion 236 is shown in a yellow shade to indicate that the user's current analyte concentration is not within a target range. According to embodiments, the currently analyte concentration can include a current personalized analyte concentration, and/or target range can be a personalized target range, as determined using a kinetic model as described herein. In addition, second portion 237 includes: an analyte trend line 241 which can reflect historical analyte levels over time and a current analyte data point 239 to indicate the current analyte concentration value (shown in yellow to indicate that the current value is outside the target range). According to embodiments, analyte trend line 241 can include historical personalized analyte levels over a time current analyte data point 239 can indicate personalized analyte concentration value.

According to another aspect of the embodiments, data on sensor results GUI 245 is automatically updated or refreshed according to an update interval (e.g., every second, every minute, every 5 minutes, etc.). For example, according to many of the embodiments, as analyte data is received by the reader device, sensor results GUI 245 will update: (1) the current analyte concentration value shown in first portion 236, and (2) the analyte trend line 241 and current analyte data point 239 show in second portion 237. Furthermore, in some embodiments, the automatically updating analyte data can cause older historical analyte data (e.g., in the left portion of analyte trend line 241) to no longer be displayed. According to embodiments, current analyte concentration value can include current personalized current value, analyte trend line 241 can include personalized analyte trend line 241, and current analyte data point 239 can include a current personalized analyte data point 239.

FIG. 29C is another example embodiment of a sensor results GUI 250. According to the depicted embodiment, sensor results GUI 250 includes first portion 236 which is shown in an orange shade to indicate that the user's analyte levels are above a high glucose threshold (e.g., greater than 250 mg/dL). According to embodiments, the user's analyte levels shown can include a current personalized analyte concentration, and high glucose threshold can include a personalized high glucose threshold. Sensor results GUI 250 also depicts health information icons 251, such as an exercise icon or an apple icon, to reflect user logged entries indicating the times when the user had exercised or eaten a meal.

FIG. 29D is another example embodiment of a sensor results GUI 255. According to the depicted embodiments, sensor results GUI 255 includes first portion 236 which is also shown in an orange shade to indicate that the user's analyte levels are above a high glucose threshold. As discussed above, according to embodiments, user's analyte levels shown can include a current personalized analyte concentration, and high glucose threshold can include a personalized high glucose threshold. As can be seen in FIG. 29D, first portion 236 does not report a numeric value but instead displays the text “HI” to indicate that the current analyte concentration value is outside a glucose reporting range high limit. Although not depicted in FIG. 29D, those of skill in the art will understand that, conversely, an analyte concentration below a glucose reporting range low limit will cause first portion 236 not to display a numeric value, but instead, the text “LO”. According to embodiments, first portion 236 can display the text “HI” to indicate that the personalized analyte concentration value is outside a personalized glucose reporting range high limit, and, conversely, first portion 236 would display “LO” when a personalized analyte concentration is below a glucose reporting range low limit.

FIG. 29E is another example embodiment of a sensor results GUI 260. According to the depicted embodiments, sensor results GUI 260 includes first portion 236 which is shown in a green shade to indicate that the user's current analyte level is within the target range. According to embodiments, user's current analyte levels can include a current personalized analyte level, and the target range can include a personalized target range. In addition, according to the depicted embodiments, first portion 236 of GUI 260 includes the text, “GLUCOSE GOING LOW,” which can indicate to the user that his or her analyte concentration value is predicted to drop below a predicted low analyte level threshold within a predetermined amount of time (e.g., predicted glucose will fall below 75 mg/dL within 15 minutes). Those of skill in the art will understand that if a user's analyte level is predicted to rise above a predicted high analyte level threshold within a predetermined amount of time, sensor results GUI 260 can display a “GLUCOSE GOING HIGH” message. According to embodiments, analyte concentration value can include a personalized analyte concentration value, and predicted low analyte level and predicted high analyte level can include a predicted personalized low analyte level and a predicted high analyte level, respectively.

FIG. 29F is another example embodiment of a sensor results GUI 265. According to the depicted embodiments, sensor results GUI 265 depicts first portion 236 when there is a sensor error. In accordance with the disclosed subject matter, first portion 236 includes three dashed lines 266 in place of the current analyte concentration value to indicate that a current analyte value is not available. According to embodiments, current analyte concentration value can include a current personalized analyte concentration value. In some embodiments, three dashed lines 266 can indicate one or more error conditions such as, for example, (1) a no signal condition; (2) a signal loss condition; (3) sensor too hot/cold condition; or (4) a glucose level unavailable condition. Furthermore, as can be seen in FIG. 29F, first portion 236 comprises a gray shading (instead of green, yellow, orange, or red) to indicate that no current analyte data (or current personalized analyte data) is available. In addition, according to another aspect of the embodiments, second portion 237 can be configured to display the historical analyte data in the analyte trend graph, even though there is an error condition preventing the display of a numeric value for a current analyte concentration in first portion 236. According to embodiments, historical analyte data can include historical personalized analyte data. However, as shown in FIG. 29F, no current analyte concentration value data point is shown on the analyte trend graph of second portion 237.

FIG. 29G is a glucose monitoring data interface which includes a graphical representation of the glucose monitoring data (right y-axis) for 200 days, superimposed with three laboratory HbA1c values (left y-axis) and the estimated HbA1c values (left y-axis) based on the 14-day eHbA1c model as disclosed in International Publication No. WO2021/108419 to Xu and WO2020/086934 to Xu, which are incorporated by reference in its entirety herein. As illustrated, the estimated HbA1c derived from the 14-day HbA1c model has very dramatic changes over time. However, it is unlikely that HbA1c can change this fast.

FIG. 29H is a glucose monitoring data interface which includes the graphical representation of FIG. 29G superimposed with a calculated HbA1c (left y-axis) for the first 100 days determined using k_{gly} and k_{age} per the methods described in International Publication No. WO2021/108419 and WO2020/086934 to Xu, which are incorporated by reference in its entirety herein.

FIG. 29I is a glucose monitoring data interface which includes the graphical representation of FIG. 29H superimposed with the calculated HbA1c (extension from day 100 to day 200, left y-axis) for the following 100 days using the k_{gly} and k_{age} determined relative to FIG. 29H per the methods described in International Publication No. WO2021/108419 and WO2020/086934 to Xu, which are incorporated by reference in its entirety herein. The third HbA1c value was not considered in this method, but the model described, predicted the measured value of the third HbA1c value, which illustrates that the model described herein is in close agreement with reality.

### Example Embodiments of Time-In-Ranges Interfaces

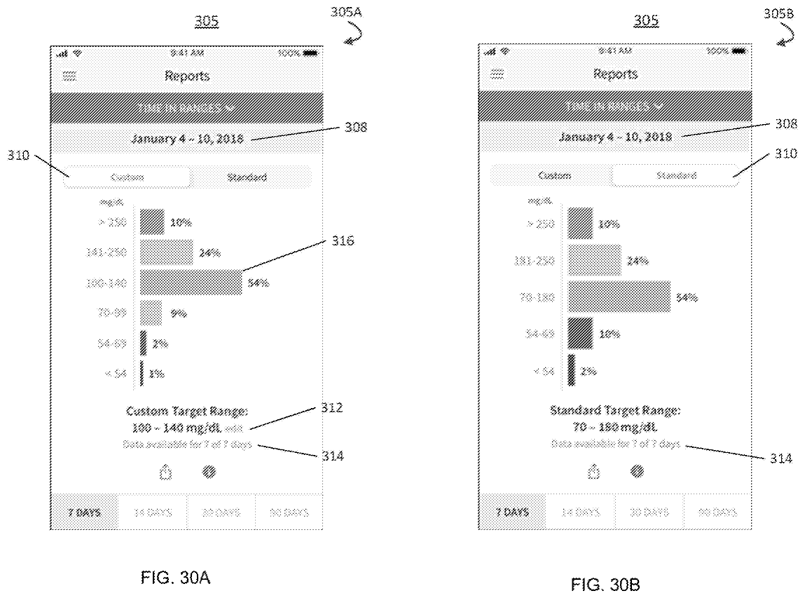

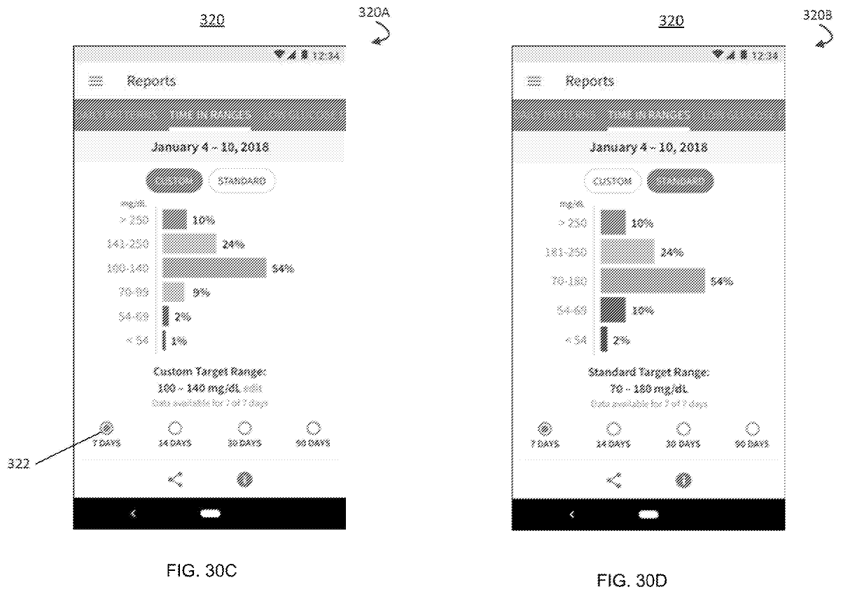

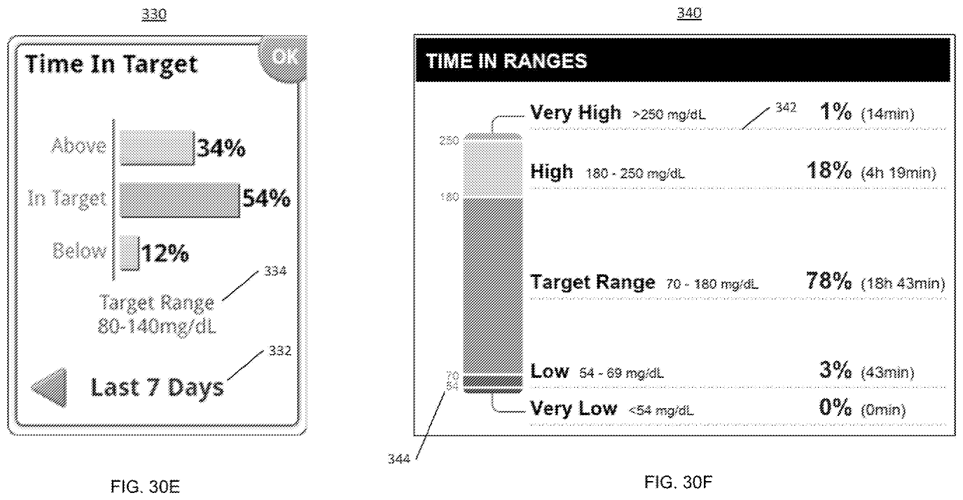

FIGS. 30A to 30F depict example embodiments of GUIs for analyte monitoring systems. In particular, FIGS. 30A to 30F depict Time-in-Ranges (also referred to as Time-in-Range and/or Time-in-Target) GUIs, each of which comprise a plurality of bars or bar portions, wherein each bar or bar portion indicates an amount of time that a user's analyte level is within a predefined analyte range correlating with the bar or bar portion. In some embodiments, for example, the amount of time can be expressed as a percentage of a predefined amount of time. According to embodiments, FIGS. 30A to 30F, as described below, can also depict personalized Time-in-Ranges (also referred to as personalized Time-in-Target) GUIs, each of which comprise a plurality of bars or bar portions, wherein each bar or bar portion indicates an amount of time that a user's personalized analyte level is within a predefined personalized analyte range correlating with the bar or bar portion.

Turning to FIGS. 30A and 30B, an example embodiment of a Time-in-Ranges GUI 305 is shown, wherein Time-in-Ranges GUI 305 comprises a “Custom” Time-in-Ranges view 305A and a “Standard” Time-in-Ranges view 305B, with a slidable element 310 that allows the user to select between the two views. In accordance with the disclosed subject matter, Time-in-Ranges views 305A, 305B can each comprise multiple bars, wherein each bar indicates an amount of time that a user's analyte level is within a predefined analyte range correlating with the bar. According to embodiments, user's analyte level can include personalized analyte level. In some embodiments, Time-in-Ranges views 305A, 305B further comprise a date range indicator 308, showing relevant dates associated with the displayed plurality of bars, and a data availability indicator 314, showing the period(s) of time in which analyte data is available for the displayed analyte data (e.g., “Data available for 7 of 7 days”).

Referring to FIG. 30A, “Custom” Time-in-Ranges view 305A includes six bars comprising (from top to bottom): a first bar indicating that the user's glucose range is above 250 mg/dL for 10% of a predefined amount of time, a second bar indicating that the user's glucose range is between 141 and 250 mg/dL for 24% of the predefined amount of time, a third bar 316 indicating that the user's glucose range is between 100 and 140 mg/dL for 54% of the predefined amount of time, a fourth bar indicating that the user's glucose range is between 70 and 99 mg/dL for 9% of the predefined amount of time, a fifth bar indicating that the user's glucose range is between 54 and 69 mg/dL for 2% of the predefined amount of time, and a sixth bar indicating that the user's glucose range is less than 54 mg/dL for 1% of the predefined amount of time. Those of skill in the art will recognize that the glucose ranges and percentages of time associated with each bar can vary depending on the ranges defined by the user and the available analyte data of the user, and that user's glucose range can include user's personalized glucose range. Furthermore, although FIGS. 30A and 3B show a predefined amount of time 314 equal to seven days, those of skill in the art will appreciate that other predefined amounts of time can be utilized (e.g., one day, three days, fourteen days, thirty days, ninety days, etc.), and are fully within the scope of this disclosure.

According to another aspect of the embodiments, “Custom” Time-in-Ranges view 305A also includes a user-definable custom target range 312 that includes an actionable “edit” link that allows a user to define and/or change the custom target range. As shown in “Custom” Time-in-Ranges view 305A, the custom target range 312 has been defined as a glucose range between 100 and 140 mg/dL and corresponds with third bar 316 of the plurality of bars. Those of skill in the art will also appreciate that, in other embodiments, more than one range can be adjustable by the user, and such embodiments are fully within the scope of this disclosure. According to embodiments, custom target range 312 can include custom personalized target ranges.

Referring to FIG. 30B, “Standard” Time-in-Ranges view 305B includes five bars comprising (from top to bottom): a first bar indicating that the user's glucose range is above 250 mg/dL for 10% of a predefined amount of time, a second bar indicating that the user's glucose range is between 181 and 250 mg/dL for 24% of the predefined amount of time, a third bar indicating that the user's glucose range is between 70 and 180 mg/dL for 54% of the predefined amount of time, a fourth bar indicating that the user's glucose range is between 54 and 69 mg/dL for 10% of the predefined amount of time, and a fifth bar indicating that the user's glucose range is less than 54 mg/dL for 2% of the predefined amount of time. As with the “Custom” Time-in-Ranges view 305A, those of skill in the art will recognize that the percentages of time associated with each bar can vary depending on the available analyte data of the user. Additionally, according to embodiments, the user's glucose range can include user's personalized glucose range, and the numerical glucose ranges associated with the five bars can be adjusted for a user's personalized glucose range. For example, not limitation, personalized glucose ranges can for each of the five bars can be calculated using the models as disclosed herein below. Unlike the “Custom” Time-in-Ranges view 305A, however, the glucose ranges shown in “Standard” view 305B cannot be adjusted by the user.

FIGS. 30C and 30D depict another example embodiment of Time-in-Ranges GUI 320 with multiple views, 320A and 320B, which are analogous to the views shown in FIGS. 30A and 30B, respectively. According to some embodiments, Time-in-Ranges GUI 320 can further include one or more selectable icons 322 (e.g., radio button, check box, slider, switch, etc.) that allow a user to select a predefined amount of time over which the user's analyte data will be shown in the Time-in-Range GUI 320. For example, as shown in FIGS. 30C and 30D, selectable icons 322 can be used to select a predefined amount of time of seven days, fourteen days, thirty days, or ninety days. Those of skill in the art will appreciate that other predefined amounts of time can be utilized and are fully within the scope of this disclosure.

FIG. 30E depicts an example embodiment of a Time-in-Target GUI 330, which can be visually output to a display of a reader device (e.g., a dedicated reader device, a meter device, etc.). In accordance with the disclosed subject matter, Time-in-Target GUI 330 includes three bars comprising (from top to bottom): a first bar indicating that the user's glucose range is above a predefined target range for 34% of a predefined amount of time, a second bar indicating that the user's glucose range is within the predefined target range for 54% of the predefined amount of time, and a third bar indicating that the user's glucose range is below the predefined target range for 12% of the predefined amount of time. Those of skill in the art will recognize that the percentages of time associated with each bar can vary depending on the available analyte data of the user, the user's glucose range can include user's personalized glucose range. Furthermore, although FIG. 30E shows a predefined amount of time 332 equal to the last seven days and a predefined target range 334 of 80 to 140 mg/dL, those of skill in the art will appreciate that other predefined amounts of time (e.g., one day, three days, fourteen days, thirty days, ninety days, etc.) and/or predefined target ranges (e.g., 70 to 180 mg/dL) can be utilized, and are fully within the scope of this disclosure. According to embodiments, predefined target range can be a predefined personalized target range determined using a kinetic model as disclosed herein.

FIG. 30F depicts another example embodiment of a Time-in-Ranges GUI 340, which includes a single bar comprising five bar portions including (from top to bottom): a first bar portion indicating that the user's glucose range is “Very High” or above 250 mg/dL for 1% (14 minutes) of a predefined amount of time, a second bar portion indicating that the user's glucose range is “High” or between 180 and 250 mg/dL for 18% (4 hours and 19 minutes) of the predefined amount of time, a third bar portion indicating that the user's glucose range is within a “Target Range” or between 70 and 180 mg/dL for 78% (18 hours and 43 minutes) of the predefined amount of time, a fourth bar portion indicating that the user's glucose range is “Low” or between 54 and 69 mg/dL for 3% (43 minutes) of the predefined amount of time, and a fifth bar portion indicating that the user's glucose range is “Very Low” or less than 54 mg/dL for 0% (0 minutes) of the predefined amount of time. As shown in FIG. 30F, according to some embodiments, Time-in-Ranges GUI 340 can display text adjacent to each bar portion indicating an actual amount of time, e.g., in hours and/or minutes. According to embodiments, the numerical values associated with the five bars can be adjusted for a user's personalized glucose target range.

According to one aspect of the embodiment shown in FIG. 30F, each bar portion of Time-in-Ranges GUI 340 can comprise a different color. In some embodiments, bar portions can be separated by dashed or dotted lines 342 and/or interlineated with numeric markers 344 to indicate the ranges reflected by the adjacent bar portions. In some embodiments, the time in ranges reflected by the bar portions can be further expressed as a percentage, an actual amount of time (e.g., 4 hours and 19 minutes), or, as shown in FIG. 30F, both. Furthermore, those of skill in the art will recognize that the percentages of time associated with each bar portion can vary depending on the analyte data of the user. In some embodiments of Time-in-Ranges GUI 340, the Target Range can be configured by the user. In other embodiments, the Target Range of Time-in-Ranges GUI 340 is not modifiable by the user.

### Example Embodiments of Analyte Level and Trend Alert Interfaces

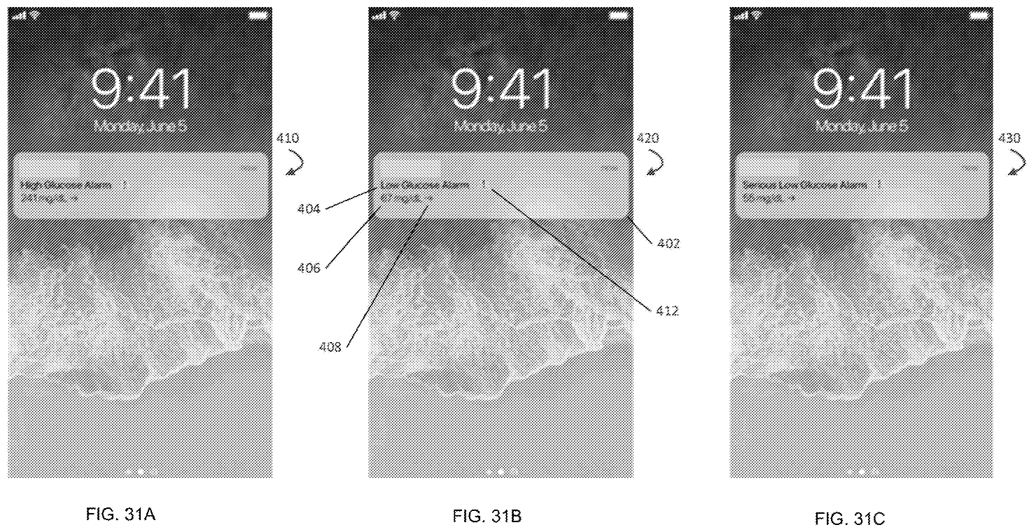

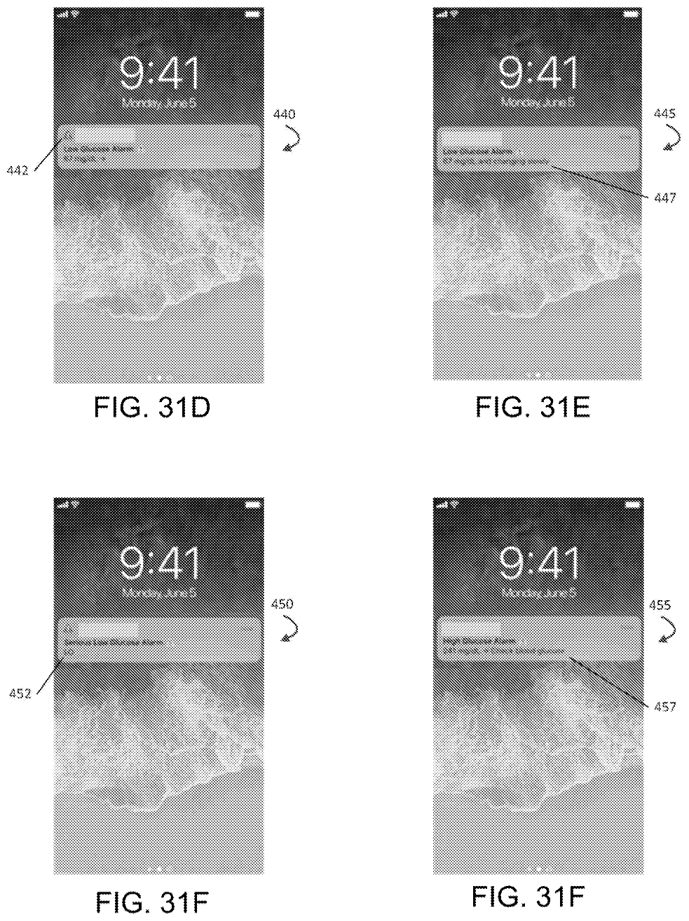

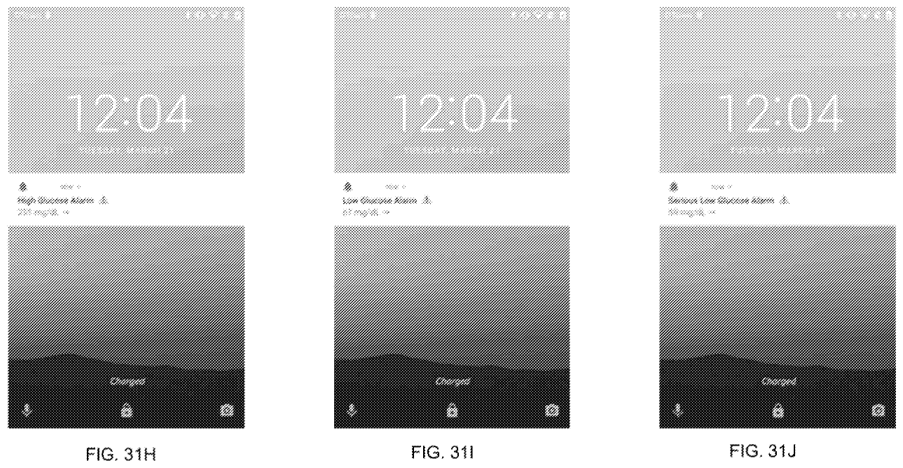

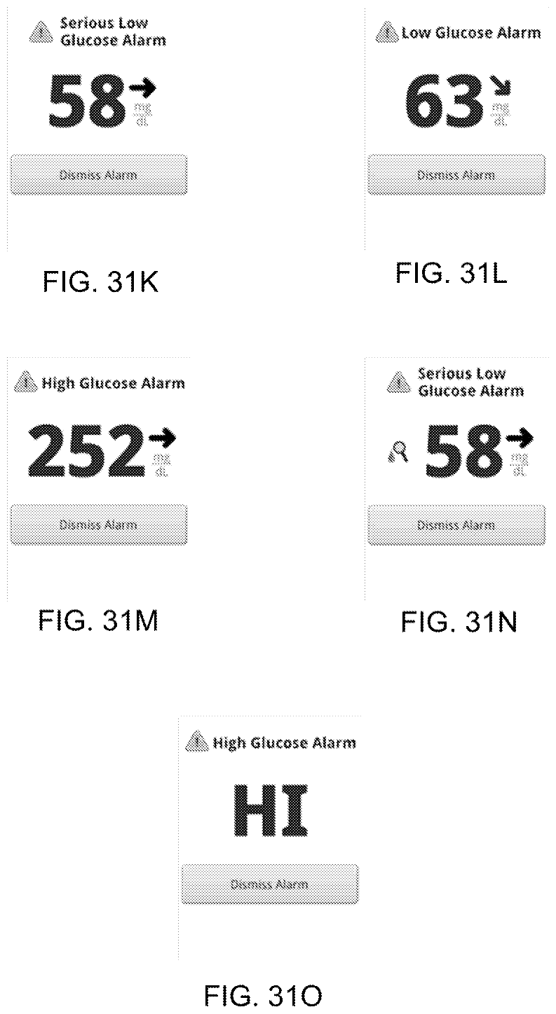

FIGS. 31A to 310 depict example embodiments of Analyte Level/Trend Alert GUIs for analyte monitoring systems. In accordance with the disclosed subject matter, the Analyte Level/Trend Alert GUIs comprise an audio or a visual notification (e.g., prompt, alert, alarm, pop-up window, banner notification, etc.), wherein the visual notification includes an alarm condition, an analyte level measurement associated with the alarm condition, and a trend indicator associated with the alarm condition. According to embodiment, at least one processor is configured to output a notification if at least one of the plurality of personalized glucose metrics is at or above the corresponding plurality of personalized glucose target. Notification can include an audio or a visual notification (e.g., prompt, alert, alarm, pop-up window, banner notification, etc.).

Turning to FIGS. 31A to 31C, example embodiments of a High Glucose Alarm 410, Low Glucose Alarm 420, and a Serious Low Glucose Alarms 430 are depicted, respectively, wherein each alarm comprises a pop-up window 402 containing an alarm condition text 404 (e.g., “Low Glucose Alarm”), an analyte level measurement 406 (e.g., a current glucose level of 67 mg/dL) associated with the alarm condition, and a trend indicator 408 (e.g., a trend arrow or directional arrow) associated with the alarm condition. In some embodiments, an alarm icon 412 can be adjacent to the alarm condition text 404. According to embodiments, analyte level measurement 406 can include a personalized analyte level measurement (e.g., a current personalized glucose level of 67 mg/dL).

Referring next to FIGS. 31D to 31G, additional example embodiments of Low Glucose Alarms 440, 445, Serious Low Glucose Alarm 450, and High Glucose Alarm 455 are depicted, respectively. As shown in FIG. 31D, Low Glucose Alarm 440 is similar to the Low Glucose Alarm of FIG. 31B (e.g., comprises a pop-up window containing an alarm condition text, an analyte level measurement associated with the alarm condition, and a trend indicator associated with the alarm condition), but further includes an alert icon 442 to indicate that the alarm has been configured as an alert (e.g., will display, play a sound, vibrate, even if the device is locked or if the device's “Do Not Disturb” setting has been enabled). With respect to FIG. 31E, Low Glucose Alarm 445 is also similar to the Low Glucose Alarm of FIG. 31B, but instead of including a trend arrow, Log Glucose Alarm 445 includes a textual trend indicator 447. According to one aspect of some embodiments, textual trend indicator 447 can be enabled through a device's Accessibility settings such that the device will “read” the textual trend indicator 447 to the user via the device's text-to-speech feature (e.g., Voiceover for iOS or Select-to-Speak for Android).

Referring next to FIG. 31F, Low Glucose Alarm 450 is similar to the Low Glucose Alarm of FIG. 31D (including the alert icon), but instead of displaying an analyte level measurement associated with an alarm condition and a trend indicator associated with the alarm condition, Low Glucose Alarm 450 displays an out-of-range indicator 452 to indicate that the current glucose level is either above or below a predetermined reportable analyte level range (e.g., “HI” or “LO”). According to embodiments, the current glucose level can include a current personalized glucose level, and the predetermined reportable analyte level range can include a predetermined reportable personalized analyte level range. With respect to FIG. 31G, High Glucose Alarm 455 is similar to the High Glucose Alarm of FIG. 31A (e.g., comprises a pop-up window containing an alarm condition text, an analyte level measurement associated with the alarm condition, and a trend indicator associated with the alarm condition), but further includes an instruction to the user 457. In some embodiments, for example, the instruction can be a prompt for the user to “Check blood glucose.” Those of skill in the art will appreciate that other instructions or prompts can be implemented (e.g., administer a corrective bolus, eat a meal, etc.).

Furthermore, although FIGS. 31A to 31G depict example embodiments of Analyte Level/Trend Alert GUIs that are displayed on smart phones having an iOS operating system, those of skill in the art will also appreciate that the Analyte Level/Trend Alert GUIs can be implemented on other devices including, e.g., smart phones with other operating systems, smart watches, wearables, reader devices, tablet computing devices, blood glucose meters, laptops, desktops, and workstations, to name a few. FIGS. 31H to 31J, for example, depict example embodiments of a High Glucose Alarm, Low Glucose Alarm, and a Serious Low Glucose Alarm for a smart phone having an Android Operating System. Similarly, FIGS. 31K to 310 depict, respectively, example embodiments of a Serious Low Glucose Alarm, Low Glucose Alarm, High Glucose Alarm, Serious Low Glucose Alarm (with a Check Blood Glucose icon), and High Glucose Alarm (with an out-of-range indicator) for a reader device.

### Example Embodiments of Sensor Usage Interfaces

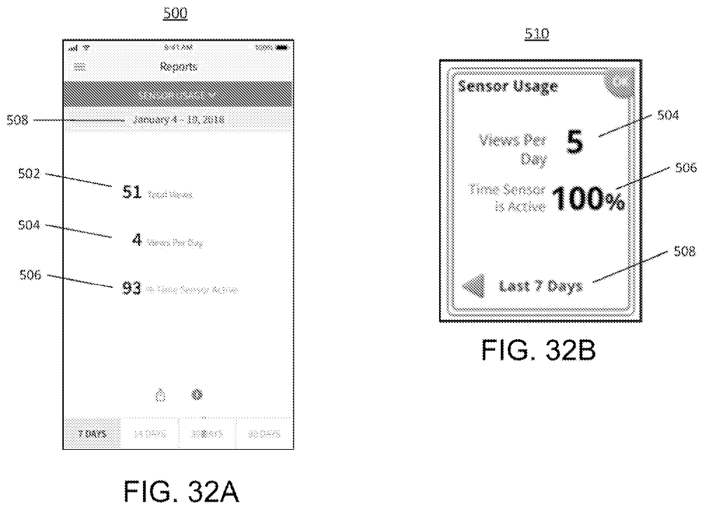

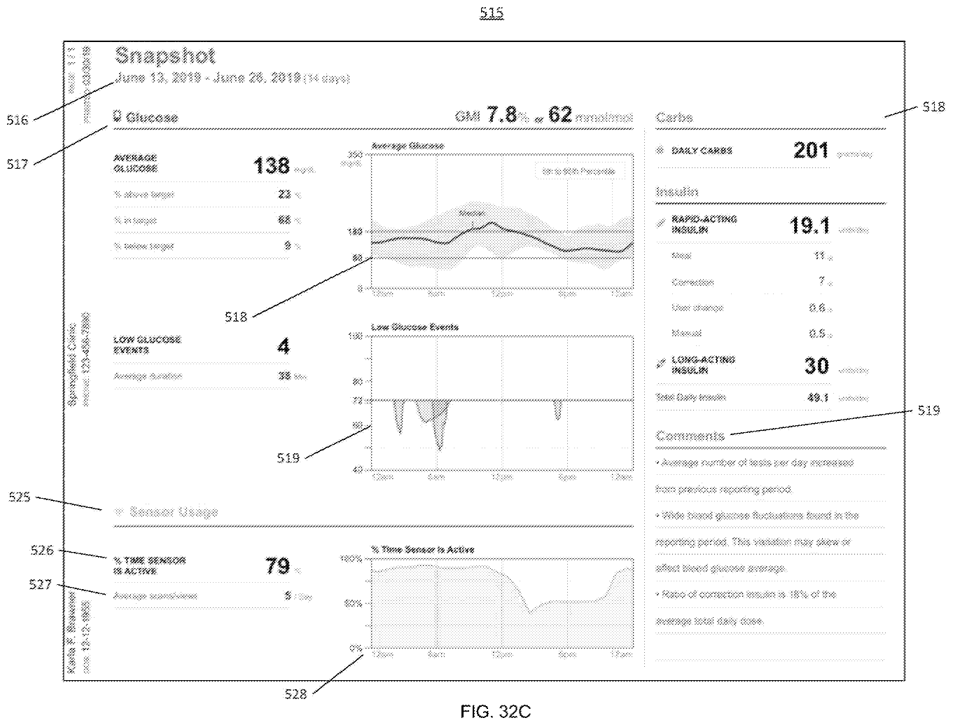

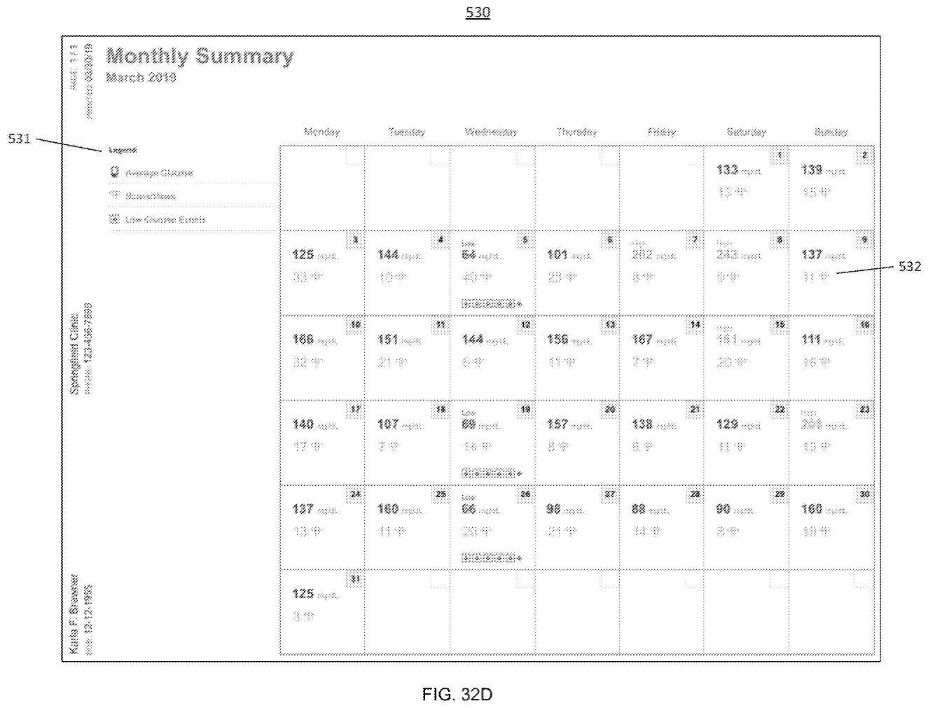

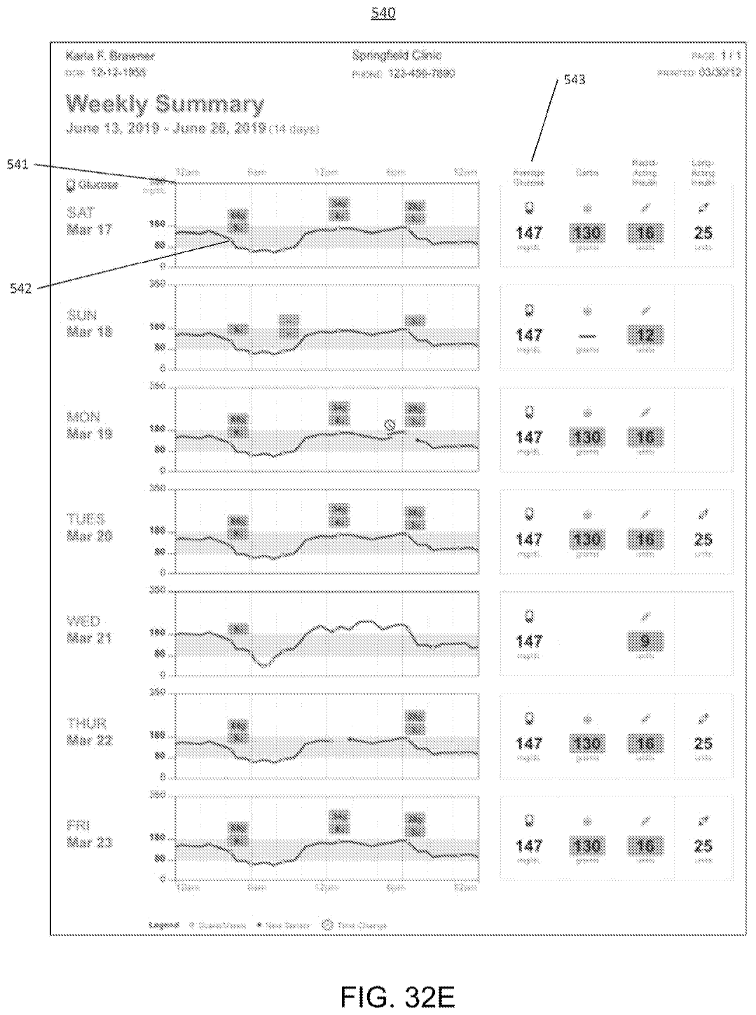

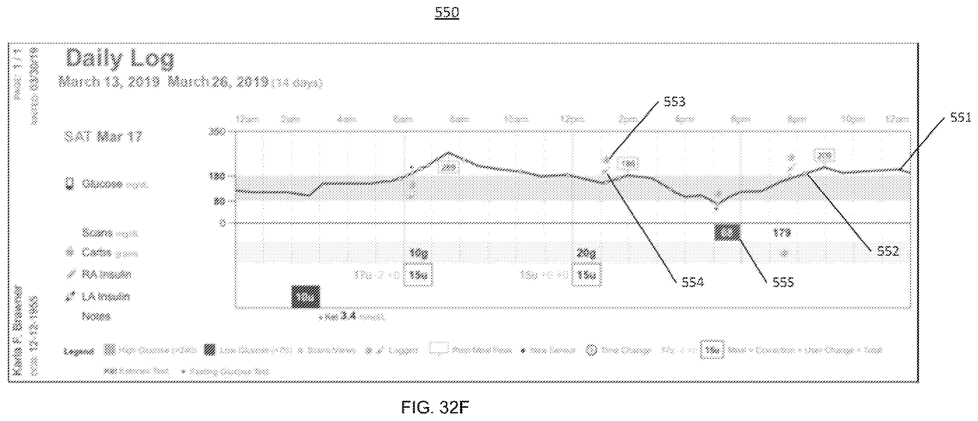

FIGS. 32A to 32F depict example embodiments of sensor usage interfaces relating to GUIs for analyte monitoring systems. In accordance with the disclosed subject matter, sensor usage interfaces provide for technological improvements including the capability to quantify and promote user engagement with analyte monitoring systems. For example, the user can benefit from subtle behavioral modification as the sensor usage interface encourages more frequent interaction with the device and the expected improvement in outcomes. The user can also benefit from increased frequent interaction which leads to improvement in a number of metabolic parameters, as discussed in further detail below.

In some embodiments, HCPs can receive a report of the user's frequency of interaction and a history of the patient's recorded metabolic parameters (e.g., estimated HbA1c levels, time in range of 70-180 mg/dL, etc.). If an HCP sees certain patients in their practice are less engaged than others, the HCPs can focus their efforts on improving engagement in users/patients that are less engaged than others. HCPs can benefit from more cumulative statistics (such as average glucose views per day, average glucose views before/after meals, average glucose views on “in-control” vs. “out-of-control” days or time of day) which may be obtained from the record of user's interaction frequency with the analyte monitoring systems and which can be used to understand why a patient may not be realizing expected gains from the analyte monitoring system. If an HCP sees that a patient is not benefiting as expected from the analyte monitoring system, they may recommend an increased level of interaction (e.g., increase interaction target level). Accordingly, an HCP can change the predetermined target level of interaction.

In some embodiments, caregivers can receive a report of the user's frequency of interaction. In turn, caregivers may be able to nudge the user to improve interaction with the analyte monitoring system. The caregivers may be able to use the data to better understand and improve their level of engagement with the user's analyte monitoring systems or alter therapy decisions.

According to some embodiments, for example, a sensor usage interface can include the visual display of one or more “view” metrics, each of which can be indicative of a measure of user engagement or interaction with the analyte monitoring system. A “view” can comprise, for example, an instance in which a sensor results interface is rendered or brought into the foreground (e.g., in certain embodiments, to view any of the GUI described herein). In some embodiments, the update interval as described above, data on sensor results GUI 245 is automatically updated or refreshed according to an update interval (e.g., every second, every minute, every 5 minutes, etc.). As such, a “view” can comprise one instance per update interval in which a sensor results interface is rendered or brought into the foreground. For example, if the update interval is every minute, rendering or bringing into the foreground the sensor results GUI 245 several times in that minute would only comprise one “view.” Similarly, if the sensor results GUI 245 is rendered or brought into the foreground for 20 continuous minutes, data on the senor results GUI 245 would be updated 20 times (i.e., once every minute). However, this would only constitute 20 “views” (i.e., one “view” per update interval). Similarly, if the update interval is every five minutes, rendering or bringing into the foreground the sensor results GUI 245 several times in those five minutes would only comprise one “view.” If the sensor results interface is rendered or brought into the foreground for 20 continuous minutes, this would constitute 4 “views” (i.e., one “view” each for each of the four five-minute intervals). According to other embodiments, a “view” can be defined as an instance when a user views a sensor results interface with a valid sensor reading for the first time in a sensor lifecount. According to disclosed embodiments, user can receive a notification, as described below, indicating when an instance of rendering or brining into the foreground the sensor results GUI is not counted as a “view.” For example, the user can receive a visual notification indicating such as “Results have not updated,” or “View does not count,” or “Please check glucose level again.” In some embodiments, the user can receive a check-in for each instance which counts as a “view,” as described in greater detail below.

According to disclosed embodiments, the one or more processors can be configured to record no more than one instance of user operation of the reader device during a defined time period. For example, and not limitation, a defined time period can include an hour. A person of ordinary skill in the art would understand defined time period to include any appropriate period of time, such as, one hour, two hours, three hours, 30 minutes, 15 minutes, etc.

According to some embodiments, a “view” can comprise, for example, a visual notification (e.g., prompt, alert, alarm, pop-up window, banner notification, etc.). In some embodiments, the visual notification can include an alarm condition, an analyte level measurement associated with the alarm condition, and a trend indicator associated with the alarm condition. For example, Analyte Level/Trend Alert GUIs, such as those embodiments depicted in FIGS. 31A to 310 can constitute a “view.”

In some embodiments, a sensor user interface can include a visual display of a “scan” metric indicative of another measure of user engagement or interaction with the analyte monitoring system. A “scan” can comprise, for example, an instance in which a user uses a reader device (e.g., smart phone, dedicated reader, etc.) to scan a sensor control device, such as, for example, in a Flash Analyte Monitoring system. As described above in connection with “views”, a “scan” can comprise one instance per update interval in a user uses a reader device to scan a sensor control device.

FIGS. 32A and 32B depict example embodiments of sensor usage interfaces 500 and 510, respectively. In accordance with the disclosed subject matter, sensor usage interfaces 500 and 510 can be rendered and displayed, for example, by a mobile app or software residing in non-transitory memory of reader device 120, such as those described with respect to FIGS. 1 and 2A. In some embodiments, for each instance of a “views” or “scans,” the software can record the date and time of the user's interaction with the system. In some embodiments, for each instance of a “view” or “scan,” the software can record the current glucose value. Referring to FIG. 32A, sensor user interface 500 can comprise: a predetermined time period interval 508 indicative of a time period (e.g., a date range) during which view metrics are measured, a Total Views metric 502, which is indicative of a total number of views over the predetermined time period 508; a Views Per Day metric 504, which is indicative of an average number of views per day over the predetermined time period 508; and a Percentage Time Sensor Active metric 506, which is indicative of the percentage of predetermined time period 508 that reader device 120 is in communication with sensor control device 102, such as those described with respect to FIGS. 1, 2B, and 2C. Referring to FIG. 32B, sensor user interface 510 can comprise a Views per Day metric 504 and a Percentage Time Sensor Active metric 508, each of which is measured for predetermined time period 508.

According to another aspect of the embodiments, although predetermined time period 508 is shown as one week, those of skill in the art will recognize that other predetermined time periods (e.g., 3 days, 14 days, 30 days) can be utilized. In addition, predetermined time period 508 can be a discrete period of time—with a start date and an end date—as shown in sensor usage interface 500 of FIG. 32A, or can be a time period relative to a current day or time (e.g., “Last 7 Days,” “Last 14 Days,” etc.), as shown in sensor usage interface 510 of FIG. 32B.

FIG. 32C depicts an example embodiment of sensor usage interface 525, as part of analyte monitoring system report GUI 515. In accordance with the disclosed subject matter, GUI 515 is a snapshot report covering a predetermined time period 516 (e.g., 14 days), and comprising a plurality of report portions on a single report GUI, including: a sensor usage interface portion 525, a glucose trend interface 517, which can include an glucose trend graph, a low glucose events graph, and other related glucose metrics (e.g., Glucose Management Indicator); a health information interface 518, which can include information logged by the user about the user's average daily carbohydrate intake and medication dosages (e.g., insulin dosages); and a comments interface 519, which can include additional information about the user's analyte and medication patterns presented in a narrative format. According to embodiments, health information interface 518 can include a graphical representation of average glucose level over a day relative to the foregoing target glucose range (shown with horizontal lines at 80 and 180 mg/dL). According to embodiments, health information interface 518 can include a personalized-target glucose range report, such as those disclosed in International Publication No. WO2020/086934 to Xu, which is incorporated by reference in its entirety herein. According to embodiments, the personalized-target glucose range report can include a graphical representation of glucose level over a day relative to the foregoing personalized-target glucose range. According to another aspect of the embodiments, sensor usage interface 525 can comprise a Percentage Time Sensor Active metric 526, an Average Scans/Views metric 527 (e.g., indicative of an average sum of a number of scans and a number of views), and a Percentage Time Sensor Active graph 528. As can be seen in FIG. 32C, an axis of the Percentage Time Sensor Active graph can be aligned with a corresponding axis of one or more other graphs (e.g., average glucose trend graph, low glucose events graph), such that the user can visually correlate data between multiple graphs from two or more portions of the report GUI by the common units (e.g., time of day) from the aligned axes.

FIG. 32D depicts an example embodiment of another analyte monitoring system report GUI 530 including sensor usage information. In accordance with the disclosed subject matter, GUI 530 is a monthly summary report including a first portion comprising a legend 531, wherein legend 531 includes a plurality of graphical icons each of which is adjacent to a descriptive text. As shown in FIG. 32D, legend 531 includes an icon and descriptive text for “Average Glucose,” an icon and descriptive text for “Scans/Views,” and an icon and descriptive text for “Low Glucose Events.” GUI 530 also includes a second portion comprising a calendar interface 532. For example, as shown in FIG. 32D, GUI 530 comprises a monthly calendar interface, wherein each day of the month can include one or more of an average glucose metric, low glucose event icons, and a sensor usage metric 532. In some embodiments, such as the one shown in FIG. 32D, the sensor usage metric (“scans/views”) is indicative of a total sum of a number of scans and a number of views for each day. According to embodiments, an average glucose metric can include a personalized average glucose metric.

FIG. 32E depicts an example embodiment of another analyte monitoring system report GUI 540 including sensor usage information. In accordance with the disclosed subject matter, GUI 540 is a weekly summary report including a plurality of report portions, wherein each report portion is representative of a different day of the week, and wherein each report portion comprises a glucose trend graph 541, which can include the user's measured glucose levels over a twenty-four hour period, and a health information interface 543, which can include information about the user's average daily glucose, carbohydrate intake, and/or insulin dosages. In some embodiments, glucose trend graph 541 can include sensor usage markers 542 to indicate that a scan, a view, or both had occurred at a particular time during the twenty-four hour period. According to embodiments, glucose trend graph 541 can include the user's personalized glucose levels over a twenty-four hour period. According to embodiments, glucose trend graph 541 can include a personalized-target average glucose report, which can include a graphical representation of a subject's average glucose (for example, not limitation, shown by a solid line) over time and the personalized-target average glucose. According to embodiments, health information interface 543 can include information about the user's personalized average daily glucose.

FIG. 32F depicts an example embodiment of another analyte monitoring system report GUI 550 including sensor usage information. In accordance with the disclosed subject matter, GUI 550 is a daily log report comprising a glucose trend graph 551, which can include the user's glucose levels over a twenty-four hour period. According to embodiments, glucose trend graph 541 can include the user's personalized glucose levels over a twenty-four hour period. In some embodiments, glucose trend graph 551 can include sensor usage markers 552 to indicate that a scan, a view, or both had occurred at a particular time during the twenty-four hour period. Glucose trend graph 551 can also include logged event markers, such as logged carbohydrate intake markers 553 and logged insulin dosage markers 554, as well as glucose event markers, such as low glucose event markers 555.

According to embodiments, FIGS. 32A-F could additionally include laboratory measured HbA1c (“Lab A1c”).

### Example Embodiments of Digital Interfaces for Analyte Monitoring Systems

Described herein are example embodiments of digital interfaces for analyte monitoring systems. In accordance with the disclosed subject matter, a digital interface can comprise a series of instructions, routines, subroutines, and/or algorithms, such as software and/or firmware stored in a non-transitory memory, executed by one or more processors of one or more devices in an analyte monitoring system, wherein the instructions, routines, subroutines, or algorithms are configured to enable certain functions and inter-device communications. As an initial matter, it will be understood by those of skill in the art that the digital interfaces described herein can comprise instructions stored in a non-transitory memory of a sensor control device 102, reader device 120, local computer system 170, trusted computer system 180, and/or any other device or system that is part of, or in communication with, analyte monitoring system 100, as described with respect to FIGS. 1, 2A, and 2B. These instructions, when executed by one or more processors of the sensor control device 102, reader device 120, local computer system 170, trusted computer system 180, or other device or system of analyte monitoring system 100, cause the one or more processors to perform the method steps described herein. Those of skill in the art will further recognize that the digital interfaces described herein can be stored as instructions in the memory of a single centralized device or, in the alternative, can be distributed across multiple discrete devices in geographically dispersed locations.

### Example Embodiments of Reports Comprising a Plurality of Interfaces

Example embodiments of reports comprising a plurality of interfaces will now be described. According to embodiments, a report of the combined data from analyte monitoring systems and laboratory results from EMRs can be received by HCPs, caregivers, and/or analyte monitoring system users. In accordance with the disclosed subject matter, a report including a plurality of the interfaces disclosed herein may be presented to a user. In accordance with the disclosed subject matter, the interfaces can include any combination of measured interfaces based on current or measured analyte values, physiological parameter interfaces based on the physiological parameters disclosed herein, and personalized interfaces based on personalized glucose metrics disclosed herein.

In view of the above and in accordance with the disclosed subject matter, a glucose monitoring system is provided comprising a sensor control device, comprising an analyte sensor coupled with sensor electronics and configured to transmit data indicative of an analyte level of a subject, and a reader device. The reader device of the disclosed subject matter comprises a wireless communication circuitry configured to receive the data indicative of the analyte level and a glycated hemoglobin level for the subject, a non-transitory memory, and at least one processor. The processor is communicatively coupled to the non-transitory memory and the analyte sensor and configured to calculate a plurality of personalized glucose metrics for the subject using at least one physiological parameter and at least one of the received data indicative of the analyte level or the received glycated hemoglobin level, and display, on a display of the reader device, a report comprising a plurality of interfaces including at least two or more of the received data indicative of the analyte level, the received glycated hemoglobin level, or the calculated plurality of personalized glucose metrics, wherein the plurality of interfaces comprising the report are based on a user type. According to embodiments, the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant. For example, not limitation, in further embodiments, the plurality of interfaces includes the at least one physiological parameter for the subject.

According to embodiments, contents of a report may vary based on different user types (for example, not limitation, subjects, health care providers, caretakers, etc.). As embodied herein, the plurality of interfaces comprising the report are predetermined based on the user type or can be selected by the user. According to embodiment, the user type includes a health care professional. For example, without limitation, in a further embodiment, the plurality of interfaces includes a glucose monitoring data interface, a glycated hemoglobin interface, a personalized A1c interface, a personalized glucose interface, a personalized average glucose, and a personalized time in range interface. According to embodiment, the user type includes the subject. For example, without limitation, in a further embodiment, the plurality of interfaces a glucose monitoring data interface, a glycated hemoglobin interface, a mean glucose interface, and a time in range interface.

According to embodiments, subjects using the analyte monitoring systems can only view graphical interfaces displaying measured analyte measurements, or personalized analyte measurements, but not both. For example, it can be beneficial to minimize confusion by showing graphical interfaces with slightly different data (such as between measured and personalized). As embodied herein, the selection of which interfaces can be included in a report is dependent on whether the personalized glucose metrics have been approved or designated for research purposes or clinical purposes by the appropriate regulatory authority.

According to embodiments, personalized glucose metrics can include one or more of a personalized A1c or adjusted A1c, glucose-determined A1c or calculated A1c, personalized glucose, personalized average glucose, and personalized time in rage. According to embodiments, at least one processor is configured to calculate a plurality of personalized glucose targets corresponding to the calculated plurality of personalized glucose metrics. According to embodiments, the plurality of interfaces further includes the plurality of personalized glucose targets. According to embodiments, personalized glucose targets can include one or more of personalized glucose target range and personalized target average glucose. According to embodiments, personalized glucose target range can include a personalized lower glucose limit and/or a personalized upper glucose limit.

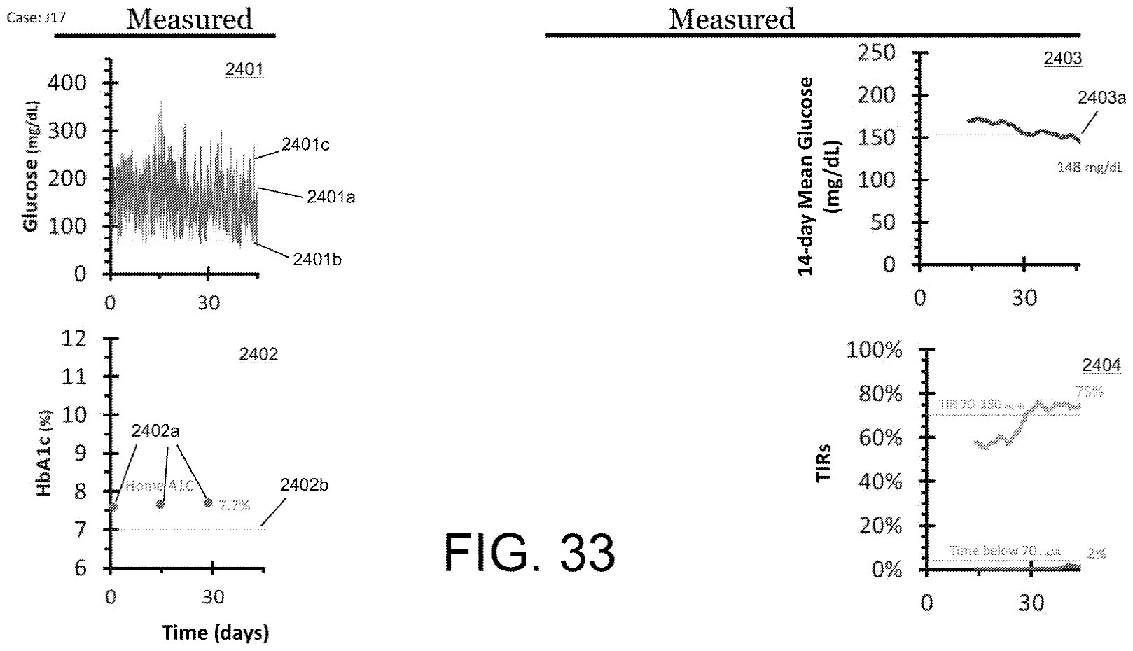

FIG. 33 shows an exemplary report 1400 including four different measured interfaces associated with exemplary subject J17: a glucose monitoring data interface 2401 which includes a graphical representation of measured glucose measurements from an analyte monitoring device over a predetermined period of time, HbA1c interface 2402 including a graphical representation of HbA1c measurements (shown as dots 1402a) over a predetermined period of time and a graphical representation of calculated A1c (“cA1c”) or glucose derived A1c, a mean glucose interface 1403 including a graphical representation of measured 14-day mean glucose (148 mg/dL) over a predetermined period of time, and time-in-range interface 1404 including a graphical representation of measured time in range metrics (75% over 180 mg/dL and 2% below 70 mg/dL, as shown) over a predetermined period of time. As embodied herein, HbA1c measurements can include laboratory A1c measurements. In further embodiment, for example, not limitation, the reader device wirelessly receives the glycated hemoglobin level for the subject from an electronic medical records system, cloud-based database, from the subject from a QR code, from the subject using a home test kit which can optionally be mailed to a laboratory for analysis. As embodied herein, FIG. 33 can include any of the interfaces disclosed herein.

As embodied herein, as shown in FIG. 33, glucose monitoring data interface 2401 can including a graphical representation (shown as dashed line) of target glucose range 2401b,c in the foreground. Target glucose range 2401b,c can include personalized target glucose range, as described herein. As embodied herein, as shown in FIG. 33, HbA1c interface 2402 can include a graphical representation (shown as solid line) of target HbA1c 2402b (for example, not limitation, 6.5%). As embodied herein, as shown in FIG. 33, mean glucose interface 1403 can include a graphical representation (shown as solid line) of target average glucose 1403a.

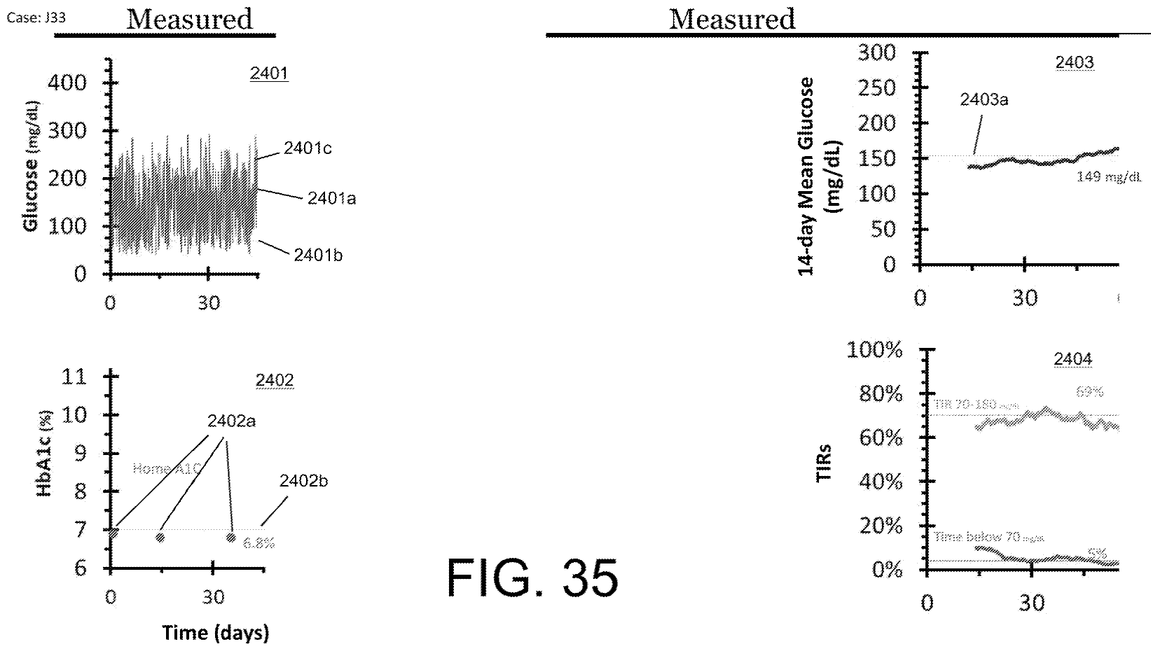

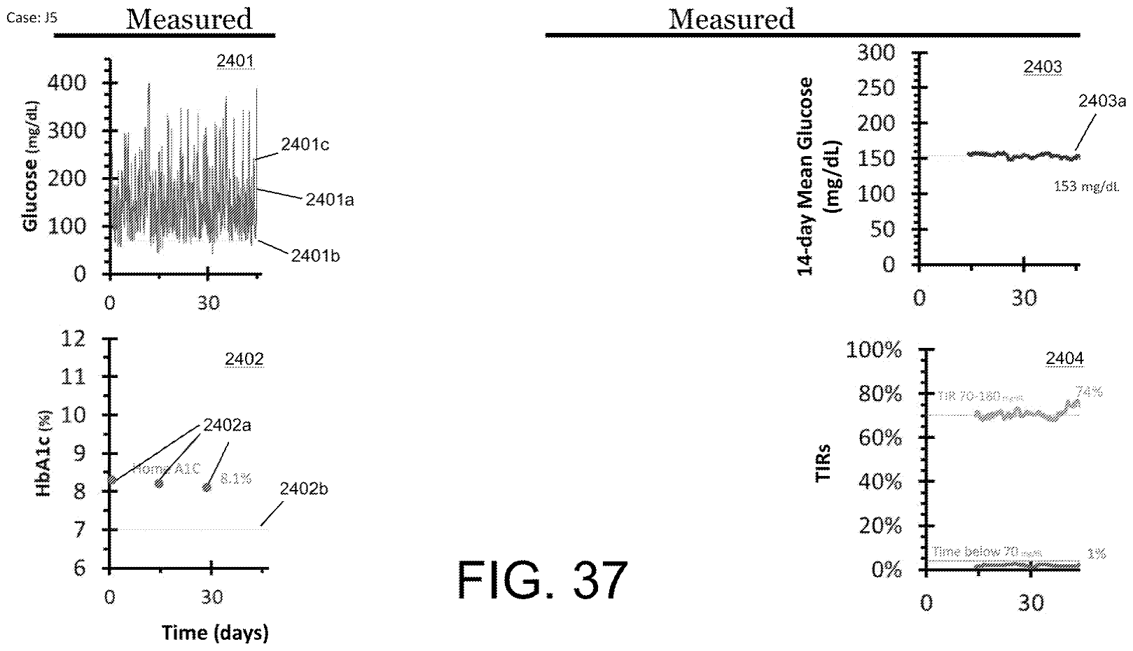

As embodied herein, as can be seen in FIG. 33, the predetermined time period can be 45 days. As embodied herein, the predetermined time period can be in five-minute increments, with a total of twelve hours of data. Those of skill in the art will appreciate, however, that other time increments (e.g., 30 days) and durations of analyte data can be utilized and are fully within the scope of this disclosure. FIGS. 35 and 37 similarly provide interfaces for exemplary subjects J33 and J5, respectively.

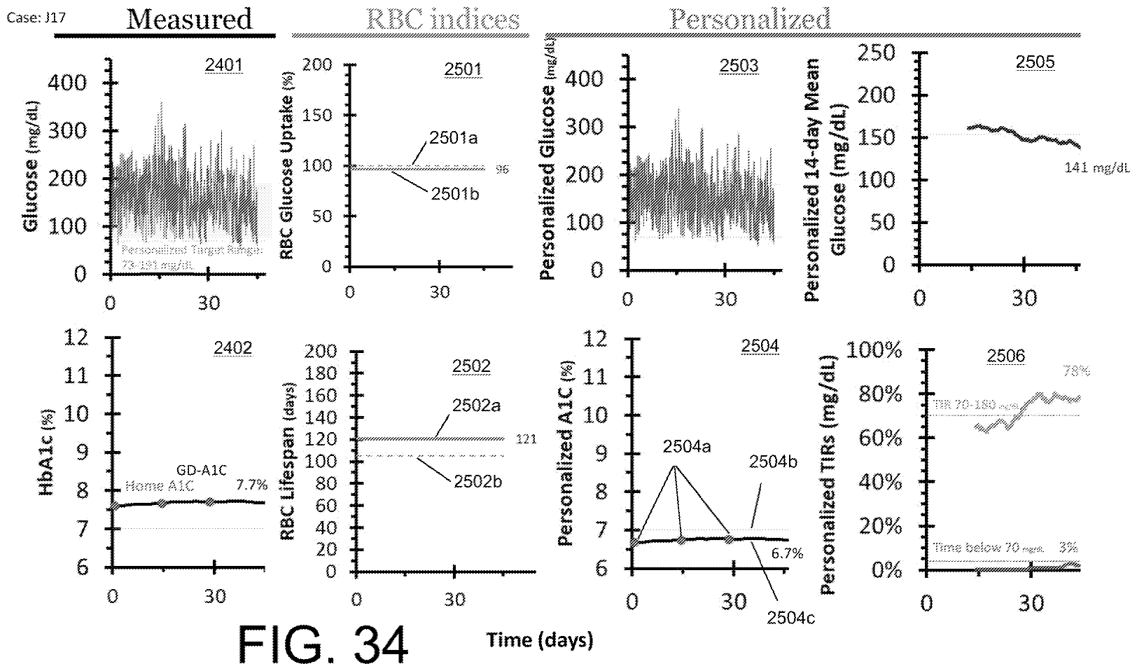

FIG. 34 shows an exemplary report 1500 including eight different interfaces associated with exemplary subject J17. As shown in FIG. 34, report 1500 can include measured interfaces, physiological parameter interfaces, and personalized interfaces. As embodied herein, FIG. 34 can include any of the interfaces disclosed herein.

According to embodiment disclosed herein, measured interfaces can include, for example, not limitation, a glucose monitoring data interface 2401 and HbA1c interface 2402, as shown in FIG. 34. As embodied herein, HbA1c interface 2402 can include a calculated HbA1c (cA1c or GD-A1c) curve fitted through the HbA1c measurements, as described herein and in WO2021/108419 and WO2020/086934 to Xu, which are incorporated by reference in its entirety herein.

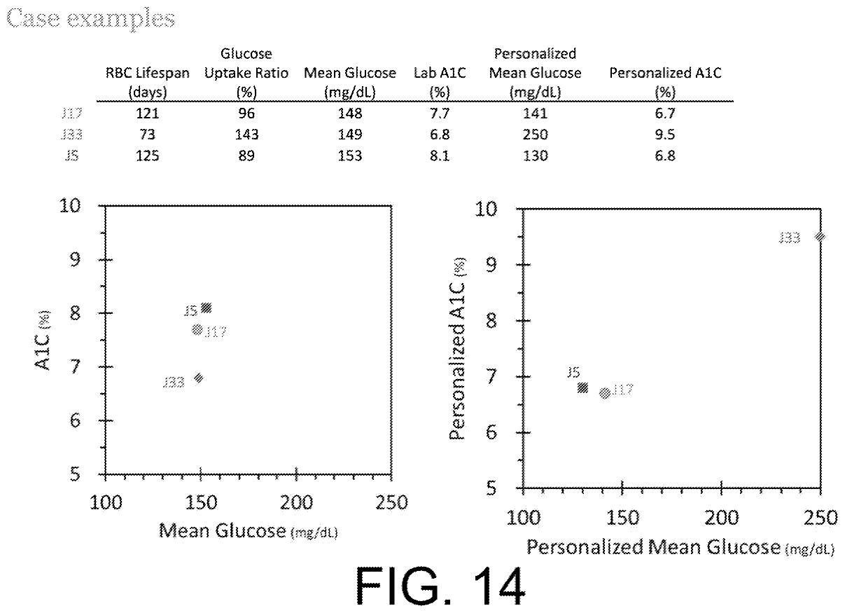

According to embodiment disclosed herein, physiological parameter interfaces can include for example, not limitation, red blood cell glucose uptake interface 2501 and red blood cell lifespan interface 2502, as shown in FIG. 34. As embodied herein, red blood cell glucose uptake interface 2501 can include a graphical representation of the subject's red blood cell glucose uptake (solid line) 2501a and a reference red blood cell glucose uptake (dashed line) 2501b over a predetermined period of time. As embodied herein, red blood cell lifespan interface 2502 can include a graphical representation of the subject's red blood cell lifespan (solid line) 2502a and a reference red blood cell lifespan (dashed line) 2502b over a predetermined period of time. As can be seen in FIG. 14 and illustrated in FIG. 34, subject J17's red blood cell glucose uptake is 96% and red blood cell lifespan is 121 days. The subject's red blood cell glucose uptake and red blood cell lifespan can be calculated using the models, described herein and in WO2021/108419 and WO2020/086934 to Xu, which are incorporated by reference in its entirety herein. As embodied herein, physiological parameter interfaces can include any other physiological parameters as described herein and in WO2021/108419 and WO2020/086934 to Xu, which are incorporated by reference in its entirety herein.

According to embodiment disclosed herein, personalized interfaces can include for example, not limitation, personalized glucose interface 2503, personalized A1c interface 2504, personalized 14-day mean glucose interface 2505, and personalized time in ranges interface 2506, as shown in FIG. 34. As embodied herein, FIG. 34 can include any of the personalized interfaces disclosed herein. Personalized glucose interface 2503 can include a graphical representation of the subject's glucose monitoring data interface personalized using the models as described herein and in WO2021/108419 and WO2020/086934 to Xu, which are incorporated by reference in its entirety herein. As embodied herein, as shown in FIG. 34, personalized glucose interface 2503 can including target glucose range 2401b,c in the foreground. Target glucose range 2401b,c can include personalized target glucose range, as described herein.

According to embodiment disclosed herein, personalized A1c interface 2504 can include a graphical representation of the subject's adjusted or personalized A1c (shown as a dots 2504a) and adjusted cHbA1c (shown as curve fit 2504c), calculated using the models as described herein and in WO2021/108419 and WO2020/086934 to Xu, which are incorporated by reference in its entirety herein. As embodied herein, personalized A1c interface 2504 can include a graphical representation (shown as solid line) of target HbA1c 2504b (for example, not limitation, 6.5%).

According to embodiment disclosed herein, personalized 14-day mean glucose interface 2505 can include a mean glucose interface 1403 including a graphical representation of personalized 14-day mean glucose (141 mg/dL as shown) over a predetermined period of time. As embodied herein, as shown in FIG. 34, personalized mean glucose interface 2503 can include a graphical representation (shown as solid line) of target average glucose 1403a.

According to embodiment disclosed herein, personalized time in ranges interface 2506, can include a graphical representation of personalized time in range metrics (78% over 180 mg/dL and 3% below 70 mg/dL, as shown) over a predetermined period of time.

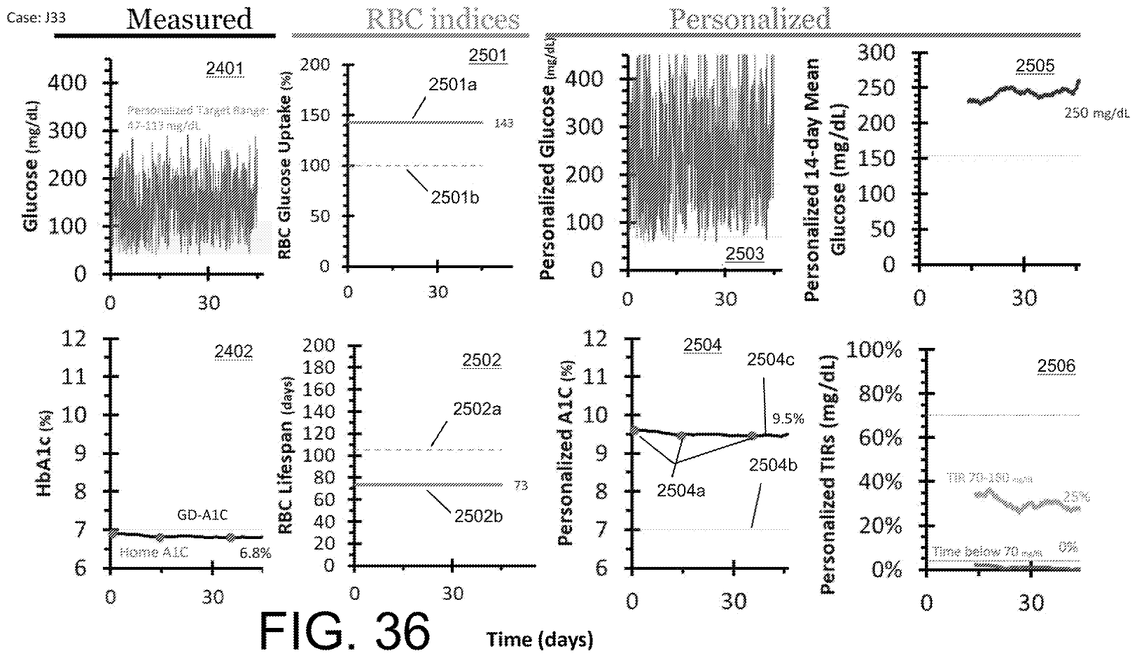

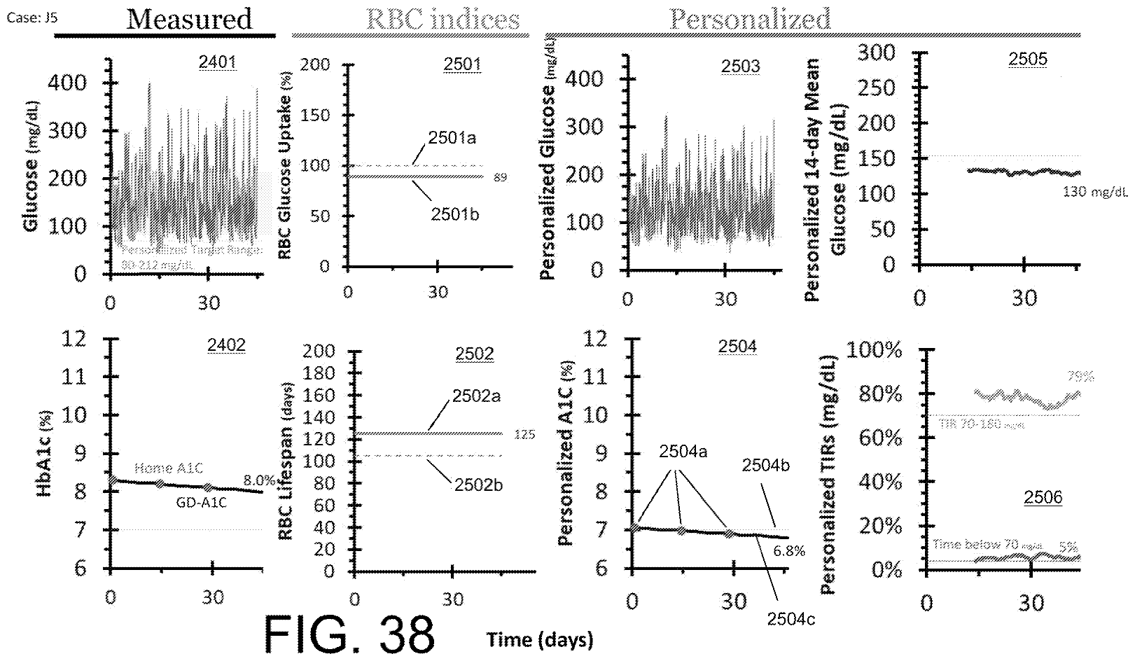

As embodied herein, as can be seen in FIG. 34, the predetermined time period can be 45 days. As embodied herein, the predetermined time period can be in five-minute increments, with a total of twelve hours of data. Those of skill in the art will appreciate, however, that other time increments, and durations of analyte data can be utilized and are fully within the scope of this disclosure. FIGS. 36 and 38 similarly provide graphical illustration of four different glucose metrics for J33 and J5, respectively.

According to embodiments disclosed herein, reports 1400 or 1500 can include a variety of measured interfaces, physiological parameter interfaces, or personalized interfaces based on user type. For example, health care providers (HCPs) and caretakers may benefit from seeing a comparison of measured interfaces and personalized interfaces, for example, to assess how much the two differ and to assess diagnosis and treatment options accordingly. As such, in an embodiment, contents of a report for an HCP can include a predetermined set of measured interfaces, physiological parameter interfaces, and personalized interfaces, for example, not limitation, as shown in report 1500. According to embodiments, HCPs can have the greatest access to information, including measured analyte measurements, personalized analyte measurement, and physiological parameters (for example, not limitation, RBC glucose uptake and RBC lifespan as shown in FIGS. 34, 36, 38, or as a second report as discussed above) as determined using models described herein. As embodied herein, in an embodiment, contents of a report for the subject can include a predetermined set of measured interfaces, physiological parameter interfaces, and/or personalized interfaces. For example, not limitation, a report generated for a user can include measured interfaces, as shown in report 1400. As embodied herein, a user type can include, for example, not limitation, the subject, a health care provider, a caretaker, an insurance provider, etc.

As embodied herein, a user (e.g., the subject, a HCP, a caretaker, an insurance provider, etc.) may select which interfaces comprise the report. For example, not limitation, the user may choose any combination of measured interfaces, personalized interfaces, and physiological parameter interface disclosed herein.

According to an embodiment, a user can select whether to view a sensor result interface as disclosed herein displaying measured analyte measurement (for example, not limitation, such as those shown in FIGS. 33, 35, and 27) over a predetermined period of time, or personalized measurements (for example, not limitation, such as those shown in FIGS. 34, 36, and 38) over the same predetermined period of time, or both. As embodied herein, the user can toggle or switch between viewing a sensor result interface with measured analyte measurements over a predetermined period of time and viewing the same sensor interface with personalized analyte measurements over the same predetermined period of time. For example, not limitation, a user can switch between a mean glucose interface 1403 including a graphical representation of average glucose level over 45 a day (for example, not limitation, such as that shown in FIG. 33) and a personalized mean glucose interface 2505 including a graphical representation of personalized average glucose level over the same predetermined period of time (for example, not limitation, such as that shown in FIG. 33). According to embodiments, a user can similarly switch between any of the other measured interfaces shown in FIGS. 33, 35, 37 and personalized interfaces shown in FIG. 34, 36, 38 (for example, without limitation, A1c interface, glucose interface, 14-day mean glucose interface, and time in range interface, etc.). Those of skill in the art will appreciate, however, that other time increments and durations of analyte data can be utilized and are fully within the scope of this disclosure. According to embodiments, the sensor results interfaces, analyte level and trend alert interfaces, time in range interfaces, and/or sensor usage interfaces as described herein can similarly be selected by a user to display measured analyte measurements over a predetermined period of time, and/or personalized analyte measurements over a predetermined period of time.

According to embodiments, with SMART-FHIR based applications, the provider could also access the report or a dashboard with the report directly through the EMR system. According to embodiments, HCPs may receive a different report or “dashboard” from caregivers and/or users. For example, not limitation, HCPs may receive a detailed report showing the combined data. Examples of a detailed report can include graphical representation of analyte measurements over a period of time, overlayed with laboratory measurements (e.g., in certain embodiments, HbA1c), graphical representation with various icons representing different laboratory results. According to embodiments, period of time can be selected by HCPs and can include, without limitation, 1 day, 5 days, 7 days, 14 days, 2 weeks, 1 month, 3 months, or any other period of time that may be clinically relevant. The HCP can use the combined data to provide patients with targets, for example, without limitation, “HbA1c level of 6.4% on your next visit.”

According to embodiments, the user may receive insights or encouragement based on the combined data. For example, not limitation, a user may receive a notification. Notifications can include, without limitation, “Based on your laboratory results and analyte measurements, we predict your HbA1c to be X.” According to embodiments, the notification may additionally state, “If you exercise/change diet/etc., your HbA1c level may lower to Y.”

According to embodiments, the combined data can be used in conjunction with any of the graphical user interfaces described above. According to embodiments, the user can personalize any of the graphical interfaces described above to alternatively or additionally display data received from EMR 5006.

Embodiments disclosed herein include:

K. A glucose monitoring system includes a sensor control device comprising an analyte sensor coupled with sensor electronics, the sensor control device configured to transmit data indicative of an analyte level of a subject, and a reader device comprising: a wireless communication circuitry configured to receive the data indicative of the analyte level and a glycated hemoglobin level for the subject, a non-transitory memory, at least one processor communicatively coupled to the non-transitory memory and the analyte sensor and configured to: calculate a plurality of personalized glucose metrics for the subject using at least one physiological parameter and at least one of the received data indicative of the analyte level or the received glycated hemoglobin level, and display, on a display of the reader device, a report comprising a plurality of interfaces including at least two or more of the received data indicative of the analyte level, the received glycated hemoglobin level, or the calculated plurality of personalized glucose metrics, wherein the plurality of interfaces comprising the report are based on a user type.

Embodiment K may have one or more of the following additional elements in any combination: Element 1: wherein the plurality of personalized glucose metrics includes one or more of an adjusted A1c, a calculated A1c, an adjusted calculated A1c, a personalized glucose, a personalized average glucose, or a personalized time in range. Element 2: wherein the at least one processor is further configured to calculate a plurality of personalized glucose targets corresponding to the calculated plurality of personalized glucose metrics. Element 3: wherein the plurality of interfaces further includes the plurality of personalized glucose targets. Element 4: wherein the plurality of personalized glucose targets includes one or more of a target glucose range or a target average glucose. Element 5: wherein the personalized target glucose range includes a personalized lower glucose limit. Element 6: wherein the personalized target glucose range includes a personalized upper glucose limit. Element 7: wherein the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant. Element 8: wherein the plurality of interfaces further includes the at least one physiological parameter for the subject. Element 9: wherein the user type includes a health care professional. Element 10: wherein the plurality of interfaces includes a glucose monitoring data interface, a glycated hemoglobin interface, a personalized a1c interface, a personalized glucose interface, a personalized average glucose, and a personalized time in range interface. Element 11: wherein the user type includes the subject. Element 12: wherein the plurality of interfaces includes a glucose monitoring data interface, a glycated hemoglobin interface, a mean glucose interface, and a time in range interface. Element 13: wherein the plurality of interfaces comprising the report are predetermined based on the user type. Element 14: wherein the plurality of interfaces comprising the report can be selected by the user. Element 15: wherein the at least one processor is further configured to output a notification if at least one of the plurality of personalized glucose metrics is at or above the corresponding plurality of personalized glucose target. Element 16: wherein the notification comprises a visual notification. Element 17: wherein the notification comprises an audio notification. Element 18: wherein the notification is an alarm. Element 19: wherein the notification is a prompt. Element 20: wherein the reader device wirelessly receives the glycated hemoglobin level for the subject from an electronic medical records system. Element 21: wherein the reader device wirelessly receives the glycated hemoglobin level for the subject from a cloud-based database. Element 22: wherein the reader device wirelessly receives the glycated hemoglobin level for the subject from a QR code. Element 23: the reader device wirelessly receives the glycated hemoglobin level for the subject from a home test kit.

While the disclosed subject matter is described herein in terms of certain illustrations and examples, those skilled in the art will recognize that various modifications and improvements may be made to the disclosed subject matter without departing from the scope thereof. Moreover, although individual features of one embodiment of the disclosed subject matter may be discussed herein or shown in the drawings of one embodiment and not in other embodiments, it should be apparent that individual features of one embodiment may be combined with one or more features of another embodiment or features from a plurality of embodiments.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for the purposes of illustration and description. It is not intended to be exhaustive or to limit the disclosed subject matter to those embodiments disclosed.

The description herein merely illustrates the principles of the disclosed subject matter. Various modifications and alterations to the described embodiments will be apparent to those skilled in the art in view of the teachings herein. Accordingly, the disclosure herein is intended to be illustrative, but not limiting, of the scope of the disclosed subject matter.

What is claimed is:

1. A method comprising:
receiving, using one or more processors, a first record including a first data associated with a personal identification from a first database;
receiving, using the one or more processors, a second record including a second data associated with a user identification from a second database;
pairing, using the one or more processors, the first data and the second data based upon a shared data item contained in the first record and the second record; and
displaying, using one or more processors, a report based upon the first data and the second data.

2. The method of claim 1, wherein the first database is an electronic medical record system and the first data is laboratory measured HbA1c.

3. The method of claim 1, wherein the second database includes an analyte monitoring system data service and the second data includes glucose levels measured by an analyte monitoring system.

4. The method of claim 1, wherein the shared data item includes an email address.

5. The method of claim 1, further comprising generating, using the one or more processors, a notification based upon the first data paired with the second data.

6. The method of claim 1, further comprising calculating, using the one or more processors, a plurality of personalized glucose metrics using at least one physiological parameter and at least one of the first record or the second record,
wherein the first record is laboratory measured HbA1c and the second record is glucose level data indicative of an analyte level of a user,
wherein the report comprises a plurality of interfaces including two or more of: the first record, the second record, or the calculated plurality of personalized glucose metrics, and
wherein the plurality of interfaces comprising the report are based on a user type.

7. The method of claim 6, wherein the plurality of personalized glucose metrics includes one or more of an adjusted A1c, a calculated A1c, an adjusted calculated A1c, a personalized glucose, a personalized average glucose, or a personalized time in range.

8. The method of claim 7, further comprising calculating a plurality of personalized glucose targets corresponding to the calculated plurality of personalized glucose metrics.

9. The method of claim 8, wherein the plurality of interfaces further includes the plurality of personalized glucose targets.

10. The method of claim 8, wherein the plurality of personalized glucose targets includes one or more of a target glucose range or a target average glucose.

11. The method of claim 10, wherein the personalized target glucose range includes a personalized lower glucose limit.

12. The method of claim 10, wherein the personalized target glucose range includes a personalized upper glucose limit.

13. The method of claim 6, wherein the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant.

14. The method of claim 13, wherein the plurality of interfaces further includes the at least one physiological parameter for the user.

15. The method of claim 6, wherein the user type includes a health care professional.

16. The method of claim 15, wherein the plurality of interfaces includes a glucose monitoring data interface, a glycated hemoglobin interface, a personalized a1c interface, a personalized glucose interface, a personalized average glucose, and a personalized time in range interface.

17. The method of claim 6, wherein the user type includes the user.

18. The method of claim 17, wherein the plurality of interfaces includes a glucose monitoring data interface, a glycated hemoglobin interface, a mean glucose interface, and a time in range interface.

19. The method of claim 6, wherein the plurality of interfaces comprising the report are predetermined based on the user type.

20. The method of claim 6, wherein the plurality of interfaces comprising the report can be selected by the user.

21. The method of claim 9, further comprising outputting a notification if at least one of the plurality of personalized glucose metrics is at or above the corresponding plurality of personalized glucose target.

22. The method of claim 21, wherein the notification comprises a visual notification.

23. The method of claim 21, wherein the notification comprises an audio notification.

24. The method of claim 21, wherein the notification is an alarm.

25. The method of claim 21, wherein the notification is a prompt.

26. The method of claim 6, wherein the reader device wirelessly receives the glycated hemoglobin level for the user from an electronic medical records system.

27. The method of claim 6, wherein the reader device wirelessly receives the glycated hemoglobin level for the user from a cloud-based database.

28. The method of claim 6, wherein the reader device wirelessly receives the glycated hemoglobin level for the user from a QR code.

29. The method of claim 6, the reader device wirelessly receives the glycated hemoglobin level for the user from a home test kit.

30. A glucose monitoring system, comprising:
a sensor control device comprising an analyte sensor coupled with sensor electronics, the sensor control device configured to transmit data indicative of an analyte level of a user; and
a reader device comprising:
a wireless communication circuitry configured to receive the data indicative of the analyte level and a glycated hemoglobin level for the user;
a non-transitory memory;
at least one processor communicatively coupled to the non-transitory memory and the analyte sensor and configured to:
calculate a plurality of personalized glucose metrics for the user using at least one physiological parameter and at least one of the received data indicative of the analyte level or the received glycated hemoglobin level; and
display, on a display of the reader device, a report comprising a plurality of interfaces including at least two or more of the received data indicative of the analyte level, the received glycated hemoglobin level, or the calculated plurality of personalized glucose metrics,
wherein the plurality of interfaces comprising the report are based on a user type.

31. The system of claim 30, wherein the plurality of personalized glucose metrics includes one or more of an adjusted A1c, a calculated A1c, an adjusted calculated A1c, a personalized glucose, a personalized average glucose, or a personalized time in range.

32. The system of claim 31, wherein the at least one processor is further configured to calculate a plurality of personalized glucose targets corresponding to the calculated plurality of personalized glucose metrics.

33. The system of claim 32, wherein the plurality of interfaces further includes the plurality of personalized glucose targets.

34. The system of claim 32, wherein the plurality of personalized glucose targets includes one or more of a target glucose range or a target average glucose.

35. The system of claim 34, wherein the personalized target glucose range includes a personalized lower glucose limit.

36. The system of claim 34, wherein the personalized target glucose range includes a personalized upper glucose limit.

37. The system of claim 30, wherein the at least one physiological parameter is selected from the group consisting of: a red blood cell glucose uptake, a red blood cell lifespan, a red blood cell glycation rate constant, a red blood cell generation rate constant, a red blood cell elimination constant, and an apparent glycation constant.

38. The system of claim 37, wherein the plurality of interfaces further includes the at least one physiological parameter for the user.

39. The system of claim 30, wherein the user type includes a health care professional.

40. The system of claim 39, wherein the plurality of interfaces includes a glucose monitoring data interface, a glycated hemoglobin interface, a personalized a1c interface, a personalized glucose interface, a personalized average glucose, and a personalized time in range interface.

41. The system of claim 30, wherein the user type includes the user.

42. The system of claim 41, wherein the plurality of interfaces includes a glucose monitoring data interface, a glycated hemoglobin interface, a mean glucose interface, and a time in range interface.

43. The system of claim 30, wherein the plurality of interfaces comprising the report are predetermined based on the user type.

44. The system of claim 30, wherein the plurality of interfaces comprising the report can be selected by the user.

45. The system of claim 33, wherein the at least one processor is further configured to output a notification if at least one of the plurality of personalized glucose metrics is at or above the corresponding plurality of personalized glucose target.

46. The system of claim 38, wherein the notification comprises a visual notification.

47. The system of claim 38, wherein the notification comprises an audio notification.

48. The system of claim 38, wherein the notification is an alarm.

49. The system of claim 38, wherein the notification is a prompt.

50. The system of claim 30, wherein the reader device wirelessly receives the glycated hemoglobin level for the user from an electronic medical records system.

51. The system of claim 30, wherein the reader device wirelessly receives the glycated hemoglobin level for the user from a cloud-based database.

52. The system of claim 30, wherein the reader device wirelessly receives the glycated hemoglobin level for the user from a QR code.

53. The system of claim 30, the reader device wirelessly receives the glycated hemoglobin level for the user from a home test kit.
